(19) United States
(12) Patent Application Publication
(10) Pub. No.: US 2021/0355532 A1
(43) Pub. Date: Nov. 18, 2021

(54) METHODS AND REAGENTS FOR DETECTING AND ASSESSING GENOTOXICITY

(71) Applicant: TwinStrand Biosciences, Inc., Seattle, WA (US)

(72) Inventors: Jesse J. SALK, Seattle, WA (US); Charles Clinton VALENTINE, III, Seattle, WA (US)

(21) Appl. No.: 16/969,531

(22) PCT Filed: Feb. 13, 2019
(86) PCT No.: PCT/US19/17908

§ 371 Date: Dec. 7, 2020

Related U.S. Application Data
(60) Provisional application No. 62/630,228, filed on Feb. 13, 2018.
(60) Provisional application No. 62/737,097, filed on Sep. 26, 2018.

(51) Int. Cl. C12Q 1/6869; C12Q 1/6883; G16B 20/50; G16B 30/00

(52) U.S. Cl. CPC C12Q 1/6869; C12Q 1/6883; C12Q 2600/142; G16B 30/00; G16B 20/50

(57) ABSTRACT

Methods, systems, and kits with reagents for assessing genotoxicity, are disclosed herein. Genotoxicity and their mechanisms of action can be determined within a few days of a subjects exposure. Some embodiments of the technology are directed to utilizing Duplex Sequencing for assessing a genotoxic potential of a compound (e.g., a chemical compound) in an exposed subject. Other embodiments of the technology are directed to utilizing Duplex Sequencing for determining a mutation signature associated with a genotoxic agent; and/or a safe threshold level of genotoxin exposure. Additional embodiments of the technology are directed to identifying one or more genotoxic agents a subject may have been exposed to by comparing the subjects DNA mutation spectrum to the mutation spectra of known mutagenic compounds. Once a genotoxin exposure in a subject is identified, or confirmed, then a prophylactic, and/or inhibitory therapeutic course of treatment is provided.

### CROSS-REFERENCE TO RELATED APPLICATIONS

This is a U.S. National Stage application of Int. Appl. No. PCT/US2019/017908, filed Feb. 13, 2019, which claims priority to and the benefit of U.S. Provisional Patent Application No. 62/630,228, filed Feb. 13, 2018, and U.S. Provisional Patent Application No. 62/737,097, filed Sep. 26, 2018, the disclosures of which are hereby incorporated by reference in their entirety.

### BACKGROUND

Genotoxicity refers to the destructive property of agents or processes (i.e., genotoxins) that cause damage to genetic material (e.g., DNA, RNA). In germ cell lines, damage to nucleic acid material has the potential to result in a heritable germline mutation, while damage to nucleic acid material in somatic cells can result in a somatic mutation. In some instances, such somatic mutations may lead to malignancy or other diseases. It has been established that genotoxin exposure may directly or indirectly cause such nucleic acid damage, or in some instances may be responsible for both directly and indirectly triggering nucleic acid damage. For example, a genotoxic substance may directly interact with the genetic material to causes changes in the nucleotide sequence itself or the its structure or create chemical modifications (for example adducts or breaks) that when attempted to be copied, repaired or otherwise processed by cellular machinery, induce (or increase the probability of inducing) changes to the nucleotide sequence. The genotoxin may be a naturally occurring chemical or process (for example, coal, radium or UV light) or an artificially created chemical or process or therapy (for example industrial urethane, X-ray machines, many chemotherapy drugs, and some forms of gene therapy).

Other genotoxins may indirectly trigger the nucleic acid damage by activating cellular pathways that reduce the fidelity of DNA replication. For example this may be direct or indirect activation of cell-cycle machinery that bypasses normal checkpoints or by reducing normal repair of nucleic acids (such as direct or indirect dysregulation of any one of many nucleic acid repair pathways including mismatch repair (MMR), nucleotide excision repair (NER), base excision repair (BER), double-strand break repair (DSBR), transcription-coupled repair (TCR), non-homologous end joining (NHEJ), among others). Other genotoxins may indirectly act by promoting cellular environment that is, itself, genotoxic. One example of such an environment is “oxidative stress”, which can be created by increasing reactive oxygen species production in an organism (for example through stimulation of immune mediated inflammation) or cell that can cause damage to the genetic material by either modifying a sequence chemical composition itself or structurally altering nucleic acid strands. Yet another indirect form of genotoxins are agents or processes which suppress certain aspects of the immune system of an organism. Such reductions in immune surveillance can lead to genotoxicity in an organism by allowing the proliferation of microorganisms that may be genotoxic through any one of several mechanisms (for example, by causing inflammation or promoting cell-cycle progression in certain tissues). Furthermore, such agents or processes can contribute to the genotoxic load of an organism via reduction of the normal capacity to purge cells bearing genetic abnormalities that would otherwise be cleared and be carcinogenic via this mechanism. The mechanisms of many genotoxins remain to be discovered.

Genotoxins can originate from a variety of external and internal sources. For example, external (i.e., exogenous) sources, can include chemicals or a mixture of chemicals (e.g. pharmaceuticals, industrial/manufacturing byproducts, chemical waste, cosmetics, household cleaners, plasticizers, tobacco smoke, solvents, etc.); heavy metals, airborne particles, contaminants, food products, radiation (e.g., photons, such as gamma radiation, X-radiation, particle radiation or a mix thereof), physical forces (e.g. a magnetic field, gravitational field, acceleration forces, etc.) from the natural environment or from a device; another organism (e.g. viruses, parasites, bacteria, protozoa, fungi) or produced by another naturally-occurring organism (e.g., fungus, plant, animal, bacteria, bacteria, protozoa etc.). Certain crops themselves (for example tobacco) contain known genotoxins in their natural form. Staple food crops may become contaminated with genotoxins during growth (for example, contamination of irrigation water with industrial waste), harvest (for example inadvertent co-harvest of crops with aristocholia, which produce the mutagen aristolochic acid), storage (for example damp legume and grain silos leading to growth of aspergillus species that produce the mutagen aflatoxin), or during preparation (for example, smoking and some other preservation methods of meats, which create many forms of genotoxins or high temperature cooking of starches which may produce the mutagen acrylamide). Some examples of internal (i.e., endogenous) sources may include biochemical processes or the results of biochemical processes. For example, a chemical agent may be determined to be a genotoxin if the agent is a precursor to a mutagen that results from metabolic activation. Other examples might include stimulators of inflammatory pathways (e.g. stress, autoimmune disease), or inhibitors of apoptosis or immune surveillance. Regardless of the source, a number of factors play a role in determining whether an agent or process is potentially genotoxic, mutagenic or carcinogenic (i.e., cancer-causing).

In certain applications, the ability to detect and quantify mutagenic processes is important for assessing cancer risk and predicting the impact of carcinogenic exposure in humans. Likewise, assessing the potential for chemical compounds or other agents to cause nucleic acid mutations is an essential element of product safety testing before marketing (e.g., pharmaceuticals, cosmetics, food products, manufacturing byproducts and the like). Current methods of identifying genotoxins are laborious, costly, time delayed (e.g. years between exposure and symptoms), may not be representative of the true in-human effect (verses only certain model organisms) and in some cases, present with difficulty to pinpoint the exact causative agent. For example, on occasion a detection of an increased incidence of a population of subjects becoming ill (for example, cancer clusters) is necessary before a search for a genotoxin is initiated (e.g. pharmaceutical and food safety analysis, environmental contaminant or investigation of environmental dumping, etc.).

Conventional measures of somatic mutation in vivo are indirectly inferred from selection-based assays in bacteria, cell culture, or transgenic animals where the genome-wide effect is extrapolated from a small artificial reporter. Accordingly, currently used assays are imperfect surrogates for the true genotoxic potential of a compound in vivo, and they are labor intensive, while only providing a limited subset of information about a compound's mutagenic potential. It is likely that many compounds showing mutagenic potential in artificial bacterial systems (i.e., the Ames assay), do not accurately reflect a genuine risk in humans, and cause otherwise therapeutically promising compounds to be unnecessarily pulled from development or commercial use. Similarly, some compounds with carcinogenic potential do so through non-direct mutagenic mechanisms that are undetectable in bacteria. Such compounds could cause harm to subjects, as risk cannot be adequately recognized early.

In vivo mammalian reporter systems, such as transgenic rodent assays (e.g., the BigBlue® mouse and rat, and Muta™Mouse), offer a better approximation of human drug effect than bacteria. Although they are limited insofar as animals are not perfect representations of humans, mammalian transgenic assays remain valuable for early pre-clinical safety testing; however, these assays are complex and are still somewhat artificial. The BigBlue® assay, for example, relies on a reporter-based system whereby a subset of mutations that occur in a multi-copy lambda-phage transgene can be phenotypically identified after recovery of the reporter by a shuttle vector that is then transfected into bacteria. Not all mutations that occur in the 294 BP reporter gene can be detected, since many do not confer a phenotype. The transgene itself is highly condensed, methylated and does not represent the highly variable transcriptional and condensation state of the broader genome. Passing mutant molecules through viral and bacterial machinery has the potential to introduce artifactual mutations and the inherent bottle-necking that occurs at each step means that the allele fraction of mutations is non-quantitative. Furthermore, testing requires use of specific strains of a limited subset of species. And rodents themselves are not perfect representations of humans. For example, aflatoxin is highly mutagenic in humans, but is not meaningfully carcinogenic in mice after sexual maturity when certain metabolic enzymes become expressed, which facilitate its detoxification. Although transgenic rodents remain a current gold standard accepted by the U.S. Food and Drug Administration (FDA) and other regulatory agencies as a valid genotoxicity metric that can be used as a carcinogenicity surrogate in some testing situations, it is far from optimal as a broadly usable tool for assessing the potential for a compound to cause cancer in humans.

A fast, flexible, reliable method is needed that allows direct measurement of the genotoxic potential of factors/agents/environments a subject may be exposed to that cause nucleic acid mutations and damage contributing to certain health risks (i.e. cancer/malignancy/neoplasm, neurotoxicity, neurodegeneration, infertility, birth defects etc.) The method should be useable in any genomic locus of any tissue type and/or cell type in any type of organism, and without the need for any clonal selection (as required in the prior art gold-standard tests), and while providing information (inferred or directly) on the mechanism of action of how the carcinogenic factor causes mutations or other genotoxic damage in vivo leading to cancer development or other diseases or disorders in the subject/organism, or another organism that is modeled by the subject/organism.

If a sufficiently accurate, expedient tool with these features were available, it would have many applications, e.g.: in both pre-clinical and clinical drug safety testing; in preventing, diagnosing and treating genotoxin associated diseases and disorders; in detecting and identifying mutation causative factors/agents and their mechanisms of action; and other industry-wide implications (e.g. environmental pollution testing and determining threshold levels of toxicity onset, high-throughput consumer product safety testing, patient diagnosing and treatment if suspected of toxic exposure, national security risk assessment of intentional or unintentional release of genotoxins etc.).

### SUMMARY

The present technology is directed to methods, systems, and kits of reagents for assessing genotoxicity. In particular, some embodiments of the technology are directed to utilizing Duplex Sequencing for assessing a genotoxic potential of a compound (e.g., a chemical compound) and/or an environment agent (e.g. radiation) in an exposed subject. For example, various embodiments of the present technology include performing Duplex Sequencing methods that allow direct measurement of compound-induced mutations in any genomic context of any organism, and without the need for any clonal selection. Further examples of the present technology are directed to methods for detecting and assessing genomic in vivo mutagenesis using Duplex Sequencing and associated reagents. Various aspects of the present technology have many applications in both pre-clinical and clinical drug safety testing as well as other industry-wide implications.

In an embodiment, the present technology comprises a method for detecting and quantifying genomic mutations developed in vivo in a subject following the subject's exposure to a mutagen, comprising: (1) Duplex Sequencing one or more target double-stranded DNA molecules extracted from a subject exposed to a mutagen; (2) generating an error-corrected consensus sequence for the targeted double-stranded DNA molecules; and (3) identifying a mutation spectrum for the targeted double-stranded DNA molecules; (4) calculating a mutant frequency for the target double-stranded DNA molecules by calculating the number of unique mutations per duplex base-pair, of one or more types, sequenced.

In another embodiment, the present technology comprises a method for generating a mutagenic signature of a test compound, comprising: (1) Duplex Sequencing DNA fragments extracted from a living organism, e.g. a test animal, exposed to the test compound; and (2) generating a mutagenic signature of the test compound. And the method may further comprise calculating a mutant frequency for a plurality of the DNA fragments by calculating the number of unique mutations per duplex base-pair sequenced.

In another embodiment, the present technology comprises a method for assessing a genotoxic potential of a compound, comprising: (1) duplex sequencing targeted DNA fragments extracted from a test animal exposed to the compound to generate error-corrected consensus sequences of the targeted DNA fragments; (2) generating a mutagenic signature of the compound from the error-corrected consensus sequences; and (3) determining if exposure to the compound resulted in a mutagenic signature representative of a sufficiently genotoxic compound.

In another embodiment, the present technology comprises kits comprising reagents with instructions for conducting the methods disclosed herein for detecting and quantifying genotoxins. The kits may further comprise a computer program product installed on an electronic computing device (e.g. laptop/desktop computer, tablet, etc.) or accessible via a network (e.g. remote server with a database of subject records and detected genotoxins). The computer program product is embodied in a non-transitory computer readable medium that, when executed on a computer, performs steps of the methods using the kits disclosed herein for detecting and identifying genotoxins.

In another embodiment, the present technology comprises a networked computer system to identify or confirm a subject's exposure to at least one genotoxin, comprising: (1) a remote server; (2) a plurality of user electronic computing devices able to utilize the kits disclosed herein to extract, amplify, sequence a subject's sample; (3) a third party database with known genotoxin profiles (optional); and (4) a wired or wireless network for transmitting electronic communications between the electronic computing devices, database, and the remote server. The remote server further comprises: (a) a database storing user genotoxin record results, and records of genotoxin profiles (e.g. spectrum, frequencies, mechanism of actions, etc.); (b) one or more processors communicatively coupled to a memory; and one or more non-transitory computer-readable storage devices or medium comprising instructions for processor(s), wherein said processors are configured to execute said instructions to perform operations comprising the steps of: correcting errors in Duplex Sequencing fragments; and computing the mutation spectrum, mutant frequency, and triplet mutation spectrum of detected agents, from which the identity of at least one genotoxin can be determined.

The present technology further comprises, a non-transitory computer-readable storage media comprising instructions that, when executed by one or more processors, performs a method for determining if a subject is exposed to and/or the identity of at least one genotoxin, the method comprising the steps of correcting errors in Duplex Sequencing fragments; and computing the mutation spectrum, mutant frequency, and triplet spectrum of detected agents, from which the identity of at least one genotoxin is determined.

The present technology further comprises a computerized method for determining if a subject is exposed to and/or the identity of at least one genotoxin, the method comprising the steps of correcting errors in Duplex Sequencing fragments; and computing the mutation spectrum, mutant frequency, and triplet spectrum of detected agents, from which the identity of at least one genotoxin is determined.

In another embodiment, the present technology comprises a method, system, and kit for diagnosing and treating a subject exposed to a genotoxin. Diagnosing comprises detecting at least one genotoxin the subject has been exposed to and/or consumed; and treating comprises removing future exposure and/or consumption of the genotoxin(s), and/or administering treatment protocols (e.g. pharmaceuticals) to block and/or otherwise counteract the biological effect of the genotoxin(s).

In another embodiment, the present technology comprises a method, computerized system, and kit for both pre-clinical and clinical drug safety testing; for detecting and identifying carcinogens and their mechanisms of action; and for other industry-wide implications (e.g. toxic environmental pollutants, high-throughput consumer product and drug safety testing, etc.).

In another embodiment, the present technology comprises a method, system, and kit identifying novel genotoxins using error corrected Duplex Sequencing, and/or then determining a safety threshold amount (weight, volume, concentration, etc.) and/or a safety threshold mutant frequency of a genotoxin a subject may be exposed to before the subject is at risk for developing a genotoxin associated disease or disorder (e.g. used in setting Environmental Protection Agency standards; used in diagnosing and treating a subject exposed to the genotoxin, etc.).

In another embodiment, the present technology comprises a method, system, and kit for preventing a subject from developing a mutation associated disease or disorder by determining if the subject was exposed to a genotoxin at more than a safety threshold level (i.e. genotoxin amount and/or genotoxin mutant frequency and triplet signature); and if so, then providing prophylactic treatment to prevent, inhibit, or deter disease onset.

One aspect of the present technology comprises the ability to detect mutations causing a disease, but within a few days or a few weeks or a few months or a few years after exposure to a mutation causing genotoxin. Normally, full disease onset is not diagnosed for many years (e.g. 10-20 years for lung cancer development post exposure to asbestos). The methods and kits disclosed herein enable the detection of genomic mutations that cause disease onset immediately after exposure, versus waiting years for symptoms to appear.

Another aspect of the present technology comprises the ability to predict if a subject has an increased risk of developing a disease or disorder due to genotoxin caused mutations within about 2-5 days at a minimum to years later after a potential exposure to the genotoxin; and if so, to provide prophylactic treatment and periodic screening to detect the disease onset in the early stages.

Another aspect comprises a DNA library, and method of making, comprising a plurality of double-stranded, isolated genomic DNA fragments, wherein each fragment is ligated to one or more desired adapter molecules.

Another aspect comprises a high throughput method for rapidly screening a plurality of compounds to identify which compounds are genotoxic.

Another aspect comprises a high throughput method for rapidly screening a plurality of different tissues/cells types of the same subject to determine if the subject has been exposed to any genotoxin.

Another aspect comprises a high throughput method for rapidly screening a plurality of tissues and cells derived from different subjects to determine the percentage of the population exposed to any genotoxin.

Another aspect comprises directly or inferentially determining the “mechanism of action” of the genotoxin that causes exposure of it to result in a mutation associated with a specific disease or disorder.

Other embodiments, aspects and advantages of the present technology are described further in the following detailed description.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A illustrates a nucleic acid adapter molecule for use with some embodiments of the present technology and a double-stranded adapter-nucleic acid complex resulting from ligation of the adapter molecule to a double-stranded nucleic acid fragment in accordance with an embodiment of the present technology.

FIGS. 1B and 1C are conceptual illustrations of various Duplex Sequencing method steps in accordance with an embodiment of the present technology.

FIG. 2A is a conceptual illustration of various method schemes for using in vivo animal studies to predict human cancer risk of a test compound including conventional, long-term rodent carcinogenicity studies (left-hand scheme), a conventional transgenic rodent mutagenicity study with ex vivo selection (middle scheme), and mutagenesis assessment via a direct DNA sequencing scheme in accordance with aspects of the present technology (right-hand scheme).

FIGS. 2B and 2C are conceptual illustrations of method schemes for using Duplex Sequencing for assessing in vitro mutagenesis of a test compound in human cells grown in culture (2B) and for assessing in vivo mutagenesis of a test compound in a wild type mouse (2C) in accordance with aspects of the present technology.

FIGS. 3A-3D are box plot graphs showing mutant frequencies calculated for Duplex Sequencing (FIGS. 3A and 3B) and BigBlue® cII plaque assay (FIGS. 3C and 3D) in liver and bone marrow following mutagen treatment and in accordance with an embodiment of the present technology.

FIG. 3E is a plot illustrating the relative cII mutant fold increase in the BigBlue® cII plaque assay versus the Duplex Sequencing assay of FIGS. 3A-3D, and in accordance with an embodiment of the present technology.

FIG. 3F shows the proportion of single nucleotide variants (SNV) within the cII gene for individually picked mutant plaques produced from BigBlue® mouse tissue and Duplex Sequencing of the gDNA of cII from the BigBlue® mouse tissues in accordance with an embodiment of the present technology.

FIGS. 3G and 3H show distribution of mutations identified by direct Duplex Sequencing (FIG. 3G) and among individually collected mutant plaques (FIG. 3H) of cII across all BigBlue® tissue types and treatment groups by codon position and functional consequence, in accordance with an embodiment of the present technology.

FIG. 4 is a bar graph showing mutant frequency measured by Duplex Sequencing in multiple samples of each treatment group and in accordance with an embodiment of the present technology.

FIGS. 5A and 5B are bar graphs showing mutant frequency of endogenous genes as compared to cII transgene in liver (FIG. 5A) and bone marrow (FIG. 5B) and as measured by Duplex Sequencing and in accordance with an embodiment of the present technology.

FIG. 5C is a box plot graph showing SNV mutant frequency (MF) calculated for Duplex Sequencing by genic regions for Liver and Bone Marrow for the indicated treatments categories and in accordance with an embodiment of the present technology.

FIG. 5D is a scatter plot showing individual measurements of aggregate data shown in FIG. 5C in accordance with an embodiment of the present technology.

FIG. 6 is a bar graph showing a mutation spectrum as measured by Duplex Sequencing and in accordance with an embodiment of the present technology.

FIGS. 7A-7C are graphs showing trinucleotide mutation spectra for vehicle control (7A), Benzo[a]pyrene (7B), and N-ethyl-N-nitrosourea (7C) in accordance with an embodiment of the present technology.

FIG. 8 is a bar graph showing mutant frequency of lung, spleen and blood samples for control and experimental animals subjected to urethane in accordance with an embodiment of the present technology.

FIG. 9 is a bar graph showing an average minimum point mutant frequency across groups of tissue samples in accordance with an embodiment of the present technology.

FIG. 10A is a box plot graph showing SNV MF calculated for Duplex Sequencing by genic regions for Lung, Spleen and Blood for the indicated treatments categories and in accordance with an embodiment of the present technology.

FIG. 10B is a scatter plot showing individual measurements of aggregate data shown in FIG. 10A, and in accordance with an embodiment of the present technology.

FIG. 11 is a bar graph showing the mutation spectrum of urethane and a vehicle control within the tested tissues as measured by Duplex Sequencing and in accordance with an embodiment of the present technology.

FIGS. 12A and 12B are graphs showing mutation spectra in the context of adjacent nucleotides (i.e., trinucleotide spectra) for vehicle control (12A), and urethane (12B) in accordance with an embodiment of the present technology.

FIG. 13 shows single nucleotide variant (SNV) spectral strand bias in urethane treated samples in accordance with an embodiment of the present technology.

FIG. 14 is a graph illustrating early stage neoplastic clonal selection of variant allele fractions as detected by Duplex Sequencing in accordance with an embodiment of the present technology.

FIG. 15A is a graph illustrating SNVs plotted over the genomic intervals for the exons captured from the Ras family of genes, including the human transgenic loci, in the Tg-rasH2 mouse model, and in accordance with an embodiment of the present technology.

FIG. 15B is a graph illustrating single nucleotide variants aligning to exon 3 of the human HRAS transgene in accordance with an embodiment of the present technology.

FIGS. 16A-16B are graphical representations of sequencing data from a representative 400 base pair section of human HRAS in mouse lung following urethane treatment using conventional DNA sequencing (FIG. 16A) and Duplex Sequencing (FIG. 16B) in accordance with embodiment of the present technology.

FIGS. 17A-17C are graphs showing mutation spectra in the context of adjacent nucleotides (i.e., trinucleotide spectra) for Signature 1 (FIG. 17A), Signature 4 (FIG. 17B), and Signature 29 (FIG. 17C) from COSMIC.

FIG. 18 shows unsupervised hierarchical clustering of all 30 published COSMIC signatures and the 4 cohort spectra from Examples 1 and 2 in accordance with an embodiment of the present technology.

FIG. 19 is a schematic diagram of a network computer system for use with the methods and/or kits disclosed herein to identify mutagenic events and/or nucleic acid damage events resulting from genotoxic exposure in accordance with an embodiment of the present technology.

FIG. 20 is a flow diagram illustrating a routine for providing Duplex Sequencing consensus sequence data in accordance with an embodiment of the present technology in accordance with an embodiment of the present technology.

FIG. 21 is a flow diagram illustrating a routine for detecting and identifying mutagenic events resulting from genotoxic exposure of a sample in accordance with an embodiment of the present technology.

FIG. 22 is a flow diagram illustrating a routine for detecting and identifying DNA damage events resulting from genotoxic exposure of a sample in accordance with an embodiment of the present technology.

FIG. 23 is a flow diagram illustrating a routine for detecting and identifying a carcinogen or carcinogen exposure in a subject in accordance with an embodiment of the present technology.

### DETAILED DESCRIPTION

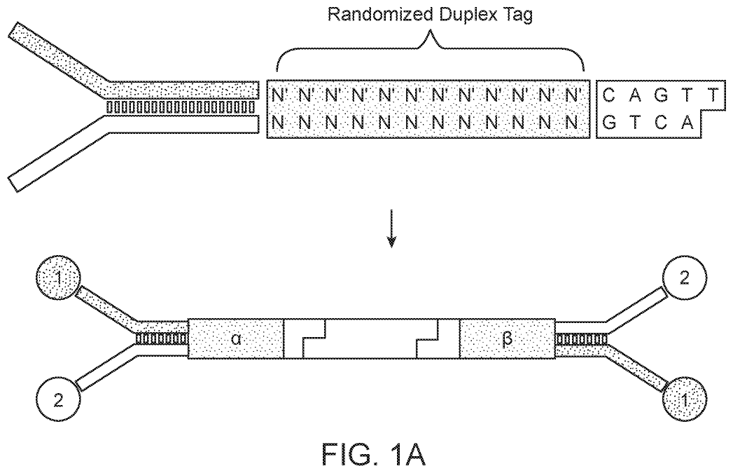

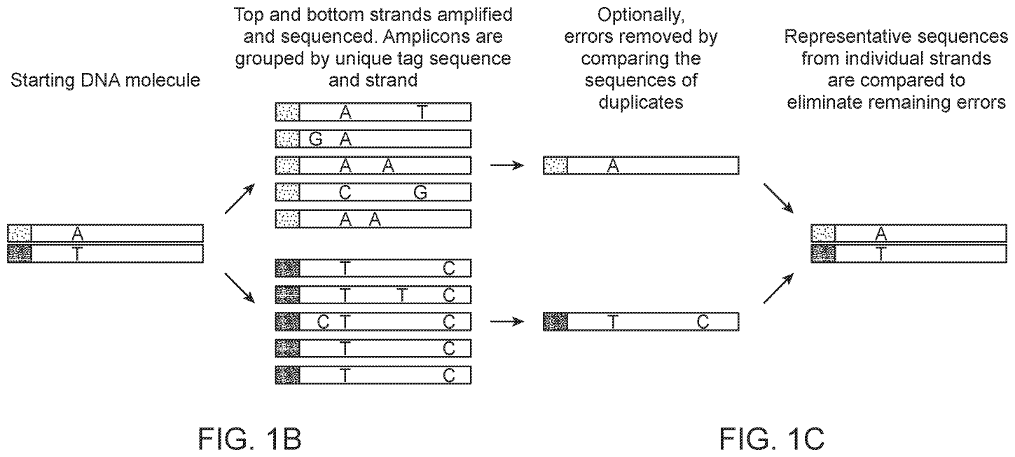

Specific details of several embodiments of the technology are described below with reference to FIGS. 1A-20. The embodiments can include, for example, methods, systems, kits, etc. for assessing genotoxicity. Some embodiments of the technology are directed to utilizing Duplex Sequencing for assessing a genotoxic potential of an agent (e.g., a chemical compound) or any other type of exposure (e.g., a radiation source) in an exposed subject, model organism or model cell culture system. Other embodiments of the technology are directed to utilizing Duplex Sequencing for determining a mutation signature associated with a genotoxic agent. Additional embodiments of the technology are directed to identifying one or more genotoxic agents a subject may have been exposed to by comparing the subject's DNA mutation spectrum with mutation spectra of known mutagenic compounds. Additional embodiments of the technology are directed to identifying one or more locations or environments a subject may have been exposed to by comparing the subject's DNA mutation spectrum from one or more cell types in one or more tissues with mutation spectra of known environments or compounds known to be present in such locations or environments. Additional embodiments of the technology are directed to identifying a subject by comparing the subject's DNA mutation spectrum from one or more cell types in one or more tissues with mutation spectra of known individuals or of locations or environments the individual has known to have been exposed to or compounds known to be present in such locations or environments. In certain embodiments, a genotoxin can be assessed for carcinogenic potential. Additional embodiments include identifying and assessing carcinogenesis risk resulting from either mutagenic or non-mutagenic carcinogens by identifying mutation-bearing clones that are emerging with cancer driver mutations. Additional embodiments include identifying and assessing carcinogenesis risk resulting from either mutagenic or non-mutagenic carcinogens by identifying emergency of mutation-bearing clones where the mutations are not believed to be cancer drivers (often known as “passenger” or “hitchhiker” mutations) but substantially uniquely mark clones (Salk and Horwitz Sem Cancer Bio 2010 PMID: 20951806) Other embodiments of the technology are directed to utilizing Duplex Sequencing for detecting and assessing nucleic acid damage (particularly DNA damage such as adducts) resulting from genotoxin exposure or other endogenous genotoxic processes (e.g., aging).

Although many of the embodiments are described herein with respect to Duplex Sequencing, other sequencing modalities capable of generating error-corrected sequencing reads in addition to those described herein are within the scope of the present technology. Additionally, other embodiments of the present technology can have different configurations, components, or procedures than those described herein. A person of ordinary skill in the art, therefore, will accordingly understand that the technology can have other embodiments with additional elements and that the technology can have other embodiments without several of the features shown and described below with reference to FIGS. 1A-20.

### Definitions

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Where ranges are provided herein, the endpoints are included. As used in this application, the term “comprise” and variations of the term, such as “comprising” and “comprises,” are not intended to exclude other additives, components, integers or steps.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value. For variances of single digit integer values where a single numerical value step in either the positive or negative direction would exceed 25% of the value, “about” is generally accepted by those skilled in the art to include, at least 1, 2, 3, 4, or 5 integer values in either the positive or negative direction, which may or may not cross zero depending on the circumstances. A non-limiting example of this is the supposition that 3 cents can be considered about 5 cents in some situations that would be apparent to one skilled in that art.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Biological Sample: As used herein, the term “biological sample” or “sample” typically refers to a sample obtained or derived from a biological source (e.g., a tissue or organism or cell culture) of interest, as described herein. In some embodiments, a source of interest comprises an organism, such as an animal or human. In other embodiments, a source of interest comprises a microorganism, such as a bacterium, virus, protozoan, or fungus. In further embodiments, a source of interest may be a synthetic tissue, organism, cell culture, nucleic acid or other material. In yet further embodiments, a source of interest may be a plant-based organism. In yet another embodiment, a sample may be an environmental sample such as, for example, a water sample, soil sample, archeological sample, or other sample collected from a non-living source. In other embodiments, a sample may be a multi-organism sample (e.g., a mixed organism sample). In some embodiments, a biological sample is or comprises biological tissue or fluid. In some embodiments, a biological sample may be or comprise bone marrow; blood; blood cells; ascites; tissue samples, biopsy samples or or fine needle aspiration samples; cell-containing body fluids; free floating nucleic acids; protein-bound nucleic acids, riboprotein-bound nucleic acids; sputum; saliva; urine; cerebrospinal fluid, peritoneal fluid; pleural fluid; feces; lymph; gynecological fluids; skin swabs; vaginal swabs; pap smear, oral swabs; nasal swabs; washings or lavages such as a ductal lavages or broncheoalveolar lavages; vaginal fluid, aspirates; scrapings; bone marrow specimens; tissue biopsy specimens; fetal tissue or fluids; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments cell-derivatives such as organelles or vesicles or exosomes. In a particular embodiment, a biological sample is a liquid biopsy obtained from a subject. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cancer disease: In an embodiment, the genotoxic associated disease or disorder is a “cancer disease” which is familiar to those experience in the art as being generally characterized by dysregulated growth of abnormal cells, which may metastasize. Cancer diseases detectable using one or more aspects of the present technology comprise, by way of non-limiting examples, prostate cancer (i.e. adenocarcinoma, small cell), ovarian cancer (e.g., ovarian adenocarcinoma, serous carcinoma or embryonal carcinoma, yolk sac tumor, teratoma), liver cancer (e.g., HCC or hepatoma, angiosarcoma), plasma cell tumors (e.g., multiple myeloma, plasmacytic leukemia, plasmacytoma, amyloidosis, Waldenstrom's macroglobulinemia), colorectal cancer (e.g., colonic adenocarcinoma, colonic mucinous adenocarcinoma, carcinoid, lymphoma and rectal adenocarcinoma, rectal squamous carcinoma), leukemia (e.g., acute myeloid leukemia, acute lymphocytic leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia, T-cell leukemia, Sezary syndrome, systemic mastocytosis, hairy cell leukemia, chronic myeloid leukemia blast crisis), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, follicular lymphoma, mantle cell lymphoma, MALT lymphoma, marginal cell lymphoma, Richter's transformation, double hit lymphoma, transplant associated lymphoma, CNS lymphoma, extranodal lymphoma, HIV-associated lymphoma, endemic lymphoma, Burkitt's lymphoma, transplant-associated lymphoproliferative neoplasms, and lymphocytic lymphoma etc.), cervical cancer (squamous cervical carcinoma, clear cell carcinoma, HPV associated carcinoma, cervical sarcoma etc.) esophageal cancer (esophageal squamous cell carcinoma, adenocarcinoma, certain grades of Barretts esophagus, esophageal adenocarcinoma), melanoma (dermal melanoma, uveal melanoma, acral melanoma, amelanotic melanoma etc.), CNS tumors (e.g., oligodendroglioma, astrocytoma, glioblastoma multiforme, meningioma, schwannoma, craniopharyngioma etc.), pancreatic cancer (e.g., adenocarcinoma, adenosquamous carcinoma, signet ring cell carcinoma, hepatoid carcinoma, colloid carcinoma, islet cell carcinoma, pancreatic neuroendocrine carcinoma etc.), gastrointestinal stromal tumor, sarcoma (e.g., fibrosarcoma, myxosarcoma, liposarcoma, chondrosarcoma, osteogenic sarcoma, angiosarcoma, endothelioma sarcoma, lymphangiosarcoma, lymphangioendothelioma sarcoma, leiomyosarcoma, Ewing's sarcoma, and rhabdomyo sarcoma, spindle cell tumor etc.), breast cancer (e.g., inflammatory carcinoma, lobar carcinoma, ductal carcinoma etc.), ER-positive cancer, HER-2 positive cancer, bladder cancer (squamous bladder cancer, small cell bladder cancer, urothelial cancer etc.), head and neck cancer (e.g., squamous cell carcinoma of the head and neck, HPV-associated squamous cell carcinoma, nasopharyngeal carcinoma etc.), lung cancer (e.g., non-small cell lung carcinoma, large cell carcinoma, bronchogenic carcinoma, squamous cell cancer, small cell lung cancer etc.), metastatic cancer, oral cavity cancer, uterine cancer (leiomyosarcoma, leiomyoma etc.), testicular cancer (e.g., seminoma, non-seminoma, and embryonal carcinoma yolk sack tumor etc), skin cancer (e.g., squamous cell carcinoma, and basal cell carcinoma, merkel cell carcinoma, melanoma, cutaneous t-cell lymphoma etc.), thyroid cancer (e.g., papillary carcinoma, medullary carcinoma, anaplastic thyroid cancer etc.), stomach cancer, intra-epithelial cancer, bone cancer, biliary tract cancer, eye cancer, larynx cancer, kidney cancer (e.g., renal cell carcinoma, Wilms tumor etc.), gastric cancer, blastoma (e.g., nephroblastoma, medulloblastoma, hemangioblastoma, neuroblastoma, retinoblastoma, etc.), myeloproliferative neoplasms (polycythemia vera, essential thrombocytosis, myelofibrosis, etc.), chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, pheochromocytoma, small cell lung cancer, peritoneal mesothelioma, hyperparathyroid adenoma, adrenal cancer, cancer of unknown primary, cancer of the endocrine system, cancer of the penis, cancer of the urethra, cutaneous or intraocular melanoma, a gynecologic tumor, solid tumors of childhood, or neoplasms of the central nervous system, primary mediastinal germ cell tumor, clonal hematopoiesis of indeterminate potential, smoldering myeloma, monoclonal gammaglobulinopathy of unknown significant, monoclonal B-cell lymphocytosis, low grade cancers, clonal field defects, preneoplastic neoplasms, ureteral cancer, autoimmune-associated cancers (i.e. ulcerative colitis, primary sclerosing cholangitis, celiac disease), cancers associated with an inherited predisposition (i.e. those carrying genetic defects in such as BRCA1, BRCA2, TP53, PTEN, ATM, etc.) and various genetic syndromes such as MEN1, MEN2 trisomy 21 etc.) and those occurring when exposed to chemicals in utero (i.e. clear cell cancer in female offspring of women exposed to Diethylstilbestrol [DES]), among many others.

Cancer driver or Cancer driver gene: As used herein, “cancer driver” or “cancer driver gene” refers to a genetic lesion that has the potential to allow a cell, in the right context, to undergo malignant transformation. Such genes include tumor suppressors (e.g., TP53, BRCA1) that normally suppress malignancy transformation and when mutated in certain ways, no longer do. Other driver genes can be oncogenes (e.g., KRAS, EGFR) that when mutated in certain ways become constitutively active or gain new properties that facilitate a cell to become malignant. Other mutations found in non-coding regions of the genome can be cancer drivers. For example, a mutation of the promoter region of the telomerase gene (TERT) can result in overexpression of the gene and thus become a cancer driver. Certain rearrangements (e.g., BCR-ABL fusion) can juxtapose one genetic region with that of another to drive tumorigenesis through mechanisms related to overexpression, loss of repression or chimeric fusion genes. Broadly speaking, genetic mutations (or epimutations) that confer a phenotype to a cell that facilitates its proliferation, survival or competitive advantage over other cells or that renders its ability to evolve more robust, can be considered a driver mutation. This is to be contrasted with mutations that lack such features, even if they may happen to be in the same gene (i.e. a synonymous mutation). When such mutations are identified in tumors, they are commonly referred to as passenger mutations because they “hitchhiked” along with the clonal expansion without meaningfully contributing to the expansion. As recognized by one or ordinary skill in the art, the distinction of driver and passenger is not absolute and should not be construed as such. Some drivers only function in certain situations (e.g., certain tissues) and others may not operate in the absence of other mutations or epimutations or other factors.

Control sample: As used herein, a “control sample” refers to a sample isolated in the same way as the sample to which it is compared, except that the control sample is not exposed to an agent, environment or process being evaluated for genotoxic potential.

Determine: Many methodologies described herein include a step of “determining” Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Duplex Sequencing (DS): As used herein, “Duplex Sequencing (DS)” is, in its broadest sense, refers to a tag-based error-correction method that achieves exceptional accuracy by comparing the sequence from both strands of individual DNA molecules.

Genotoxicity: As used herein, the term “genotoxicity” refers to the destructive property of agents or processes (i.e., genotoxins) that cause damage to genetic material (e.g., DNA, RNA). Polynucleotide damage, formation of a genetic mutation and/or the disruption of normal nucleic acid structure resulting directly or indirectly from exposure to a genotoxin are aspects of genotoxicity. A subject exposed to a genotoxin may potentially develop a disease or disorder (e.g. cancer) immediately or years later. In an embodiment, the present technology is directed in part to identifying contributing events and/or factors (e.g., agents, processes) causing genotoxicity in a subject in order to prevent or reduce the risk of the disease or disorder onset, and/or counter the adverse effects thereof. In other embodiments, initiating genotoxicity is by design, such as for creating diversity in a genetic library.

Genotoxin or Genotoxic agent or factor: As used herein, the term “genotoxin” or “genotoxic agent or factor” refers to, for example, any chemical that a nucleic acid source (e.g., biological source, subject) is exposed to and/or consumes, environmental exposures, and/or any triggering event (endogenous precursor mutation) that causes polynucleotide damage, a genomic mutation or the disruption of normal nucleic acid structure. In some embodiments, a genotoxin has the ability to directly or indirectly (e.g. triggers a mutagenic precursor), or both, cause a disease or disorder development in a subject. Genotoxic factors or agents that are able to be detected by the present technology comprise, by way of non-limiting examples, a chemical or a mixture of chemicals (e.g. pharmaceuticals, industrial additives and byproducts-waste, petroleum distillates, heavy metals, cosmetics, household cleaners, airborne particulates, food products, byproducts of manufacturing, contaminants, plasticizers, detergents, etc.); and radiation (particle radiation, photons, or both) and/or physical forces (e.g. a magnetic field, gravitational field, acceleration forces, etc.) generated by the natural environment or manmade (e g from a device). The genotoxin may further comprise a liquid, solid, and/or an aerosol formulation and exposure thereof may be via any route of administration. A genotoxic agent or factor may be exogenous (e.g., exposure originates from outside the biological source, or in other instances, the genotoxic agent or factor may be endogenous to the biological source, or a combination thereof. An exogenously originating agent or factor may become genotoxic once such exposure is processed endogenously. In still other examples, an agent or factor may become genotoxic when combined with one or more additional agents or factors, and may, in some instances have a synergistic effect. Additional examples of genotoxic factors or agents may further include an organism capable of, directly or indirectly, causing nucleic acid damage in a subject upon exposure (e.g. via infection of the subject), such as by way of non-limiting examples, schistosomiasis contributing to bladder cancer, HPV contributing to cervical or head and neck cancer, polyomavirus contributing to Merkel cell carcinoma, Helicobacter pylori contributing to gastric cancer, chronic bacterial infection of a skin wound contributing to squamous cell carcinoma, etc. Additional genotoxic agents or factors may further include an organism able to produce (e.g. within itself or to secrete) a genotoxic agent, such as by way of non-limiting examples, aflatoxin from Aspergillus flavus, or aristolochic acid from the aristocholia family of plants, etc. Genotoxic factors or agents that are able to be detected using various aspects of the present technology may further comprise endogenous genotoxins, which may not be able to be precisely quantified or experimentally controlled, such as by way of non-limiting examples, stress, inflammation, effects of therapy treatments (e.g. gene therapy, gene editing therapy, stem cell therapy, other cellular therapies, a pharmaceutical, radiography, etc.). Endogenous factors may also represent the aggregate accumulation of mutations and other genotoxic events in the tissues of a subject that reflect the integral effects of the subject's exposures.

Genotoxic associated disease or disorder: As used herein, the term “genotoxic-associated disease or disorder” refers to any medical condition resulting from a genomic mutation or other polynucleotide damage or rearrangement in a subject that is directly or indirectly caused by exposure to one or more genotoxins. A genotoxic-associated disease or disorder may be cancer-related or non-cancer-related. Additionally, the polynucleotide damage/rearrangement or mutation can be in a germ cell or somatic cell. In examples, where a germ cell is affected, it is contemplated that genotoxic-associated disease or disorder may manifest in (or otherwise confer a risk to) a subject that is a progeny of an exposed subject.

Sufficiently genotoxic agent: As used herein, the term “sufficiently genotoxic agent” refers to an agent, factor, compound or process identified by the system, methods and kits of the present technology to have an about 50%, about 40%, about 30%, about 20%, about 10%, about 5%, about 4%, about 3%, about 2%, about 1%, about 0.5%, about 0.1%, about 0.01%, about 0.001%, about 0.0001%, about 0.00001%, about 0.000001% etc. probability of causing nucleic acid damage or mutation at one or more nucleotide residues in one or more molecules that may derive from one or more biological organisms having been exposed. In some embodiments, a sufficiently genotoxic agent can have more than about a 50% probability of causing nucleic acid damage or mutation that above a control background level. In some embodiments, a sufficiently genotoxic agent refers to an agent, factor, compound or process identified by the system, methods and kits of the present technology to have an about 50%, about 40%, about 30%, about 20%, about 10%, about 5%, about 4%, about 3%, about 2%, about 1%, about 0.5%, about 0.1%, about 0.01%, about 0.001%, about 0.0001%, about 0.00001% etc. probability of causing a disease or disorder in a subject exposed to the genotoxin.

Inhibit growth: As used herein, the term to “inhibit growth” in a cancer disease refers to causing a reduction in cell growth (e.g., tumor size, cancer cell rate of division etc) in vivo or in vitro by, e.g., about 5%, about 10%, about 20%, about 30%, about 40%, about 50%, about 60%, about 70%, about 80%, about 90%, about 95%, or about 99% or more, as evident by a reduction in the proliferation of cells and/or the size/mass of cells exposed to a treatment relative to the proliferation and/or cell size growth of cells in the absence of the treatment. Growth inhibition may be the result of a treatment that induces apoptosis in a cell, induces necrosis in a cell, slows cell cycle progression, disrupts cellular metabolism, induces cell lysis, or induces some other mechanism that reduces the proliferation and/or cell size growth of cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Mechanism of Action: As used herein, the term “mechanism of action” refers to the biochemical process that results in alteration to nucleic acid following exposure to a genotoxin. In an embodiment, the “mechanism of action” refers to the the biochemical pathway and or pathophysiological processes that follow the genomic mutation or damage until full onset of the disease or disorder. In another embodiment, the “mechanism of action” includes the biochemical pathway and/or physiological processes that occur in a biological source following genotoxin exposure and which results in genomic damage (e.g. premutagenic lesions) or mutation. In yet another embodiment, the mechanism of action of a genotoxic agent or process may be inferred from one or more of the following: the nucleotide base affected, the nucleotide change introduced, the type of DNA damage introduced, the structural change introduced, the flanking nucleotide sequence context of the nucleotide(s) affected, the genetic context or the sequence(s) affected, the transcriptional status or the region affected, the methylation status of the region affected, the protein bound status or condensation status or chromosome location of the region affected by the genotoxin exposure.

Mutation: As used herein, the term “mutation” refers to alterations to nucleic acid sequence or structure. Mutations to a polynucleotide sequence can include point mutations (e.g., single base mutations), multinucleotide mutations, nucleotide deletions, sequence rearrangements, nucleotide insertions, and duplications of the DNA sequence in the sample, among complex multinucelotide changes Mutations can occur on both strands of a duplex DNA molecule as complementary base changes (i.e. true mutations), or as a mutation on one strand but not the other strand (i.e. heteroduplex), that has the potential to be either repaired, destroyed or be mis-repaired/converted into a true double stranded mutation.

Mutant frequency: As used herein, the term “mutant frequency”, also sometimes referred to as “mutant frequency”, refers to the number of unique mutations detected per the total number of duplex base-pairs sequenced. In some embodiments, the mutant frequency is the frequency of mutations within only a specific gene, or a set of genes or a set of genomic targets. In some embodiments mutant frequency may refer to only certain types of mutations (for example the frequency of A>T mutations, which is calculated as the number of A>T mutations per the total number of A bases) The frequency at which mutations are introduced into a population of cells or molecules can vary by genotoxin, by amount of time or level of exposure to a genotoxin, by age of a subject, over time, by tissue or organization type, by region of a genome, by type of mutation, by trinucleotide context, inherited genetic background among other things.

Mutation signature: As used herein, the term “mutation signature” and “mutation spectrum or spectra” refers to characteristic combinations of mutation types arising from mutagenesis processes such as DNA replication infidelity, exogenous and endogenous genotoxin exposures, defective DNA repair pathways and DNA enzymatic editing. In an embodiment, the mutation spectrum is generated by computational pattern matching (e.g., unsupervised hierarchical mutation spectrum clustering).

Non-cancerous disease: In another embodiment, the genotoxic associated disease or disorder is a non-cancerous disease; instead it is yet another type of disease or disorder caused by, or contributed to by, a genomic mutation or damage. By way of non-limiting examples, such non-cancerous types of diseases or disorders that are detectable or predicted using one or more aspects of the present technology comprise diabetes; autoimmune disease or disorders, infertility, neurodegeneration, progeria, cardiovascular disease, any disease associated with treatment for another genetically-mediated disease (i.e. chemotherapy-mediated neuropathy and renal failure associated with chemotherapy such as cisplatin), Alzheimer's/dementia, obesity, heart disease, high blood pressure, arthritis, mental illness, other neurological disorders (neurofibromatosis), and a multifactorial inheritance disorder (e.g., a predisposition triggered by environmental factors).

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present technology. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double-stranded. In some embodiments a nucleic acid may be branched of have secondary structures. In some embodiments a nucleic acid has a nucleotide sequence comprising at least one element that encodes, or is the complement of a sequence that encodes, a polypeptide. In some embodiments, a nucleic acid has enzymatic activity. In some embodiments the nucleic acid serves a mechanical function, for example in a ribonucleoprotein complex or a transfer RNA.

Pharmaceutical composition or formulation: As used herein, the term “pharmaceutical composition” comprises a pharmacologically effective amount of an active drug or active agent and a pharmaceutically acceptable carrier. In some examples, various aspects of the present technology can be used to assess the genotoxicity of the pharmaceutical composition or formulation, or the active drug or agent therein.

Polynucleotide damage: As used herein, the term “polynucleotide damage” or “nucleic acid damage” refers to damage to a subject's deoxyribonucleic acid (DNA) sequence (“DNA damage”) or ribonucleic acid (RNA) sequence (“RNA damage”) that is directly or indirectly (e.g. a metabolite, or induction of a process that is damaging or mutagenic) caused by a genotoxin. Damaged nucleic acid may lead to the onset of a disease or disorder associated with genotoxin exposure in a subject. In some embodiments, detection of damaged nucleic acid in a subject may be an indication of a genotoxin exposure. Polynucleotide damage may further comprise chemical and/or physical modification of the DNA in a cell. In some embodiments, the damage is or comprises, by way of non-limiting examples, at least one of oxidation, alkylation, deamination, methylation, hydrolysis, hydroxylation, nicking, intra-strand crosslinks, inter-strand cross links, blunt end strand breakage, staggered end double strand breakage, phosphorylation, dephosphorylation, sumoylation, glycosylation, deglycosylation, putrescinylation, carboxylation, halogenation, formylation, single-stranded gaps, damage from heat, damage from desiccation, damage from UV exposure, damage from gamma radiation damage from X-radiation, damage from ionizing radiation, damage from non-ionizing radiation, damage from heavy particle radiation, damage from nuclear decay, damage from beta-radiation, damage from alpha radiation, damage from neutron radiation, damage from proton radiation, damage from cosmic radiation, damage from high pH, damage from low pH, damage from reactive oxidative species, damage from free radicals, damage from peroxide, damage from hypochlorite, damage from tissue fixation such formalin or formaldehyde, damage from reactive iron, damage from low ionic conditions, damage from high ionic conditions, damage from unbuffered conditions, damage from nucleases, damage from environmental exposure, damage from fire, damage from mechanical stress, damage from enzymatic degradation, damage from microorganisms, damage from preparative mechanical shearing, damage from preparative enzymatic fragmentation, damage having naturally occurred in vivo, damage having occurred during nucleic acid extraction, damage having occurred during sequencing library preparation, damage having been introduced by a polymerase, damage having been introduced during nucleic acid repair, damage having occurred during nucleic acid end-tailing, damage having occurred during nucleic acid ligation, damage having occurred during sequencing, damage having occurred from mechanical handling of DNA, damage having occurred during passage through a nanopore, damage having occurred as part of aging in an organism, damage having occurred as a result if chemical exposure of an individual, damage having occurred by a mutagen, damage having occurred by a carcinogen, damage having occurred by a clastogen, damage having occurred from in vivo inflammation damage due to oxygen exposure, damage due to one or more strand breaks, and any combination thereof.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value or representation thereof in a physical or computer database that may be present at a location or accessed remotely via electronic means. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. A “reference sample” refers to a sample from a subject that is distinct from the test subject and isolated in the same way as the sample to which it is compared, and which has been exposed to a known quantity of the same genotoxic agent. The subject of the reference sample may be genetically identical to the test subject or may be different. In addition, the reference sample may be derived from several subjects who have been exposed to a known quantity of the same genotoxic agent.

Safe threshold level: As used herein, the term “safe threshold level” refers to the amount (e.g. weight, volume, concentration, mass, molar abundance, unit*time integrals etc.) of a specific genotoxin or a combination of genotoxins a subject may be exposed to before a likely genomic mutation occurs leading to disease onset. For example, a safe threshold level may be zero. In other examples, a level of genotoxin exposure may be tolerable. Toleration of acceptable risk of exposure may differ depending on subject, age, gender, tissue type, health condition of the patient, and other risk-benefit considerations familiar to one experienced in the art etc.

Safe threshold mutant frequency: As used herein, the term “safe threshold mutant frequency” refers to an acceptable rate of mutation caused by a genotoxic agent or process, below which a subject assumes an acceptable risk of acquiring a genotoxic-associated disease or disorder. Toleration of acceptable risk of exposure and resultant mutation rate may differ depending on subject, age, gender, tissue type, health condition of the patient, etc.

Single Molecule Identifier (SMI): As used herein, the term “single molecule identifier” or “SMI”, (which may be referred to as a “tag” a “barcode”, a “molecular bar code”, a “Unique Molecular Identifier”, or “UMI”, among other names) refers to any material (e.g., a nucleotide sequence, a nucleic acid molecule feature) that is capable of substantially distinguishing an individual molecule among a larger heterogeneous population of molecules. In some embodiments, a SMI can be or comprise an exogenously applied SMI. In some embodiments, an exogenously applied SMI may be or comprise a degenerate or semi-degenerate sequence. In some embodiments substantially degenerate SMIs may be known as Random Unique Molecular Identifiers (R-UMIs). In some embodiments an SMI may comprise a code (for example a nucleic acid sequence) from within a pool of known codes. In some embodiments pre-defined SMI codes are known as Defined Unique Molecular Identifiers (D-UMIs). In some embodiments, a SMI can be or comprise an endogenous SMI. In some embodiments, an endogenous SMI may be or comprise information related to specific shear-points of a target sequence, features relating to the terminal ends of individual molecules comprising a target sequence, or a specific sequence at or adjacent to or within a known distance from an end of individual molecules. In some embodiments an SMI may relate to a sequence variation in a nucleic acid molecule cause by random or semi-random damage, chemical modification, enzymatic modification or other modification to the nucleic acid molecule. In some embodiments the modification may be deamination of methylcytosine. In some embodiments the modification may entail sites of nucleic acid nicks. In some embodiments, an SMI may comprise both exogenous and endogenous elements. In some embodiments an SMI may comprise physically adjacent SMI elements. In some embodiments SMI elements may be spatially distinct in a molecule. In some embodiments an SMI may be a non-nucleic acid. In some embodiments an SMI may comprise two or more different types of SMI information. Various embodiments of SMIs are further disclosed in International Patent Publication No. WO2017/100441, which is incorporated by reference herein in its entirety.

Strand Defining Element (SDE): As used herein, the term “Strand Defining Element” or “SDE”, refers to any material which allows for the identification of a specific strand of a double-stranded nucleic acid material and thus differentiation from the other/complementary strand (e.g., any material that renders the amplification products of each of the two single stranded nucleic acids resulting from a target double-stranded nucleic acid substantially distinguishable from each other after sequencing or other nucleic acid interrogation). In some embodiments, a SDE may be or comprise one or more segments of substantially non-complementary sequence within an adapter sequence. In particular embodiments, a segment of substantially non-complementary sequence within an adapter sequence can be provided by an adapter molecule comprising a Y-shape or a “loop” shape. In other embodiments, a segment of substantially non-complementary sequence within an adapter sequence may form an unpaired “bubble” in the middle of adjacent complementary sequences within an adapter sequence. In other embodiments an SDE may encompass a nucleic acid modification. In some embodiments an SDE may comprise physical separation of paired strands into physically separated reaction compartments. In some embodiments an SDE may comprise a chemical modification. In some embodiments an SDE may comprise a modified nucleic acid. In some embodiments an SDE may relate to a sequence variation in a nucleic acid molecule caused by random or semi-random damage, chemical modification, enzymatic modification or other modification to the nucleic acid molecule. In some embodiments the modification may be deamination of methylcytosine. In some embodiments the modification may entail sites of nucleic acid nicks. Various embodiments of SDEs are further disclosed in International Patent Publication No. WO2017/100441, which is incorporated by reference herein in its entirety.

Subject: As used herein, the term “subject” refers an organism, typically a mammal, such as a human (in some embodiments including prenatal human forms), a non-human animal (e.g., mammals and non-mammals including, but not limited to, non-human primates, horses, sheep, dogs, cows, pigs, chickens, amphibians, reptiles, sea-life (generally excluding sea monkeys), other model organisms such as worms, flys etc.), and transgenic animals (e.g., transgenic rodents), etc. In some embodiments, a subject has been exposed to genotoxin or genotoxic factor or agent, or in another embodiment, the subject has been exposed to a potential genotoxin. In some embodiments, a subject is suffering from a relevant disease, disorder or condition. In some embodiments, a subject is suffering from a genotoxic associated disease or disorder. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is displaying a symptom or characteristic of a disease, disorder, or condition, and in some embodiments, such symptom or characteristic is associated with a genotoxic associated disease or disorder. In some embodiments, a subject is a patient. In some embodiments, a subject is an individual to whom diagnosis and/or therapy is and/or has been administered. In still other embodiments, a subject refers to any living biological sources or other nucleic acid material, that can be exposed to genotoxins, and can include, for example, organisms, cells, and/or tissues, such as for in vivo studies, e.g.: fungi, protozoans, bacteria, archaebacteria, viruses, isolated cells in culture, cells that have been intentionally (e.g., stem cell transplant, organ transplant) or unintentionally (i.e. fetal or maternal microchimerism) or isolated nucleic acids or organelles (i.e. mitochondria, chloroplasts, free viral genomes, free plasmids, aptamers, ribozymes or derivatives or precursors of nucleic acids (i.e. oligonucleotides, dinucleotide triphosphates, etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” or “pharmacologically effective amount” or simply “effective amount” refers to that amount of an active drug or agent to produce an intended pharmacological, therapeutic, or preventive result. In some examples, various aspects of the present technology can be used to assess or determine a effective amount of an active drug or agent (e.g., an active drug delivered to purposefully induce genotoxicity-associated events).

Trinucleotide or trinucleotide context: As used herein, the terms “trinucleotide” or “trinucleotide context” refers to a nucleotide within the context of nucleotide bases immediately preceding and immediately following in sequence (e.g., a mononucleotide within a three-mononucleotide combination).

Trinucleotide spectrum or signature: Herein, the term “trinucleotide signature” is used interchangeably with “trinucleotide spectrum”, “triplet signature” and “triplet spectrum” refers to a mutation signature, such as those associated with a genotoxin exposure, in a trinucleotide context. In one embodiment, a genotoxin can have a unique, semi-unique and/or otherwise identifiable triplet spectrum/signature.

Treatment: As used herein, the term “treatment” refers to the application or administration of a therapeutic agent to a subject, or application or administration of a therapeutic agent to an isolated tissue or cell line from a subject, who has a disorder, e.g., a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. In one example, the disorder or disease/condition is a genotoxic disease or disorder. In another example, the disorder or disease/condition is not a genotoxic disease or disorder. In some examples, various aspects of the present technology are used to assess the genotoxicity of the treatment or a potential treatment.

### Selected Embodiments of Duplex Sequencing Methods and Associated Adapters and Reagents

Duplex Sequencing is a method for producing error-corrected DNA sequences from double stranded nucleic acid molecules, and which was originally described in International Patent Publication No. WO 2013/142389 and in U.S. Pat. No. 9,752,188, and WO 2017/100441, in Schmitt et. al., PNAS, 2012 [1]; in Kennedy et. al., PLOS Genetics, 2013 [2]; in Kennedy et. al., Nature Protocols, 2014 [3]; and in Schmitt et. al., Nature Methods, 2015 [4]. Each of the above-mentioned patents, patent applications and publications are incorporated herein by reference in their entireties. As illustrated in FIGS. 1A-1C, and in certain aspects of the technology, Duplex Sequencing can be used to independently sequence both strands of individual DNA molecules in such a way that the derivative sequence reads can be recognized as having originated from the same double-stranded nucleic acid parent molecule during massively parallel sequencing (MPS), also commonly known as next generation sequencing (NGS), but also differentiated from each other as distinguishable entities following sequencing. The resulting sequence reads from each strand are then compared for the purpose of obtaining an error-corrected sequence of the original double-stranded nucleic acid molecule known as a Duplex Consensus Sequence (DCS). The process of Duplex Sequencing makes it possible to explicitly confirm that both strands of an original double stranded nucleic acid molecule are represented in the generated sequencing data used to form a DCS.

In certain embodiments, methods incorporating DS may include ligation of one or more sequencing adapters to a target double-stranded nucleic acid molecule, comprising a first strand target nucleic acid sequence and a second strand target nucleic sequence, to produce a double-stranded target nucleic acid complex (e.g. FIG. 1A).

In various embodiments, a resulting target nucleic acid complex can include at least one SMI sequence, which may entail an exogenously applied degenerate or semi-degenerate sequence (e.g., randomized duplex tag shown in FIG. 1A, sequences identified as α and β in FIG. 1A), endogenous information related to the specific shear-points of the target double-stranded nucleic acid molecule, or a combination thereof. The SMI can render the target-nucleic acid molecule substantially distinguishable from the plurality of other molecules in a population being sequenced either alone or in combination with distinguishing elements of the nucleic acid fragments to which they were ligated. The SMI element's substantially distinguishable feature can be independently carried by each of the single strands that form the double-stranded nucleic acid molecule such that the derivative amplification products of each strand can be recognized as having come from the same original substantially unique double-stranded nucleic acid molecule after sequencing. In other embodiments the SMI may include additional information and/or may be used in other methods for which such molecule distinguishing functionality is useful, such as those described in the above-referenced publications. In another embodiment, the SMI element may be incorporated after adapter ligation. In some embodiments the SMI is double-stranded in nature. In other embodiments it is single-stranded in nature (e.g., the SMI can be on the single-stranded portion(s) of the adapters). In other embodiments it is a combination of single-stranded and double-stranded in nature.

In some embodiments, each double-stranded target nucleic acid sequence complex can further include an element (e.g., an SDE) that renders the amplification products of the two single-stranded nucleic acids that form the target double-stranded nucleic acid molecule substantially distinguishable from each other after sequencing. In one embodiment, an SDE may comprise asymmetric primer sites comprised within the sequencing adapters, or, in other arrangements, sequence asymmetries may be introduced into the adapter molecules not within the primer sequences, such that at least one position in the nucleotide sequences of the first strand target nucleic acid sequence complex and the second stand of the target nucleic acid sequence complex are different from each other following amplification and sequencing. In other embodiments, the SMI may comprise another biochemical asymmetry between the two strands that differs from the canonical nucleotide sequences A, T, C, G or U, but is converted into at least one canonical nucleotide sequence difference in the two amplified and sequenced molecules. In yet another embodiment, the SDE may be a means of physically separating the two strands before amplification, such that the derivative amplification products from the first strand target nucleic acid sequence and the second strand target nucleic acid sequence are maintained in substantial physical isolation from one another for the purposes of maintaining a distinction between the two. Other such arrangements or methodologies for providing an SDE function that allows for distinguishing the first and second strands may be utilized, such as those described in the above-referenced publications, or other methods that serves the functional purpose described.

After generating the double-stranded target nucleic acid complex comprising at least one SMI and at least one SDE, or where one or both of these elements will be subsequently introduced, the complex can be subjected to DNA amplification, such as with PCR, or any other biochemical method of DNA amplification (e.g., rolling circle amplification, multiple displacement amplification, isothermal amplification, bridge amplification or surface-bound amplification, such that one or more copies of the first strand target nucleic acid sequence and one or more copies of the second strand target nucleic acid sequence are produced (e.g., FIG. 1B). The one or more amplification copies of the first strand target nucleic acid molecule and the one or more amplification copies of the second target nucleic acid molecule can then be subjected to DNA sequencing, preferably using a “Next-Generation” massively parallel DNA sequencing platform (e.g., FIG. 1B).

The sequence reads produced from either the first strand target nucleic acid molecule and the second strand target nucleic acid molecule derived from the original double-stranded target nucleic acid molecule can be identified based on sharing a related substantially unique SMI and distinguished from the opposite strand target nucleic acid molecule by virtue of an SDE. In some embodiments the SMI may be a sequence based on a mathematically-based error correction code (for example, a Hamming code), whereby certain amplification errors, sequencing errors or SMI synthesis errors can be tolerated for the purpose of relating the sequences of the SMI sequences on complementary strands of an original Duplex (e.g., a double-stranded nucleic acid molecule). For example, with a double stranded exogenous SMI where the SMI comprises 15 base pairs of fully degenerate sequence of canonical DNA bases, an estimated 4\hat{ }15=1,073,741,824 SMI variants will exist in a population of the fully degenerate SMIs. If two SMIs are recovered from reads of sequencing data that differ by only one nucleotide within the SMI sequence out of a population of 10,000 sampled SMIs, it can be mathematically calculated the probability of this occurring by random chance and a decision made whether it is more probable that the single base pair difference reflects one of the aforementioned types of errors and the SMI sequences could be determined to have in fact derived from the same original duplex molecule. In some embodiments where the SMI is, at least in part, an exogenously applied sequence where the sequence variants are not fully degenerate to each other and are, at least in part, known sequences, the identity of the known sequences can in some embodiments be designed in such a way that one or more errors of the aforementioned types will not convert the identity of one known SMI sequence to that of another SMI sequence, such that the probability of one SMI being misinterpreted as that of another SMI is reduced. In some embodiments this SMI design strategy comprises a Hamming Code approach or derivative thereof. Once identified, one or more sequence reads produced from the first strand target nucleic acid molecule are compared with one or more sequence reads produced from the second strand target nucleic acid molecule to produce an error-corrected target nucleic acid molecule sequence (e.g., FIG. 1C). For example, nucleotide positions where the bases from both the first and second strand target nucleic acid sequences agree are deemed to be true sequences, whereas nucleotide positions that disagree between the two strands are recognized as potential sites of technical errors that may be discounted, eliminated, corrected or otherwise identified. An error-corrected sequence of the original double-stranded target nucleic acid molecule can thus be produced (shown in FIG. 1C). In some embodiments and following separately grouping of each of the sequencing reads produced from the first strand target nucleic acid molecule and the second strand target nucleic acid molecule, a single-strand consensus sequence can be generated for each of the first and second strands. The single-stranded consensus sequences from the first strand target nucleic acid molecule and the second strand target nucleic acid molecule can then be compared to produce an error-corrected target nucleic acid molecule sequence (e.g., FIG. 1C).

Alternatively, in some embodiments, sites of sequence disagreement between the two strands can be recognized as potential sites of biologically-derived mismatches in the original double stranded target nucleic acid molecule. Alternatively, in some embodiments, sites of sequence disagreement between the two strands can be recognized as potential sites of DNA synthesis-derived mismatches in the original double stranded target nucleic acid molecule. Alternatively, in some embodiments, sites of sequence disagreement between the two strands can be recognized as potential sites where a damaged or modified nucleotide base was present on one or both strands and was converted to a mismatch by an enzymatic process (for example a DNA polymerase, a DNA glycosylase or another nucleic acid modifying enzyme or chemical process). In some embodiments, this latter finding can be used to infer the presence of nucleic acid damage or nucleotide modification prior to the enzymatic process or chemical treatment.

In some embodiments, and in accordance with aspects of the present technology, sequencing reads generated from the Duplex Sequencing steps discussed herein can be further filtered to eliminate sequencing reads from DNA-damaged molecules (e.g., damaged during storage, shipping, during or following tissue or blood extraction, during or following library preparation, etc.). For example, DNA repair enzymes, such as Uracil-DNA Glycosylase (UDG), Formamidopyrimidine DNA glycosylase (FPG), and 8-oxoguanine DNA glycosylase (OGG1), can be utilized to eliminate or correct DNA damage (e.g., in vitro DNA damage or in vivo damage). These DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., a common DNA lesion that results from reactive oxygen species). FPG also has lyase activity that can generate a 1 base gap at abasic sites. Such abasic sites will generally subsequently fail to amplify by PCR, for example, because the polymerase fails to copy the template. Accordingly, the use of such DNA damage repair/elimination enzymes can effectively remove damaged DNA that doesn't have a true mutation but might otherwise be undetected as an error following sequencing and duplex sequence analysis. Although an error due to a damaged base can often be corrected by Duplex Sequencing in rare cases a complementary error could theoretically occur at the same position on both strands, thus, reducing error-increasing damage can reduce the probability of artifacts. Furthermore, during library preparation certain fragments of DNA to be sequenced may be single-stranded from their source or from processing steps (for example, mechanical DNA shearing). These regions are typically converted to double stranded DNA during an “end repair” step known in the art, whereby a DNA polymerase and nucleoside substrates are added to a DNA sample to extend 5′ recessed ends. A mutagenic site of DNA damage in the single-stranded portion of the DNA being copied (i.e. single-stranded 5′ overhang at one or both ends of the DNA duplex or internal single-stranded nicks or gaps) can cause an error during the fill-in reaction that could render a single-stranded mutation, synthesis error or site of nucleic acid damage into a double-stranded form that could be misinterpreted in the final duplex consensus sequence as a true mutation whereby the true mutation was present in the original double stranded nucleic acid molecule, when, in fact, it was not. This scenario, termed “pseudo-duplex”, can be reduced or prevented by use of such damage destroying/repair enzymes. In other embodiments this occurrence can be reduced or eliminated through use of strategies to destroy or prevent single-stranded portions of the original duplex molecule to form (e.g. use of certain enzymes being used to fragment the original double stranded nucleic acid material rather than mechanical shearing or certain other enzymes that may leave nicks or gaps). In other embodiments use of processes to eliminate single-stranded portions of original double-stranded nucleic acids (e.g. single-stand specific nucleases such as S1 nuclease or mung bean nuclease) can be utilized for a similar purpose.

In further embodiments, sequencing reads generated from the Duplex Sequencing steps discussed herein can be further filtered to eliminate false mutations by trimming ends of the reads most prone to pseudoduplex artifacts. For example, DNA fragmentation can generate single strand portions at the terminal ends of double-stranded molecule. These single-stranded portions can be filled in (e.g., by Klenow or T4 polymerase) during end repair. In some instances, polymerases make copy mistakes in these end repaired regions leading to the generation of “pseudoduplex molecules.” These artifacts of library preparation can incorrectly appear to be true mutations once sequenced. These errors, as a result of end repair mechanisms, can be eliminated or reduced from analysis post-sequencing by trimming the ends of the sequencing reads to exclude any mutations that may have occurred in higher risk regions, thereby reducing the number of false mutations. In one embodiment, such trimming of sequencing reads can be accomplished automatically (e.g., a normal process step). In another embodiment, a mutant frequency can be assessed for fragment end regions and if a threshold level of mutations is observed in the fragment end regions, sequencing read trimming can be performed before generating a double-strand consensus sequence read of the DNA fragments.

By way of specific example, in some embodiments, provided herein are methods of generating an error-corrected sequence read of a double-stranded target nucleic acid material, including the step of ligating a double-stranded target nucleic acid material to at least one adapter sequence, to form an adapter-target nucleic acid material complex, wherein the at least one adapter sequence comprises (a) a degenerate or semi-degenerate single molecule identifier (SMI) sequence that uniquely labels each molecule of the double-stranded target nucleic acid material, and (b) a first nucleotide adapter sequence that tags a first strand of the adapter-target nucleic acid material complex, and a second nucleotide adapter sequence that is at least partially non-complimentary to the first nucleotide sequence that tags a second strand of the adapter-target nucleic acid material complex such that each strand of the adapter-target nucleic acid material complex has a distinctly identifiable nucleotide sequence relative to its complementary strand. The method can next include the steps of amplifying each strand of the adapter-target nucleic acid material complex to produce a plurality of first strand adapter-target nucleic acid complex amplicons and a plurality of second strand adapter-target nucleic acid complex amplicons. The method can further include the steps of amplifying both the first and strands to provide a first nucleic acid product and a second nucleic acid product. The method may also include the steps of sequencing each of the first nucleic acid product and second nucleic acid product to produce a plurality of first strand sequence reads and plurality of second strand sequence reads, and confirming the presence of at least one first strand sequence read and at least one second strand sequence read. The method may further include comparing the at least one first strand sequence read with the at least one second strand sequence read, and generating an error-corrected sequence read of the double-stranded target nucleic acid material by discounting nucleotide positions that do not agree, or alternatively removing compared first and second strand sequence reads having one or more nucleotide positions where the compared first and second strand sequence reads are non-complementary.

By way of an additional specific example, in some embodiments, provided herein are methods of identifying a DNA variant from a sample including the steps of ligating both strands of a nucleic acid material (e.g., a double-stranded target DNA molecule) to at least one asymmetric adapter molecule to form an adapter-target nucleic acid material complex having a first nucleotide sequence associated with a first strand of a double-stranded target DNA molecule (e.g., a top strand) and a second nucleotide sequence that is at least partially non-complementary to the first nucleotide sequence associated with a second strand of the double-stranded target DNA molecule (e.g., a bottom strand), and amplifying each strand of the adapter-target nucleic acid material, resulting in each strand generating a distinct yet related set of amplified adapter-target nucleic acid products. The method can further include the steps of sequencing each of a plurality of first strand adapter-target nucleic acid products and a plurality of second strand adapter-target nucleic acid products, confirming the presence of at least one amplified sequence read from each strand of the adapter-target nucleic acid material complex, and comparing the at least one amplified sequence read obtained from the first strand with the at least one amplified sequence read obtained from the second strand to form a consensus sequence read of the nucleic acid material (e.g., a double-stranded target DNA molecule) having only nucleotide bases at which the sequence of both strands of the nucleic acid material (e.g., a double-stranded target DNA molecule) are in agreement, such that a variant occurring at a particular position in the consensus sequence read (e.g., as compared to a reference sequence) is identified as a true DNA variant.

In some embodiments, provided herein are methods of generating a high accuracy consensus sequence from a double-stranded nucleic acid material, including the steps of tagging individual duplex DNA molecules with an adapter molecule to form tagged DNA material, wherein each adapter molecule comprises (a) a degenerate or semi-degenerate single molecule identifier (SMI) that uniquely labels the duplex DNA molecule, and (b) first and second non-complementary nucleotide adapter sequences that distinguishes an original top strand from an original bottom strand of each individual DNA molecule within the tagged DNA material, for each tagged DNA molecule, and generating a set of duplicates of the original top strand of the tagged DNA molecule and a set of duplicates of the original bottom strand of the tagged DNA molecule to form amplified DNA material. The method can further include the steps of creating a first single strand consensus sequence (SSCS) from the duplicates of the original top strand and a second single strand consensus sequence (SSCS) from the duplicates of the original bottom strand, comparing the first SSCS of the original top strand to the second SSCS of the original bottom strand, and generating a high-accuracy consensus sequence having only nucleotide bases at which the sequence of both the first SSCS of the original top strand and the second SSCS of the original bottom strand are complimentary.

In further embodiments, provided herein are methods of detecting and/or quantifying DNA damage from a sample comprising double-stranded target DNA molecules including the steps of ligating both strands of each double-stranded target DNA molecule to at least one asymmetric adapter molecule to form a plurality of adapter-target DNA complexes, wherein each adapter-target DNA complex has a first nucleotide sequence associated with a first strand of a double-stranded target DNA molecule and a second nucleotide sequence that is at least partially non-complementary to the first nucleotide sequence associated with a second strand of the double-stranded target DNA molecule, and for each adapter target DNA complex: amplifying each strand of the adapter-target DNA complex, resulting in each strand generating a distinct yet related set of amplified adapter-target DNA amplicons. The method can further include the steps of sequencing each of a plurality of first strand adapter-target DNA amplicons and a plurality of second strand adapter-target DNA amplicons, confirming the presence of at least one sequence read from each strand of the adapter-target DNA complex, and comparing the at least one sequence read obtained from the first strand with the at least one sequence read obtained from the second strand to detect and/or quantify nucleotide bases at which the sequence read of one strand of the double-stranded DNA molecule is in disagreement (e.g., non-complimentary) with the sequence read of the other strand of the double-stranded DNA molecule, such that site(s) of DNA damage can be detected and/or quantified. In some embodiments, the method can further include the steps of creating a first single strand consensus sequence (SSCS) from the first strand adapter-target DNA amplicons and a second single strand consensus sequence (SSCS) from the second strand adapter-target DNA amplicons, comparing the first SSCS of the original first strand to the second SSCS of the original second strand, and identifying nucleotide bases at which the sequence of the first SSCS and the second SSCS are non-complementary to detect and/or quantify DNA damage associated with the double-stranded target DNA molecules in the sample.

Single Molecule Identifier Sequences (SMIs)

In accordance with various embodiments, provided methods and compositions include one or more SMI sequences on each strand of a nucleic acid material. The SMI can be independently carried by each of the single strands that result from a double-stranded nucleic acid molecule such that the derivative amplification products of each strand can be recognized as having come from the same original substantially unique double-stranded nucleic acid molecule after sequencing. In some embodiments, the SMI may include additional information and/or may be used in other methods for which such molecule distinguishing functionality is useful, as will be recognized by one of skill in the art. In some embodiments, an SMI element may be incorporated before, substantially simultaneously, or after adapter sequence ligation to a nucleic acid material.

In some embodiments, an SMI sequence may include at least one degenerate or semi-degenerate nucleic acid. In other embodiments, an SMI sequence may be non-degenerate. In some embodiments, the SMI can be the sequence associated with or near a fragment end of the nucleic acid molecule (e.g., randomly or semi-randomly sheared ends of ligated nucleic acid material). In some embodiments, an exogenous sequence may be considered in conjunction with the sequence corresponding to randomly or semi-randomly sheared ends of ligated nucleic acid material (e.g., DNA) to obtain an SMI sequence capable of distinguishing, for example, single DNA molecules from one another. In some embodiments, a SMI sequence is a portion of an adapter sequence that is ligated to a double-strand nucleic acid molecule. In certain embodiments, the adapter sequence comprising a SMI sequence is double-stranded such that each strand of the double-stranded nucleic acid molecule includes an SMI following ligation to the adapter sequence. In another embodiment, the SMI sequence is single-stranded before or after ligation to a double-stranded nucleic acid molecule and a complimentary SMI sequence can be generated by extending the opposite strand with a DNA polymerase to yield a complementary double-stranded SMI sequence. In other embodiments, an SMI sequence is in a single-stranded portion of the adapter (e.g., an arm of an adapter having a Y-shape). In such embodiments, the SMI can facilitate grouping of families of sequence reads derived from an original strand of a double-stranded nucleic acid molecule, and in some instances can confer relationship between original first and second strands of a double-stranded nucleic acid molecule (e.g., all or part of the SMIs maybe relatable via look up table). In embodiments, where the first and second strands are labeled with different SMIs, the sequence reads from the two original strands may be related using one or more of an endogenous SMI (e.g., a fragment-specific feature such as sequence associated with or near a fragment end of the nucleic acid molecule), or with use of an additional molecular tag shared by the two original strands (e.g., a barcode in a double-stranded portion of the adapter, or a combination thereof. In some embodiments, each SMI sequence may include between about 1 to about 30 nucleic acids (e.g., 1, 2, 3, 4, 5, 8, 10, 12, 14, 16, 18, 20, or more degenerate or semi-degenerate nucleic acids).

In some embodiments, a SMI is capable of being ligated to one or both of a nucleic acid material and an adapter sequence. In some embodiments, a SMI may be ligated to at least one of a T-overhang, an A-overhang, a CG-overhang, a dehydroxylated base, and a blunt end of a nucleic acid material.

In some embodiments, a sequence of a SMI may be considered in conjunction with (or designed in accordance with) the sequence corresponding to, for example, randomly or semi-randomly sheared ends of a nucleic acid material (e.g., a ligated nucleic acid material), to obtain a SMI sequence capable of distinguishing single nucleic acid molecules from one another.

In some embodiments, at least one SMI may be an endogenous SMI (e.g., an SMI related to a shear point (e.g., a fragment end), for example, using the shear point itself or using a defined number of nucleotides in the nucleic acid material immediately adjacent to the shear point [e.g., 2, 3, 4, 5, 6, 7, 8, 9, 10 nucleotides from the shear point]). In some embodiments, at least one SMI may be an exogenous SMI (e.g., an SMI comprising a sequence that is not found on a target nucleic acid material).

In some embodiments, a SMI may be or comprise an imaging moiety (e.g., a fluorescent or otherwise optically detectable moiety). In some embodiments, such SMIs allow for detection and/or quantitation without the need for an amplification step.

In some embodiments a SMI element may comprise two or more distinct SMI elements that are located at different locations on the adapter-target nucleic acid complex.

Various embodiments of SMIs are further disclosed in International Patent Publication No. WO2017/100441, which is incorporated by reference herein in its entirety.

Strand-Defining Element (SDE)

In some embodiments, each strand of a double-stranded nucleic acid material may further include an element that renders the amplification products of the two single-stranded nucleic acids that form the target double-stranded nucleic acid material substantially distinguishable from each other after sequencing. In some embodiments, a SDE may be or comprise asymmetric primer sites comprised within a sequencing adapter, or, in other arrangements, sequence asymmetries may be introduced into the adapter sequences and not within the primer sequences, such that at least one position in the nucleotide sequences of a first strand target nucleic acid sequence complex and a second stand of the target nucleic acid sequence complex are different from each other following amplification and sequencing. In other embodiments, the SDE may comprise another biochemical asymmetry between the two strands that differs from the canonical nucleotide sequences A, T, C, G or U, but is converted into at least one canonical nucleotide sequence difference in the two amplified and sequenced molecules. In yet another embodiment, the SDE may be or comprise a means of physically separating the two strands before amplification, such that derivative amplification products from the first strand target nucleic acid sequence and the second strand target nucleic acid sequence are maintained in substantial physical isolation from one another for the purposes of maintaining a distinction between the two derivative amplification products. Other such arrangements or methodologies for providing an SDE function that allows for distinguishing the first and second strands may be utilized.

In some embodiments, a SDE may be capable of forming a loop (e.g., a hairpin loop). In some embodiments, a loop may comprise at least one endonuclease recognition site. In some embodiments the target nucleic acid complex may contain an endonuclease recognition site that facilitates a cleavage event within the loop. In some embodiments a loop may comprise a non-canonical nucleotide sequence. In some embodiments the contained non-canonical nucleotide may be recognizable by one or more enzyme that facilitates strand cleavage. In some embodiments the contained non-canonical nucleotide may be targeted by one or more chemical process facilitates strand cleavage in the loop. In some embodiments the loop may contain a modified nucleic acid linker that may be targeted by one or more enzymatic, chemical or physical process that facilitates strand cleavage in the loop. In some embodiments this modified linker is a photocleavable linker.

A variety of other molecular tools could serve as SMIs and SDEs. Other than shear points and DNA-based tags, single-molecule compartmentalization methods that keep paired strands in physical proximity or other non-nucleic acid tagging methods could serve the strand-relating function. Similarly, asymmetric chemical labelling of the adapter strands in a way that they can be physically separated can serve an SDE role. A recently described variation of Duplex Sequencing uses bisulfite conversion to transform naturally occurring strand asymmetries in the form of cytosine methylation into sequence differences that distinguish the two strands. Although this implementation limits the types of mutations that can be detected, the concept of capitalizing on native asymmetry is noteworthy in the context of emerging sequencing technologies that can directly detect modified nucleotides. Various embodiments of SDEs are further disclosed in International Patent Publication No. WO2017/100441, which is incorporated by reference in its entirety.

Adapters and Adapter Sequences

In various arrangements, adapter molecules that comprise SMIs (e.g., molecular barcodes), SDEs, primer sites, flow cell sequences and/or other features are contemplated for use with many of the embodiments disclosed herein. In some embodiments, provided adapters may be or comprise one or more sequences complimentary or at least partially complimentary to PCR primers (e.g., primer sites) that have at least one of the following properties: 1) high target specificity; 2) capable of being multiplexed; and 3) exhibit robust and minimally biased amplification.

In some embodiments, adapter molecules can be “Y”-shaped, “U”-shaped, “hairpin” shaped, have a bubble (e.g., a portion of sequence that is non-complimentary), or other features. In other embodiments, adapter molecules can comprise a “Y”-shape, a “U”-shaped, a “hairpin” shaped, or a bubble. Certain adapters may comprise modified or non-standard nucleotides, restriction sites, or other features for manipulation of structure or function in vitro. Adapter molecules may ligate to a variety of nucleic acid material having a terminal end. For example, adapter molecules can be suited to ligate to a T-overhang, an A-overhang, a CG-overhang, a multiple nucleotide overhang, a dehydroxylated base, a blunt end of a nucleic acid material and the end of a molecule were the 5′ of the target is dephosphorylated or otherwise blocked from traditional ligation. In other embodiments the adapter molecule can contain a dephosphorylated or otherwise ligation-preventing modification on the 5′ strand at the ligation site. In the latter two embodiments such strategies may be useful for preventing dimerization of library fragments or adapter molecules.

An adapter sequence can mean a single-strand sequence, a double-strand sequence, a complimentary sequence, a non-complimentary sequence, a partial complimentary sequence, an asymmetric sequence, a primer binding sequence, a flow-cell sequence, a ligation sequence or other sequence provided by an adapter molecule. In particular embodiments, an adapter sequence can mean a sequence used for amplification by way of compliment to an oligonucleotide.

In some embodiments, provided methods and compositions include at least one adapter sequence (e.g., two adapter sequences, one on each of the 5′ and 3′ ends of a nucleic acid material). In some embodiments, provided methods and compositions may comprise 2 or more adapter sequences (e.g., 3, 4, 5, 6, 7, 8, 9, 10 or more). In some embodiments, at least two of the adapter sequences differ from one another (e.g., by sequence). In some embodiments, each adapter sequence differs from each other adapter sequence (e.g., by sequence). In some embodiments, at least one adapter sequence is at least partially non-complementary to at least a portion of at least one other adapter sequence (e.g., is non-complementary by at least one nucleotide).

In some embodiments, an adapter sequence comprises at least one non-standard nucleotide. In some embodiments, a non-standard nucleotide is selected from an abasic site, a uracil, tetrahydrofuran, 8-oxo-7,8-dihydro-2′deoxyadenosine (8-oxo-A), 8-oxo-7,8-dihydro-2′-deoxyguanosine (8-oxo-G), deoxyinosine, 5′nitroindole, 5-Hydroxymethyl-2′-deoxycytidine, iso-cytosine, 5′-methyl-isocytosine, or isoguanosine, a methylated nucleotide, an RNA nucleotide, a ribose nucleotide, an 8-oxo-guanine, a photocleavable linker, a biotinylated nucleotide, a desthiobiotin nucleotide, a thiol modified nucleotide, an acrydite modified nucleotide an iso-dC, an iso dG, a 2′-O-methyl nucleotide, an inosine nucleotide Locked Nucleic Acid, a peptide nucleic acid, a 5 methyl dC, a 5-bromo deoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, an 5′ Hexynyl modified nucleotide, an 5-Octadiynyl dU, photocleavable spacer, a non-photocleavable spacer, a click chemistry compatible modified nucleotide, and any combination thereof.

In some embodiments, an adapter sequence comprises a moiety having a magnetic property (i.e., a magnetic moiety). In some embodiments this magnetic property is paramagnetic. In some embodiments where an adapter sequence comprises a magnetic moiety (e.g., a nucleic acid material ligated to an adapter sequence comprising a magnetic moiety), when a magnetic field is applied, an adapter sequence comprising a magnetic moiety is substantially separated from adapter sequences that do not comprise a magnetic moiety (e.g., a nucleic acid material ligated to an adapter sequence that does not comprise a magnetic moiety).

In some embodiments, at least one adapter sequence is located 5′ to a SMI. In some embodiments, at least one adapter sequence is located 3′ to a SMI.

In some embodiments, an adapter sequence may be linked to at least one of a SMI and a nucleic acid material via one or more linker domains. In some embodiments, a linker domain may be comprised of nucleotides. In some embodiments, a linker domain may include at least one modified nucleotide or non-nucleotide molecules (for example, as described elsewhere in this disclosure). In some embodiments, a linker domain may be or comprise a loop.

In some embodiments, an adapter sequence on either or both ends of each strand of a double-stranded nucleic acid material may further include one or more elements that provide a SDE. In some embodiments, a SDE may be or comprise asymmetric primer sites comprised within the adapter sequences.

In some embodiments, an adapter sequence may be or comprise at least one SDE and at least one ligation domain (i.e., a domain amendable to the activity of at least one ligase, for example, a domain suitable to ligating to a nucleic acid material through the activity of a ligase). In some embodiments, from 5′ to 3′, an adapter sequence may be or comprise a primer binding site, a SDE, and a ligation domain.

Various methods for synthesizing Duplex Sequencing adapters have been previously described in, e.g., U.S. Pat. No. 9,752,188, International Patent Publication No. WO2017/100441, and International Patent Application No. PCT/US18/59908 (filed Nov. 8, 2018), all of which are incorporated by reference herein in their entireties.

Primers

In some embodiments, one or more PCR primers that have at least one of the following properties: 1) high target specificity; 2) capable of being multiplexed; and 3) exhibit robust and minimally biased amplification are contemplated for use in various embodiments in accordance with aspects of the present technology. A number of prior studies and commercial products have designed primer mixtures satisfying certain of these criteria for conventional PCR-CE. However, it has been noted that these primer mixtures are not always optimal for use with MPS. Indeed, developing highly multiplexed primer mixtures can be a challenging and time-consuming process. Conveniently, both Illumina and Promega have recently developed multiplex compatible primer mixtures for the Illumina platform that show robust and efficient amplification of a variety of standard and non-standard STR and SNP loci. Because these kits use PCR to amplify their target regions prior to sequencing, the 5′-end of each read in paired-end sequencing data corresponds to the 5′-end of the PCR primers used to amplify the DNA. In some embodiments, provided methods and compositions include primers designed to ensure uniform amplification, which may entail varying reaction concentrations, melting temperatures, and minimizing secondary structure and intra/inter-primer interactions. Many techniques have been described for highly multiplexed primer optimization for MPS applications. In particular, these techniques are often known as ampliseq methods, as well described in the art.

Amplification

Provided methods and compositions, in various embodiments, make use of, or are of use in, at least one amplification step wherein a nucleic acid material (or portion thereof, for example, a specific target region or locus) is amplified to form an amplified nucleic acid material (e.g., some number of amplicon products).

In some embodiments, amplifying a nucleic acid material includes a step of amplifying nucleic acid material derived from each of a first and second nucleic acid strand from an original double-stranded nucleic acid material using at least one single-stranded oligonucleotide at least partially complementary to a sequence present in a first adapter sequence such that a SMI sequence is at least partially maintained. An amplification step further includes employing a second single-stranded oligonucleotide to amplify each strand of interest, and such second single-stranded oligonucleotide can be (a) at least partially complementary to a target sequence of interest, or (b) at least partially complementary to a sequence present in a second adapter sequence such that the at least one single-stranded oligonucleotide and a second single-stranded oligonucleotide are oriented in a manner to effectively amplify the nucleic acid material.

In some embodiments, amplifying nucleic acid material in a sample can include amplifying nucleic acid material in “tubes” (e.g., PCR tubes), in emulsion droplets, microchambers, and other examples described above or other known vessels.

In some embodiments, at least one amplifying step includes at least one primer that is or comprises at least one non-standard nucleotide. In some embodiments, a non-standard nucleotide is selected from a uracil, a methylated nucleotide, an RNA nucleotide, a ribose nucleotide, an 8-oxo-guanine, a biotinylated nucleotide, a locked nucleic acid, a peptide nucleic acid, a high-Tm nucleic acid variant, an allele discriminating nucleic acid variant, any other nucleotide or linker variant described elsewhere herein and any combination thereof.

While any application-appropriate amplification reaction is contemplated as compatible with some embodiments, by way of specific example, in some embodiments, an amplification step may be or comprise a polymerase chain reaction (PCR), rolling circle amplification (RCA), multiple displacement amplification (MDA), isothermal amplification, polony amplification within an emulsion, bridge amplification on a surface, the surface of a bead or within a hydrogel, and any combination thereof.

In some embodiments, amplifying a nucleic acid material includes use of single-stranded oligonucleotides at least partially complementary to regions of the adapter sequences on the 5′ and 3′ ends of each strand of the nucleic acid material. In some embodiments, amplifying a nucleic acid material includes use of at least one single-stranded oligonucleotide at least partially complementary to a target region or a target sequence of interest (e.g., a genomic sequence, a mitochondrial sequence, a plasmid sequence, a synthetically produced target nucleic acid, etc.) and a single-stranded oligonucleotide at least partially complementary to a region of the adapter sequence (e.g., a primer site).

In general, robust amplification, for example PCR amplification, can be highly dependent on the reaction conditions. Multiplex PCR, for example, can be sensitive to buffer composition, monovalent or divalent cation concentration, detergent concentration, crowding agent (i.e. PEG, glycerol, etc.) concentration, primer concentrations, primer Tms, primer designs, primer GC content, primer modified nucleotide properties, and cycling conditions (i.e. temperature and extension times and rate of temperature changes). Optimization of buffer conditions can be a difficult and time-consuming process. In some embodiments, an amplification reaction may use at least one of a buffer, primer pool concentration, and PCR conditions in accordance with a previously known amplification protocol. In some embodiments, a new amplification protocol may be created, and/or an amplification reaction optimization may be used. By way of specific example, in some embodiments, a PCR optimization kit may be used, such as a PCR Optimization Kit from Promega®, which contains a number of pre-formulated buffers that are partially optimized for a variety of PCR applications, such as multiplex, real-time, GC-rich, and inhibitor-resistant amplifications. These pre-formulated buffers can be rapidly supplemented with different Mg²⁺ and primer concentrations, as well as primer pool ratios. In addition, in some embodiments, a variety of cycling conditions (e.g., thermal cycling) may be assessed and/or used. In assessing whether or not a particular embodiment is appropriate for a particular desired application, one or more of specificity, allele coverage ratio for heterozygous loci, interlocus balance, and depth, among other aspects may be assessed. Measurements of amplification success may include DNA sequencing of the products, evaluation of products by gel or capillary electrophoresis or HPLC or other size separation methods followed by fragment visualization, melt curve analysis using double-stranded nucleic acid binding dyes or fluorescent probes, mass spectrometry or other methods known in the art.

In accordance with various embodiments, any of a variety of factors may influence the length of a particular amplification step (e.g., the number of cycles in a PCR reaction, etc.). For example, in some embodiments, a provided nucleic acid material may be compromised or otherwise suboptimal (e.g. degraded and/or contaminated). In such case, a longer amplification step may be helpful in ensuring a desired product is amplified to an acceptable degree. In some embodiments an amplification step may provide an average of 3 to 10 sequenced PCR copies from each starting DNA molecule, though in other embodiments, only a single copy of each of a first strand and second strand are required. Without wishing to be held to a particular theory, it is possible that too many or too few PCR copies could result in reduced assay efficiency and, ultimately, reduced depth. Generally, the number of nucleic acid (e.g., DNA) fragments used in an amplification (e.g., PCR) reaction is a primary adjustable variable that can dictate the number of reads that share the same SMI/barcode sequence.

### Nucleic Acid Material

Types

In accordance with various embodiments, any of a variety of nucleic acid material may be used. In some embodiments, nucleic acid material may comprise at least one modification to a polynucleotide within the canonical sugar-phosphate backbone. In some embodiments, nucleic acid material may comprise at least one modification within any base in the nucleic acid material. For example, by way of non-limiting example, in some embodiments, the nucleic acid material is or comprises at least one of double-stranded DNA, single-stranded DNA, double-stranded RNA, single-stranded RNA, peptide nucleic acids (PNAs), locked nucleic acids (LNAs).

Modifications

In accordance with various embodiments, nucleic acid material may receive one or more modifications prior to, substantially simultaneously, or subsequent to, any particular step, depending upon the application for which a particular provided method or composition is used.

In some embodiments, a modification may be or comprise repair of at least a portion of the nucleic acid material. While any application-appropriate manner of nucleic acid repair is contemplated as compatible with some embodiments, certain exemplary methods and compositions therefore are described below and in the Examples.

By way of non-limiting example, in some embodiments, DNA repair enzymes, such as Uracil-DNA Glycosylase (UDG), Formamidopyrimidine DNA glycosylase (FPG), and 8-oxoguanine DNA glycosylase (OGG1), can be utilized to correct DNA damage (e.g., in vitro DNA damage). As discussed above, these DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., most common DNA lesion that results from reactive oxygen species). FPG also has lyase activity that can generate 1 base gap at abasic sites. Such abasic sites will subsequently fail to amplify by PCR, for example, because the polymerase fails copy the template. Accordingly, the use of such DNA damage repair enzymes can effectively remove damaged DNA that doesn't have a true mutation, but might otherwise be undetected as an error following sequencing and duplex sequence analysis.

As discussed above, in further embodiments, sequencing reads generated from the processing steps discussed herein can be further filtered to eliminate false mutations by trimming ends of the reads most prone to artifacts. For example, DNA fragmentation can generate single-strand portions at the terminal ends of double-stranded molecules. These single-stranded portions can be filled in (e.g., by Klenow) during end repair. In some instances, polymerases make copy mistakes in these end-repaired regions leading to the generation of “pseudoduplex molecules.” These artifacts can appear to be true mutations once sequenced. These errors, as a result of end repair mechanisms, can be eliminated from analysis post-sequencing by trimming the ends of the sequencing reads to exclude any mutations that may have occurred, thereby reducing the number of false mutations. In some embodiments, such trimming of sequencing reads can be accomplished automatically (e.g., a normal process step). In some embodiments, a mutant frequency can be assessed for fragment end regions and if a threshold level of mutations is observed in the fragment end regions, sequencing read trimming can be performed before generating a double-strand consensus sequence read of the DNA fragments.

The high degree of error correction provided by the strand-comparison technology of Duplex Sequencing reduces sequencing errors of double-stranded nucleic acid molecules by multiple orders of magnitude as compared with standard next-generation sequencing methods. This reduction in errors improves the accuracy of sequencing in nearly all types of sequences but can be particularly well suited to biochemically challenging sequences that are well known in the art to be particularly error prone. One non-limiting example of such type of sequence is homopolymers or other microsatellites/short-tandem repeats. Another non-limiting example of error prone sequences that benefit from Duplex Sequencing error correction are molecules that have been damaged, for example, by heating, radiation, mechanical stress, or a variety of chemical exposures which creates chemical adducts that are error prone during copying by one or more nucleotide polymerases and also those that create single-stranded DNA at ends of molecules or as nicks and gaps. In further embodiments, Duplex Sequencing can also be used for the accurate detection of minority sequence variants among a population of double-stranded nucleic acid molecules. One non-limiting example of this application is detection of a small number of DNA molecules derived from a cancer, among a larger number of unmutated molecules from non-cancerous tissues within a subject. Another non-limiting application for rare variant detection by Duplex Sequencing is early detection of DNA damage resulting from genotoxin exposure. A further non-limiting application of Duplex Sequencing is for detection of mutations generated from either genotoxic or non-genotoxic carcinogens by looking at genetic clones that are emerging with driver mutations. A yet further non-limiting application for accurate detection of minority sequence variants is to generate a mutagenic signature associated with a genotoxin.

### Identification and Assessment of Genotoxicity

The present technology is directed to methods, systems, kits, etc. for assessing genotoxicity. In particular, some embodiments of the technology are directed to utilizing Duplex Sequencing for assessing a genotoxic potential of a compound (e.g., a chemical compound) or other agent in a biological source. For example, various embodiments of the present technology include performing Duplex Sequencing methods that allow direct measurement of agent-induced mutations in any genomic context of any organism, and without need for clonal selection. Further examples of the present technology are directed to methods for detecting and assessing in vivo genomic mutagenesis using Duplex Sequencing. Various aspects of the present technology have many applications in both pre-clinical and clinical drug safety testing as well as other industry-wide implications. For example, the present technology includes methods for detecting ultra-low frequency mutations that cause the onset of diseases/disorders years later, wherein the mutations occur as a direct result of exposure to at least one genotoxin (e g radiation, carcinogen) and/or as a result of endogenous sources, such as DNA polymerase errors, free radicals, and depurination. The detection can occur via testing a subject after a recent exposure to a genotoxin (e.g. within days of exposure) and using Duplex Sequencing to identify the ultra-low frequency mutations. In particular examples, the ultra-low frequency mutations detected can be compared to mutations known to cause a specific disease or disorder, including those diseases/disorders that typically manifest after many years post-exposure (e.g. lung cancer 20 years after exposure to an asbestos). The present technology thus provides an expedient method of identifying the presence of genotoxins and victims exposed to them in order to prevent future exposures, and to provide early medical treatment. The present technology can also be used in a variety of high throughput screening methods to identify unsafe consumer products, pharmaceuticals and other industrial/commercial/manufacturing byproducts that comprise genotoxins in order to remove them from the market or the environment.

In a particular embodiment, genotoxic effects such as deletions, breaks and/or rearrangements can lead to cancer or another genotoxic associated disease to disorder if the damage does not immediately lead to cell death. For example, the nucleic acid damage may be sufficient enough for the subject to develop a genotoxic associated disease or disorder, and/or it may contribute to the activation or progression of another type of disease or disorder already existing in an exposed subject. Regions sensitive to breakage, called fragile sites, may result from genotoxic agents (e.g., chemicals, such as pesticides or certain chemotherapy drugs). Some chemicals have the ability to induce fragile sites in regions of the chromosome where oncogenes are present, which could lead to carcinogenic effects. Furthermore, occupational exposure to some mixtures of pesticides, manufacturing compounds or other hazardous materials are positively correlated with increased genotoxic damage in the exposed individuals. Investigation of genotoxicity potential, for example, prior to human exposure, is highly desirable for any potential genotoxin, such as a potential drug, cosmetic, consumer product, industrial/manufacture produce or by-product or other chemical compound under development. Likewise, in embodiments where exposure to a genotoxin is suspected, if the genotoxin(s) can be identified, then the subject can receive targeted therapeutic treatments, and/or the genotoxin can be removed to prevent future exposure to the subject and to others.

The ability to detect genotoxic effects of a potential genotoxic agent or factor and to quantify a potentially resultant mutagenic process in a manner that is both time and cost efficient, is both commercially and medically important. In a particular example, the ability to detect and quantify mutagenic processes of a potential genotoxin can be important for assessing cancer risk, identifying carcinogens and predicting the impact of exposure in humans. However, current tools are slow, cumbersome and/or limited in the information that they provide. As described above, in vivo testing and mammalian reporter systems, such as the BigBlue® mouse and rat, are currently utilized under Food and Drug Administration (FDA) regulations as a valid genotoxicity metric for determining the potential of compounds to cause DNA damage.

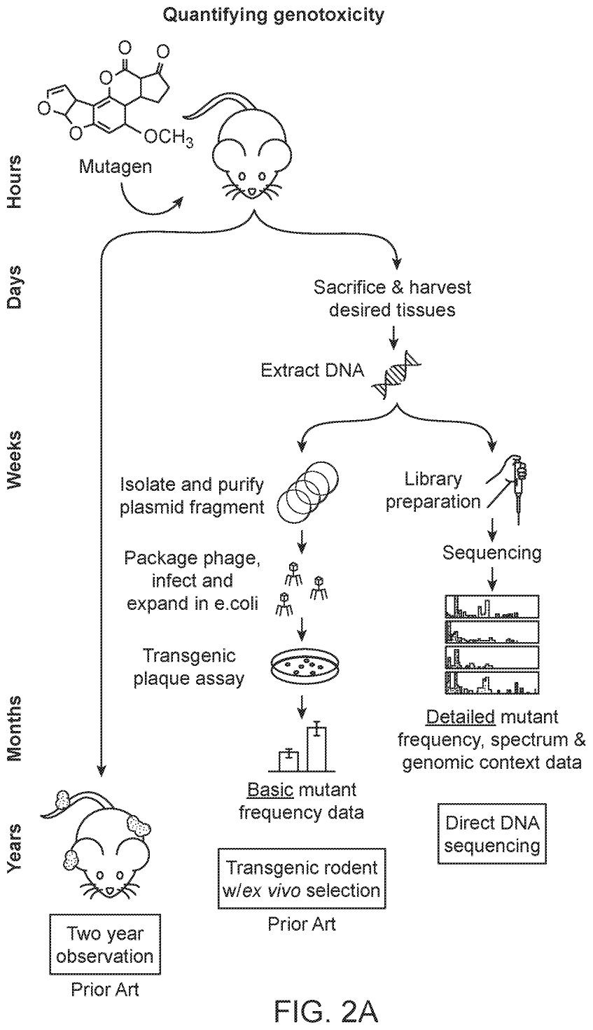

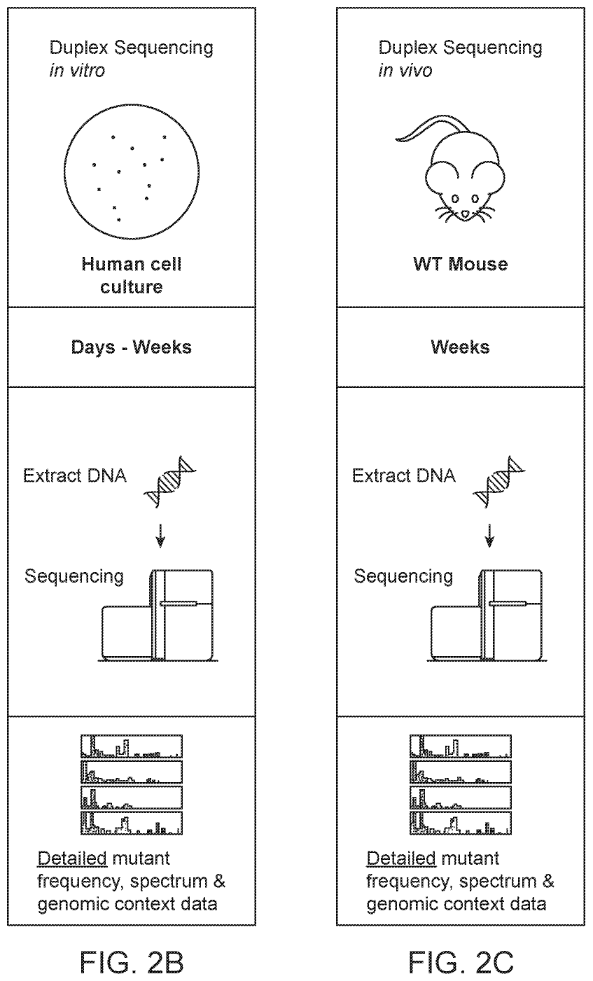

FIG. 2A is conceptual illustration showing various methodologies for assessing in vivo mutagenesis of a potential genotoxin (e.g., a potential mutagen). In each of the schemes illustrated in FIG. 2A, a test subject (e.g., BigBlue® mouse, a mouse model organism, a rat model organism, etc.) is exposed to the potential genotoxin (e.g., the compound/agent/factor under investigation) using an appropriate route of administration. In one conventional scheme shown on the far left-hand side of FIG. 2A, a long-term rodent carcinogenicity bioassay observes the test animal for a long period (e.g., 2 years) for the development of neoplastic lesions during or after exposure to various doses of the test substance. The test animals can be dosed by oral, dermal, or inhalation exposures, based upon the expected type of human exposure, for example. In conventional scheme, dosing typically lasts around two years; however dosing parameters (e.g., dosing duration, route of administration, dosing levels, or other dosing regimen parameters) can be set according to a desired test protocol. Referring to FIG. 2A, left-hand scheme, certain animal health features are monitored throughout the study, but the key assessment resides in the full pathological analysis of the test animals' tissues and organs when the study is terminated.

Another in vivo assay shown in the middle scheme of FIG. 2A, utilizes a transgenic rodent. Following an appropriate short-term dosing regimen (e.g., on the order of days or weeks), the test animal is sacrificed, desired tissues are harvested, and DNA is extracted. From the extracted DNA, the transgenic fragments are isolated and resultant purified plasmids are phage packaged and infected into E. coli. A conventional transgenic plaque assay is carried out and a basic mutant frequency is calculated.

Both of the above-described schemes are slow and provide very limited information with regard to genotoxicity (e.g., mutagenesis) of the tested potential genotoxin. The possibility of directly measuring somatic mutations in a way that is not restricted by genomic locus, tissue or organism is appealing, yet is currently impossible with standard DNA sequencing because of an error rate (˜10⁻³) well above the mutant frequency of normal tissues (˜10⁻⁷ to 10⁻⁸).

Massively parallel sequencing offers the possibility of comprehensively surveying the genome of any organism for the in vivo effect of mutagenic exposures, however, as discussed, conventional methods are far too inaccurate to detect such mutations, which may occur at a level of below one-in-a-million. For example, the error-rate of next-generation sequencing (NGS) at the approximately 0.1% creates a background noise that obscures the detection of rare variants and unique molecular profiles or signatures. Some common sources of errors in the NGS platforms include PCR enzymes (arising during amplification), sequencer reads, and DNA damage during processing (e.g., 8-oxo-guanine, deaminated cytosine, abasic sites and others).

In accordance with aspects of the present technology, Duplex Sequencing method steps can generate high-accuracy DNA sequencing reads that can further provide detailed mutant frequency (e.g., resolving genotoxin-induced mutations below one-in-a-million and provide a mutation spectrum data to objectively characterize different mutagenic processes and infer mechanism of action). For example, the right-hand scheme shown in FIG. 2A includes a method for quickly detecting and assessing genotoxicity of a potential genotoxin (e.g., potential mutagen) in the same test subject as the prior art schemes, while also providing detailed information about mutant frequency, spectrum of mutation type(s) and genomic context data. Moreover, Duplex Sequencing analysis can provide sensitive detection of mutagenesis at any genetic locus in any tissue from any organism. For example, and as illustrated in FIGS. 2A and 2B, Duplex Sequencing method schemes can be used for assessing in vitro mutagenesis of a test compound in cells (e.g., human cells, rodent cells, mammalian cells, non-mammalian cells, etc.) grown in culture (FIG. 2B) and for assessing in vivo mutagenesis of a test compound in a wild type rodent (e.g., mouse) (FIG. 2C). For example, in one embodiment, the present technology includes method steps including exposing a test organism (e.g., a rodent, cells grown in culture) to a test compound (e.g., potential genotoxin/mutagen) by an appropriate route of administration (e.g. orally, subcutaneous, topical, aerosol, intramuscular, etc.). In one embodiment, the test organism can be exposed to the test compound for a short duration (e.g., a single dose, a few minutes, a few hours, less than 24 hours, a few days, 2-6 days, etc.), or a moderate duration (e.g., several days, 3-12 days, approximately 1 week, approximately 2 weeks, approximately 1 month, approximately 2 months, approximately 3-6 months, etc.) or some other suitable amount of time. If the test organism is an animal (e.g., rodent), such as illustrated in FIG. 1A (right-hand scheme) and FIG. 1C, the animal may then be sacrificed and/or desired tissues harvested for DNA extraction. For example, in certain embodiments, the test animal is not sacrificed and one or more blood samples (e.g., at the same or different time points following administration or exposure to a test substance) can be collected from the test animal for DNA extraction. In embodiments where the animal is sacrificed, one or more tissues of interest (e.g., liver, bone marrow, lung, spleen, blood, etc.) can be harvested for DNA extraction. If the test organism comprises cells in culture (FIG. 1B), all or a portion of the cells can be collected for DNA extraction.

Following DNA extraction from the collected or harvested biological sample, a DNA library (e.g., a sequencing library) may be prepared. In one embodiment, an approach to prepare a DNA library (or other nucleic acid sequencing library) can begin with labelling (e.g., tagging) fragmented double-stranded nucleic acid material (e.g., from the DNA sample) with molecular barcodes in a similar manner as described above and with respect to a Duplex Sequencing library construction protocol (e.g., as illustrated in FIG. 1A). In some embodiments, the double-stranded nucleic acid material may be fragmented (e.g., such as with cell free DNA, damaged DNA, etc.); however, in other embodiments, various steps can include fragmentation of the nucleic acid material using mechanical shearing such as sonication, or other DNA cutting methods (e.g., enzymatic digestion, nebulization, etc.). Aspects of labelling the fragmented double-stranded nucleic acid material can include end-repair and 3′-dA-tailing, if required in a particular application, followed by ligation of the double-stranded nucleic acid fragments with Duplex Sequencing suitable adapters containing an SMI (e.g., as illustrated in FIG. 1A). In other embodiments, the SMI can be endogenous or a combination of exogenous and endogenous sequence for uniquely relating information from both strands of an original nucleic acid molecule.

Following ligation of adapter molecules to the double-stranded nucleic acid material, the method can continue with amplification (e.g., PCR amplification, rolling circle amplification, multiple displacement amplification, isothermal amplification, bridge amplification, surface bound amplification, etc.) (FIG. 1B). In certain embodiments, primers specific to, for example, one or more adapter sequences, can be used to amplify each strand of the nucleic acid material resulting in multiple copies of nucleic acid amplicons derived from each strand of an original double strand nucleic acid molecule, with each amplicon retaining the originally associated SMI (FIG. 1B). After amplification and associated steps to remove reaction byproducts, target nucleic acid region(s) (e.g., regions of interest, loci, etc.) can be optionally enriched using hybridization-based targeted capture, or in another embodiment, with multiplex PCR using primer(s) specific for an adapter sequence and primer(s) specific to the target nucleic acid region(s) of interest (not shown).

Following DNA library preparation and amplification steps, double-stranded adapter-DNA complexes can be sequenced with an appropriate massively parallel DNA sequencing platform using standard sequencing methods (FIG. 1B). Following sequencing of the multiple copies of the first strand the multiple copies of the second strand, sequencing data can be analyzed using a Duplex Sequencing approach and as described herein, whereby sequencing reads sharing the same exogenous (e.g., adapter sequence) and/or endogenous SMI that are derived from the first or second strand of the original double stranded target nucleic acid molecule are separately grouped. In some embodiments, the grouped sequencing reads from the first strand (e.g., “top strand”) are used to form a first strand consensus sequence (e.g., a single-strand consensus sequence (SSCS)) and the grouped sequencing reads from the second strand (e.g., “bottom strand”) are used to form a second strand consensus sequence (e.g., SSCS). Referring back to FIG. 1C, the first and second SSCSs can then be compared to generate a duplex consensus sequence (DCS) having nucleotides that are in agreement between the two strands (e.g., variants or mutations are considered to be true if they appear ill sequencing reads derived from both strands)(see, e.g., FIG. 1C). Likewise, in the comparing step, positions of the DCS where the nucleotides are not in agreement between the two strands can be further evaluated as potential sites of DNA damage, such as damage caused by the genotoxin exposure.

Referring back to FIGS. 2A-2C, and in accordance with aspects of the present technology, Duplex Sequencing analysis can further be used to precisely quantify the frequency of induced mutations across the genome. For example, aspects of the present technology are directed to generating genotoxicity-associated information captured in the derivative sequence data including, for example, mutation spectrum, trinucleotide mutational signatures, information about the functional consequences of certain mutations on proliferation and neoplastic selection, comparison to empirically-derived genotoxicity-associated information relating to known genotoxins (e.g., mutation spectra, trinucleotide mutational signatures), and the like.

The present technology further comprises a method for detecting at least one genomic mutation in a subject as a result of exposure to a genotoxin, comprising the steps of: 1) providing a sample from a subject following the genotoxin exposure, wherein the sample comprises a plurality of double-stranded DNA molecules; 2) ligating asymmetric adapter molecules to individual double-stranded DNA molecules to generate a plurality of adapter-DNA molecules; 3) for each adapter-DNA molecule: (i) generating a set of copies of an original first strand of the adapter-DNA molecule and a set of copies of an original second strand of the adapter-DNA molecule; (ii) sequencing the set of copies of the original first and second strands to provide a first strand sequence and a second strand sequence; and (iii) comparing the first strand sequence and the second strand sequence to identify one or more correspondences between the first and second strand sequences; and 4) analyzing the one or more correspondences in each of the adapter-DNA molecules to determine at least one of a mutant frequency and a mutation spectrum indicative of a specific genotoxin, a class of genotoxin, and/or a mechanism of action. In some embodiments, the mutation spectrum is a triplet mutation spectrum. In other embodiments, analyzing the one or more correspondences in each of the adapter-DNA molecules to determine a triplet mutation spectrum further comprises generating a triplet mutation signature for the specific genotoxin. In certain embodiments, determining a mutant frequency comprises determining a frequency of a triplet/trinucleotide context of the base that is mutated.

In some embodiments, the triplet mutation signature and/or mutation spectrum is compared to empirically-derived genotoxin-associated information to determine (e.g., based on similarities and/or differences) a type of genotoxin the subject was exposed to (if not known), the mechanism of action of the genotoxin, a likelihood that the subject will develop a genotoxin-associated disease or disorder, and/or other genotoxin-associated information. For example, a Duplex Sequencing trinucleotide spectrum pattern resulting from a known or suspected genotoxin (e.g., the test genotoxin) exposure in a subject can be compared to empirically-derived trinucleotide spectrum patterns associated with exposure to other known genotoxins (e.g., such as stored in a database). In certain embodiments, the Duplex Sequencing trinucleotide spectrum pattern may be substantially similar to one or more of the empirically-derived trinucleotide spectrum patterns, such that a practitioner may be informed as to the identity of the test genotoxin, the level of exposure to the test genotoxin, the mechanism of action of the test genotoxin, etc. based on the similarity to the one or more empirically-derived trinucleotide spectrum patterns.

Mutant Frequency

In some embodiments, Duplex Sequencing analysis steps can identify a mutant frequency associated with a particular genotoxin under various exposure conditions. For example, a mutant frequency associated with an exposure of a biological sample to a genotoxin can vary depending on variety of factors including, but not limited to, organism/subject, age of subject, type of genotoxin, amount of time or level of exposure to a genotoxin, tissue type, treatment group, region of the genome (e.g., genomic locus), by type of mutation, by substitution type, and by trinucleotide context among other factors. In some examples, mutant frequency is measured as the number of unique mutations detected per duplex base-pair sequenced. In other embodiments, the mutant frequency is the rate of new mutations in a single gene or organism over time.

Mutation Spectrum

In various embodiments, the high accuracy (e.g., error-corrected) sequence reads generated using Duplex Sequencing can be further analyzed to generate a mutation spectrum or signature for a particular genotoxin or potential genotoxin. In one embodiment, a mutation spectrum or signature comprises the characteristic combinations of mutation types arising from mutagenic processes resulting from an exposure to a genotoxin. Such characteristic combinations can include information relating to the type of mutations (e.g., alterations to the nucleic acid sequence or structure). For example, a mutation spectrum can comprise a pattern information regarding the number, location and context of point mutations (e.g., single base mutations), nucleotide deletions, sequence rearrangements, nucleotide insertions, and duplications of the DNA sequence in the sample. In some embodiments a mutation spectrum may include information relevant to determine a mechanism of action resulting in the determined mutation patterns. For example, the mutation spectrum may be able to determine if mutagenic processes were directly caused by exogenous or endogenous genotoxin exposures or indirectly triggered by genotoxin exposure via perturbation of DNA replication infidelity, defective DNA repair pathways and DNA enzymatic editing, among others. In some embodiments, the mutation spectrum can be generated by computational pattern matching (e.g., unsupervised hierarchical mutation spectrum clustering, non-negative matrix factorization etc.).

Triplet Mutation Spectrum/Signature

In one embodiment, the high accuracy (e.g., error-corrected) sequence reads generated using Duplex Sequencing can be further analyzed to generate a triplet mutation spectrum (also referred to herein as a trinucleotide spectrum or signature). For example, the mutation spectrum associated with a genotoxin and/or with an incident of genotoxin exposure can be further analyzed to detect single nucleotide variations or mutations in a trinucleotide or trinucleotide context. Without being bound by theory, it is recognized that genotoxin exposure or other processes (e.g., aging) can cause variable and/or specific damage to nucleic acids depending on the trinucleotide context (e.g., a nucleotide base and its immediate surrounding bases). In some embodiments, a genotoxin can have a unique, semi-unique and/or otherwise identifiable triplet spectrum/signature. For example, a trinucleotide spectrum of a first genotoxin may predominantly include C⋅G→A⋅T mutations and may further have a higher predilection for CpG sites. Such a trinucleotide spectrum is similar proposed etiologies drive primarily by exposure to tobacco where Benzo[α]pyrene and other polycyclic aromatic hydrocarbons are known mutagens. In another example, urethane is a genotoxin that generates DNA damage in a periodic pattern of T⋅A→A⋅T in a 5′-NTG-3′ trinucleotide context. Accordingly, in some embodiments, determining a triplet mutation spectrum can be advantageous for identifying a genotoxin exposure in a subject, determining the genotoxicity of a potential genotoxin, and identifying a mechanism of action of a genotoxic agent or factor among other benefits.

Mechanism of Action

In some embodiments, the high accuracy (e.g., error-corrected) sequence reads generated using Duplex Sequencing can be used to infer the biochemical process(es) that result in the detected alterations to nucleic acid following exposure to a specific genotoxin. For example, in an embodiment, the mutant frequency and mutation spectrum (including the trinucleotide spectrum) generated using a Duplex Sequencing method can be compared to empirically-derived or a priori-derived information regarding the patterns and biochemical properties associated with observed mutation types as well as genomic location of the genetic mutation or DNA damage caused by the genotoxin exposure. In embodiments where the biochemical pathway and/or pathophysiological processes that follow the detected genomic pre-mutation, mutation or damage is ascertained, such information can be used, in some embodiments, to inform of treatment options (e.g., either therapeutic or prophylactic) for subjects exposed to the genotoxin, or in other embodiments, such information can be used to inform of viability of commercialization efforts (e.g., new drug), clean-up efforts (e.g., of an environmental toxin or manufacturing by-product), or in further embodiments, such information can be used to inform of a tested compound, agent or factor may be altered to eliminate and/or reduce the genotoxicity associated with the compound, agent or factor.

Sources of Nucleic Acid Material for Assessing Genotoxicity

As discussed above, it is contemplated that nucleic acid material may come from any of a variety of sources. For example, in some embodiments, nucleic acid material is provided from a sample from at least one subject (e.g., a human or animal subject) or other biological source. In some embodiments, a nucleic acid material is provided from a banked/stored sample. In some embodiments, a sample is or comprises at least one of blood, serum, sweat, saliva, cerebrospinal fluid, mucus, uterine lavage fluid, a vaginal swab, a nasal swab, an oral swab, a tissue scraping, hair, a finger print, urine, stool, vitreous humor, peritoneal wash, sputum, bronchial lavage, oral lavage, pleural lavage, gastric lavage, gastric juice, bile, pancreatic duct lavage, bile duct lavage, common bile duct lavage, gall bladder fluid, synovial fluid, an infected wound, a non-infected wound, an archeological sample, a forensic sample, a water sample, a tissue sample, a food sample, a bioreactor sample, a plant sample, a fingernail scraping, semen, prostatic fluid, fallopian tube lavage, a cell free nucleic acid, a nucleic acid within a cell, a metagenomics sample, a lavage of an implanted foreign body, a nasal lavage, intestinal fluid, epithelial brushing, epithelial lavage, tissue biopsy, an autopsy sample, a necropsy sample, an organ sample, a human identification ample, an artificially produced nucleic acid sample, a synthetic gene sample, a nucleic acid data storage sample, tumor tissue, and any combination thereof. In other embodiments, a sample is or comprises at least one of a microorganism, a plant-based organism, or any collected environmental sample (e.g., water, soil, archaeological, etc.). In particular examples discussed further herein, nucleic acid material may come from a biological source that has been exposed to a genotoxin or a potential genotoxin. In some examples, the genotoxin is a mutagen and/or a carcinogen. In an example, nucleic acid material is analyzed to determine if the biological source from which the nucleic acid material is derived was exposed to genotoxin.

When compared to other known or conventional toxicity assays, such as the Ames test (e.g., test for mutagenesis in bacteria), in vitro testing in mammalian cell culture, transgenic rodent assay, Pig-a assay, and the in vivo two-year bioassay, Duplex Sequencing provides multiple advancements. For example, many of the prior art methods are limited to interrogation of reporter genes as a surrogate for informative information relating to genotoxicity of a test agent/factor (e.g., Ames test, in vitro mammalian cell culture, in vivo transgenic rodent assay) or testing in non-human sources (e.g., Ames test, transgenic rodent assay, Pig-a assay, two-year bioassay), can require long periods of time to complete for very little information provided (e.g., two-year bioassay in wild-type rodents) or can be very costly (e.g., transgenic rodent assay, two-year bioassay). In contrast to many of the disadvantages of the prior art assays and techniques for screening test agents/factors for genotoxicity, Duplex Sequencing assays can be widely deployable, economical, suitable for both early and late screening of test agents/factors, utilized to provide high accuracy data in short periods of time (e.g., under 2 weeks), can be used to screen both in vitro and in vivo tested samples from any organism/biological source (i.e., including in vivo human samples among others) or any tissue/organ, evaluates multiple genetic loci and can use a natural genome as a reporter of genotoxicity and can inform on mechanism of action of a determined genotoxin agent/factor.

Kits with Reagents

Aspects of the present technology further encompass kits for conducting various aspects of Duplex Sequencing methods (also referred to herein as a “DS kit”). In some embodiments, a kit may comprise various reagents along with instructions for conducting one or more of the methods or method steps disclosed herein for nucleic acid extraction, nucleic acid library preparation, amplification (e.g. via PCR) and sequencing. In one embodiment, a kit may further include a computer program product (e.g., coded algorithm to run on a computer, an access code to a cloud-based server for running one or more algorithms, etc.) for analyzing sequencing data (e.g., raw sequencing data, sequencing reads, etc.) to determine, for example, a mutant frequency, mutation spectrum, triplet mutation spectrum, comparison to mutation spectrums of known genotoxins, etc., associated with a sample and in accordance with aspects of the present technology.

In some embodiments, a DS kit may comprise reagents or combinations of reagents suitable for performing various aspects of sample preparation (e.g., DNA extraction, DNA fragmentation), nucleic acid library preparation, amplification and sequencing. For example, a DS kit may optionally comprise one or more DNA extraction reagents (e.g., buffers, columns, etc.) and/or tissue extraction reagents. Optionally, a DS kit may further comprise one or more reagents or tools for fragmenting double-stranded DNA, such as by physical means (e.g., tubes for facilitating acoustic shearing or sonication, nebulizer unit, etc.) or enzymatic means (e.g., enzymes for random or semi-random genomic shearing and appropriate reaction enzymes). For example, a kit may include DNA fragmentation reagents for enzymatically fragmenting double-stranded DNA that includes one or more of enzymes for targeted digestion (e.g., restriction endonucleases, CRISPR/Cas endonuclease(s) and RNA guides, and/or other endonucleases), double-stranded Fragmentase cocktails, single-stranded DNase enzymes (e.g., mung bean nuclease, S1 nuclease) for rendering fragments of DNA predominantly double-stranded and/or destroying single-stranded DNA, and appropriate buffers and solutions to facilitate such enzymatic reactions.

In an embodiment, a DS kit comprises primers and adapters for preparing a nucleic acid sequence library from a sample that is suitable for performing Duplex Sequencing process steps to generate error-corrected (e.g., high accuracy) sequences of double-stranded nucleic acid molecules in the sample. For example, the kit may comprise at least one pool of adapter molecules comprising single molecule identifier (SMI) sequences or the tools (e.g., single-stranded oligonucleotides) for the user to create it. In some embodiments, the pool of adapter molecules will comprise a suitable number of substantially unique SMI sequences such that a plurality of nucleic acid molecules in a sample can be substantially uniquely labeled following attachment of the adapter molecules, either alone or in combination with unique features of the fragments to which they are ligated. One experienced in the art of molecular tagging will recognize that what entails a “suitable” number of SMI sequences will vary by multiple orders of magnitude depending on various specific factors (input DNA, type of DNA fragmentation, average size of fragments, complexity vs repetitiveness of sequences being sequenced within a genome etc.) Optionally, the adaptor molecules further include one or more PCR primer binding sites, one or more sequencing primer binding sites, or both. In another embodiment, a DS kit does not include adapter molecules comprising SMI sequences or barcodes, but instead includes conventional adapter molecules (e.g., Y-shape sequencing adapters, etc.) and various method steps can utilize endogenous SMIs to relate molecule sequence reads. In some embodiments, the adapter molecules are indexing adapters and/or comprise an indexing sequence.

In an embodiment, a DS kit comprises a set of adapter molecules each having a non-complementary region and/or some other strand defining element (SDE), or the tools for the user to create it (e.g., single-stranded oligonucleotides). In another embodiment, the kit comprises at least one set of adapter molecules wherein at least a subset of the adapter molecules each comprise at least one SMI and at least one SDE, or the tools to create them. Additional features for primers and adapters for preparing a nucleic acid sequencing library from a sample that is suitable for performing Duplex Sequencing process steps are described above as well as disclosed in U.S. Pat. No. 9,752,188, International Patent Publication No. WO2017/100441, and International Patent Application No. PCT/US18/59908 (filed Nov. 8, 2018), all of which are incorporated by reference herein in their entireties.

Additionally, a kit may further include DNA quantification materials such as, for example, DNA binding dye such as SYBR™ green or SYBR™ gold (available from Thermo Fisher Scientific, Waltham, Mass.) or the alike for use with a Qubit fluorometer (e.g., available from Thermo Fisher Scientific, Waltham, Mass.), or PicoGreen™ dye (e.g., available from Thermo Fisher Scientific, Waltham, Mass.) for use on a suitable fluorescence spectrometer. Other reagents suitable for DNA quantification on other platforms are also contemplated. Further embodiments include kits comprising one or more of nucleic acid size selection reagents (e.g., Solid Phase Reversible Immobilization (SPRI) magnetic beads, gels, columns), columns for target DNA capture using bait/pray hybridization, qPCR reagents (e.g., for copy number determination) and/or digital droplet PCR reagents. In some embodiments, a kit may optionally include one or more of library preparation enzymes (ligase, polymerase(s), endonuclease(s), reverse transcriptase for e.g., RNA interrogations), dNTPs, buffers, capture reagents (e.g., beads, surfaces, coated tubes, columns, etc.), indexing primers, amplification primers (PCR primers) and sequencing primers. In some embodiments, a kit may include reagents for assessing types of DNA damage such as an error-prone DNA polymerase and/or a high-fidelity DNA polymerase. Additional additives and reagents are contemplated for PCR or ligation reactions in specific conditions (e.g., high GC rich genome/target).

In an embodiment, the kits further comprise reagents, such as DNA error correcting enzymes that repair DNA sequence errors that interfere with polymerase chain reaction (PCR) processes (versus repairing mutations leading to disease). By way of non-limiting example, the enzymes comprise one or more of the following: Uracil-DNA Glycosylase (UDG), Formamidopyrimidine DNA glycosylase (FPG), 8-oxoguanine DNA glycosylase (OGG1), human apurinic/apyrimidinic endonuclease (APE 1), endonuclease III (Endo III), endonuclease IV (Endo IV), endonuclease V (Endo V), endonuclease VIII (Endo VIII), N-glycosylase/AP-lyase NEIL 1 protein (hNEIL1), T7 endonuclease I (T7 Endo I), T4 pyrimidine dimer glycosylase (T4 PDG), human single-strand-selective monofunctional uracil-DNA glycosylase (hSMUG1), human alkyladenine DNA glycosylase (hAAG), etc.; and can be utilized to correct DNA damage (e.g., in vitro DNA damage). Some of such DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., most common DNA lesion that results from reactive oxygen species). FPG also has lyase activity that can generate 1 base gap at abasic sites. Such abasic sites will subsequently fail to amplify by PCR, for example, because the polymerase fails copy the template. Accordingly, the use of such DNA damage repair enzymes, and/or others listed here and as known in the art, can effectively remove damaged DNA that does not have a true mutation but might otherwise be undetected as an error following sequencing and duplex sequence analysis.

The kits may further comprise appropriate controls, such as DNA amplification controls, nucleic acid (template) quantification controls, sequencing controls, nucleic acid molecules derived from a biological source exposed to a known genotoxin/mutagen (e.g., DNA extracted from a test animal or cells grown in culture that were exposed to the genotoxin) and/or nucleic acid molecules derived from a biological source that was not exposed to a genotoxin/mutagen. In another embodiment, the control reagents may include nucleic acid that has been intentionally damaged and/or nucleic acid that has not been damaged or exposed to any damaging agent. In additional embodiments, a kit may also include one or more genotoxic and/or non-genotoxic agents (e.g., compounds) to be delivered in a controlled genotoxicity experiment, and optionally include protocols for delivering such agents to a subject, tissue, cell, etc. Accordingly, a kit could include suitable reagents (test compounds, nucleic acid, control sequencing library, etc.) for providing controls that would yield duplex sequencing results (e.g., an expected mutation spectrum/signature) that would determine protocol authenticity for a test substance (e.g., test compound, potential genotoxic agent or factor, etc.). In an embodiment, the kit comprises containers for shipping subject samples, such as blood samples, for analysis to detect mutations in a subject sample, the pattern and type thus indicating which genotoxins the subject has been exposed to. In another embodiment, a kit may include nucleic acid contamination control standards (e.g., hybridization capture probes with affinity to genomic regions in an organism that is different than the test or subject organism).

The kit may further comprise one or more other containers comprising materials desirable from a commercial and user standpoint, including PCR and sequencing buffers, diluents, subject sample extraction tools (e.g. syringes, swabs, etc.), and package inserts with instructions for use. In addition, a label can be provided on the container with directions for use, such as those described above; and/or the directions and/or other information can also be included on an insert which is included with the kit; and/or via a website address provided therein. The kit may also comprise laboratory tools such as, for example, sample tubes, plate sealers, microcentrifuge tube openers, labels, magnetic particle separator, foam inserts, ice packs, dry ice packs, insulation, etc.

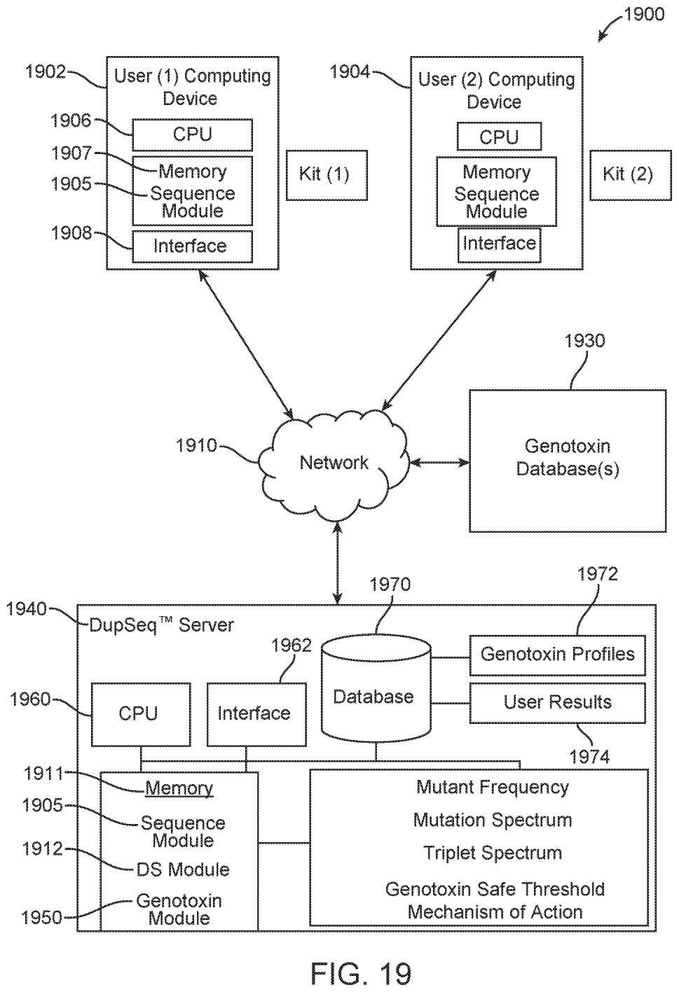

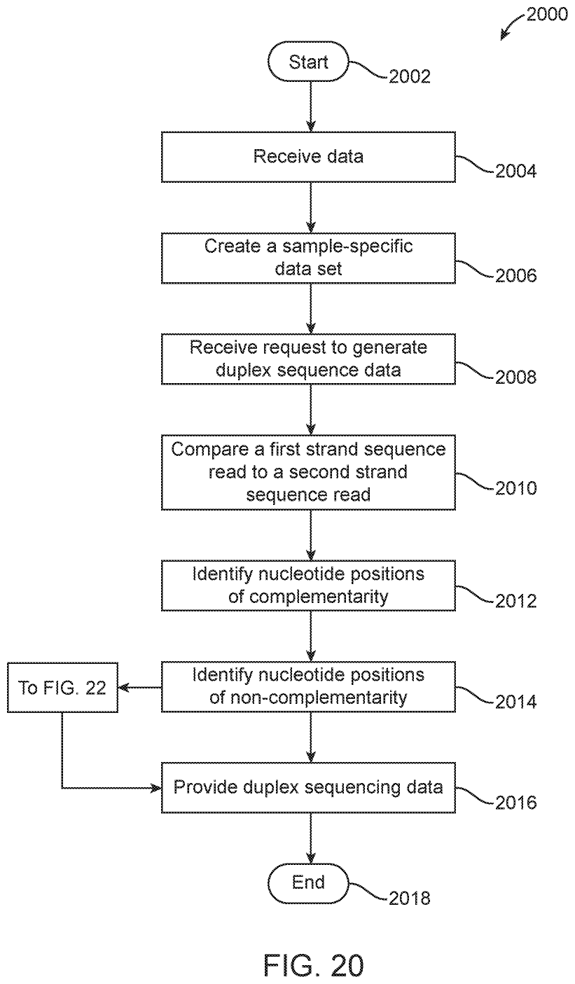

The kits may further comprise a computer program product installable on an electronic computing device (e.g. laptop/desktop computer, tablet, etc.) or accessible via a network (e.g. remote server), wherein the computing device or remote server comprises one or more processors configured to execute instructions to perform operations comprising Duplex Sequencing analysis steps. For example, the processors may be configured to execute instructions for processing raw or unanalyzed sequencing reads to generate Duplex Sequencing data. In additional embodiments, the computer program product may include a database comprising subject or sample records (e.g., information regarding a particular subject or sample or groups of samples) and empirically-derived information regarding known genotoxins). The computer program product is embodied in a non-transitory computer readable medium that, when executed on a computer, performs steps of the methods disclosed herein (e.g. see FIGS. 19 and 20).

The kits may further comprise include instructions and/or access codes/passwords and the like for accessing remote server(s) (including cloud-based servers) for uploading and downloading data (e.g., sequencing data, reports, other data) or software to be installed on a local device. All computational work may reside on the remote server and be accessed by a user/kit user via internet connection, etc.

### High Throughput Genotoxin Screening

The present technology further comprises high throughput screening schemes for assessing genotoxicity of suspected agents or factors (e.g., a compound, chemical, pharmaceutical agent, manufacturing product or by-product, food substance, environmental factor, etc.). In one embodiment, an agent/factor having an unknown genotoxicity effect can be screened to determine whether the test agent/factor comprises a genotoxic effect. In some embodiments, agents/factors can be screened with a desire to eliminate use of agents/factors that have a genotoxic effect or exceed a threshold genotoxic effect. For example, an agent/factor that is mutagenetic in a manner that can potentially cause a genotoxicity-associated disease or disorder can be identified such that the agent/factor can be properly controlled, eliminated, discarded, stored, etc. In some embodiments, agents/factors that are carcinogenic can be identified using high throughput screening schemes as described herein. In another embodiment, an agent/factor having an unknown genotoxicity effect can be screened with an intent to discover an agent/factor that has a desired genotoxic effect, and in particular a desired genotoxic effect on a target biological source. For example, biological samples derived from a patient having a disease or disorder (e.g., cancer) can be used in a high throughput screening scheme to test multiple agents/factors for a desired genotoxic effect, that may result in perturbing or destroying the cell (e.g., cancer cell). Such screening can be performed for discovery of new drugs/therapies and/or for targeted therapies for use in personalized medicine.

In some embodiments, high throughput screening refers to screening a plurality of samples simultaneously and/or time-efficiently. In one example, testing an agent or factor for genotoxicity comprises exposing (e.g., treating, administering, applying, etc.) a subject (e.g., a biological source) to a test agent or factor. Accordingly, for high through-put screening schemes, an array of biological sources/samples can be treated simultaneously with the same test agent/factor, or in other embodiments, with multiple test agents/factors. In a particular example, a plurality of biological samples (e.g., human or other organism cells grown in culture, tissue samples, blood or other bodily fluid samples, transgenic animal's cells, human cells grown in xenografts, live patient organoids, feeder cells, etc.) can be exposed to a test agent/factor substantially simultaneously and under consistent conditions. High throughput screening may also be used via organs-on-chips, such as using a 10-organ chip with blood or tissue samples from the same subject extracted from the following organs and tissues: endocrine; skin; GI-tract; lung; brain; heart; bone marrow; liver; kidney; and pancreas. Methods of use of organs-on-chips for high throughput screening are well known in the art (e.g. Chan et al. [5]). In other embodiments, genetically modified cell lines (e.g., having deficient or impaired DNA repair pathways to make such cells more sensitive to mutagenic or genotoxic damage effects) can be incorporated into a high throughput screening scheme.

In some embodiments, the plurality of biological samples can be the same or substantially similar (e.g., identical cell lines grown in culture, tissue samples from the same subject and/or same tissue type, etc.). In other embodiments, one or more of the plurality of biological samples can be different. For example, a test agent/factor can be tested for a genotoxic effect on different tissue/cell types from the same organism, a different organism or a combination thereof. In a particular example, a suspected genotoxic agent or factor (e.g. a compound, a pharmaceutical drug, etc.) can be tested concurrently on tissue samples from various organs of the same subject (e.g. a 10-organ chip). In some embodiments, high throughput screening can encompass testing multiple test agents/factors simultaneously. Accordingly, it is contemplated that each tested sample can have different properties that can intentionally vary or not (e.g., by cell type, by tissue type, by subject from which a cell or tissue is extracted, by species, etc.) and/or be subjected to different testing regimes that can vary per design (e.g., by test agent/factor, by dose level, by time of exposure, etc.) such that a high throughput screening scheme can be used to efficiently screen multiple samples in a manner that provides any desired information.

Once the biological samples are exposed and/or a desired exposure regime is completed, cells/tissue from the samples can be harvested and DNA can be extracted for the purpose of using Duplex Sequencing to assess the test agent/factor's genotoxic/mutagenic impact on the DNA derived from each sample. In some embodiments, cell-free DNA (such as released in culture media) can be collected from the biological samples for Duplex Sequencing analysis. Further embodiments contemplated by the present technology include high throughput processing of DNA samples to generate Duplex Sequencing data for assessing DNA damage, mutagenicity or carcinogenicity of a known or suspected genotoxin.

The high throughput screening processes described herein may comprise automation, such as via the use of robotics for performing one or more of experimental treatment of biological samples, DNA extraction, library preparation steps, amplification steps (e.g., PCR) and/or DNA sequencing steps (e.g., using various techniques and devices for massively parallel sequencing). Using high throughput screening allows a plurality of samples (i.e. different cell types from the same subject, or the same cell types from different subjects) to be tested in parallel so that large numbers of samples are quickly screened for genotoxic-associated mutations and/or DNA damage.

In an embodiment, microplates, each of which consists of an array of wells, each well comprising one sample, are moved through the system by robotic handling. In an example, the wells in the microplates can be filled via automated liquid handling systems, and sensors can be used to evaluate the samples in the microplate, e.g., often after a period of incubation. Laboratory automation software can be used to control the entire or a portion of the screening process, thereby ensuring accuracy within the process and repeatability between processes.

### Environmental/Exogenous Genotoxins

Aspects of the present technology comprise assessing genotoxicity of environmental/exogenous agents/factors, such as by using any of the above described in vivo or in vitro Duplex Sequencing screening methods. Additional aspects of the present technology comprise assessing whether subjects/organisms have been exposed to a genotoxin in an environmental area. For example, biological samples (e.g., tissue, blood) can be collected from organisms living or otherwise exposed to a suspected area of contamination to, e.g., determine if an area is contaminated. In other embodiments, biological samples can be collected from organisms present in a larger area and assessed as a screening process to pin-point a specific geographical location of a source of a genotoxin contamination (e.g., industrial by-product leaked/released into a water system). Various methods as described herein can be used to analyze biological samples (e.g., from subjects) exposed to an environmental area that is under investigation for the presence of a possible genotoxin. In another embodiment, various methods as described herein can be used to analyze biological sample(s) taken from subject that is suspected of being exposed to a known genotoxin in an environmental area (e.g., a geographical area, a living area, an occupational environment, etc.). In accordance with aspects of the present technology, biological samples can be sourced from multiple organisms (e.g., sea-life, mammal, filter feeder, sentinel organism, etc.) or a specific species (e.g., human samples).

Detectable environmental genotoxins further comprise exposure to one or more of mutagenic agents, such as, but not limited to, gamma-irradiation, X-rays; UV-irradiation; microwaves; electronic emissions; poisonous gas; poisonous air particulates (e g inhaling asbestos); and chemical compound and/or pathogen contaminated lakes, rivers, streams, groundwater, etc. Additional sources of exogenous genotoxins can include, for example, food substances, cosmetics, house-hold items, health-care related products, cooking products and tools, and other manufactured consumables.

The Duplex Sequencing results may further be used in conjunction with other methods of identifying the presence of disease-causing contaminants, such as an epidemiological study first identifying the location of a cancer cluster. In some embodiments, methods disclosed herein can be utilized to identify the specific genotoxins that affected members of the cluster. From this data, the source of the genotoxin can be determined. In contrast to conventional means of investigation which have traditionally used correlative information to link a disease or medical condition of a subject to a causative event (e.g., exposure to an environmental or other exogenous mutagen or carcinogen), Duplex Sequencing provides high accuracy, reproducible data, such as mutation spectrum and mechanism of action, which results can be used to empirically determine the causative event(s) (e.g., exposure to a specific mutagen or carcinogen).

### Endogenous Genotoxins

Aspects of the present technology comprise assessing genotoxicity of endogenous agents/factors (e.g., an endogenous genotoxin or genotoxic process), such as by using any of the above described in vivo or in vitro Duplex Sequencing screening methods. Accordingly, aspects of the present technology comprise assessing whether subjects/organisms have experienced an endogenous genotoxin or genotoxic process that has caused DNA damage. For example, biological samples (e.g., tissue, blood) can be collected from a subject (e.g., a patient) to, e.g., determine if the subject has a genotoxin-associated disease or disorder or is at-risk of developing such a disease or disorder.

Endogenous factors may comprise, by way of non-limiting examples: biological incidents causing misincorporation of nucleotides, such as DNA polymerase errors, free radicals, and depurination. Endogenous factors may further comprise the onset of biological conditions, short or long term, that directly contribute to disease or disorder associated polynucleotide mutation, such as, for example, stress, inflammation, activation of an endogenous virus, autoimmune disease; environmental exposures; food choices (e.g. carcinogenic foods and drink); smoking; natural genetic makeup; aging; neurodegeneration; and so forth. For example, if a subject is exposed long term to high levels of stress, the subject can be tested via Duplex Sequencing for any mutation that is correlated with stress-associated cancers (e.g. leukemia, breast cancer, etc.).

Endogenous factors may also represent the aggregate accumulation of mutations and other genotoxic events in the tissues of an individual human that reflect the integral effects of the individual's exposures and may not be able to be precisely quantified or experimentally controlled.

### Methods for Determining Safe Mutant Frequency Levels

A level or amount of DNA damage resulting from an exposure to a genotoxin can vary depending on a variety of factors including, for example, effectiveness of a genotoxin at causing DNA damage (either directly or indirectly), dose or amount of exposure, route or manner of exposure (e.g., ingested, inhaled, transdermal absorption, intravenous, etc.), duration (e.g., over time) of exposure, synergistic or antagonistic effects of other agents or factors to which the subject is exposed, in addition to various characteristics of the subject (e.g., level of health, age, gender, genetic makeup, prior genotoxin exposure events, etc.). As discussed above, exposure to a genotoxin can result in polynuclear acid damage that can be assessed, e.g., by Duplex Sequencing methods as described herein, to determine a unique, semi-unique and/or otherwise identifiable mutagenic spectrum or signature associated with the that may comprise a mutation pattern (e.g. mutation type, mutant frequency, identifiable mutations in a trinucleotide context) sufficiently similar to a known disease-associated mutation pattern (e.g. a distinct genomic mutation for breast cancer). Various aspects of the present technology are directed to methods for determining and/or quantifying mutant frequency levels that can be considered safe further comprise a method of detecting a safe threshold mutant frequency for a genotoxin. When the mutant frequency within the sample is above a safe level, then it indicates that the subject is at a significantly increased risk of developing the disease over time.

The present technology further comprises a method for detecting and quantifying genomic mutations developed in vivo in a subject following the subject's exposure to a mutagen, comprising: (1) duplex sequencing one or more target double-stranded DNA molecules extracted from a subject exposed to a mutagen; (2) generating an error-corrected consensus sequence for the targeted double-stranded DNA molecules; and (3) identifying a mutation spectrum for the targeted double-stranded DNA molecules; (4) calculating a mutant frequency for the target double-stranded DNA molecules by calculating the number of unique mutations per duplex base-pair sequenced. In an embodiment of step (3), the mutation spectrum is a sample's unique profile comprises a “trunucleotide signature”.

In an embodiment, steps (1) and (2) are accomplished by: a) ligating the double-stranded target nucleic acid molecule to at least one adapter molecule, to form an adaptor-target nucleic acid complex, wherein the at least one adaptor molecule comprises: i. a degenerate or semi-degenerate single molecule identifier (SMI) sequence that alone or in combination with the target nucleic acid shear points uniquely labels the double stranded target nucleic acid molecule; and ii. a nucleotide sequence that tags each strand of the adaptor-target nucleic acid complex such that each strand of the adaptor-target nucleic acid complex has a distinctly identifiable nucleotide sequence relative to its complementary strand, b) amplifying each strand of the adaptor-target nucleic acid complex to produce a plurality of first strand adaptor-target nucleic acid complex amplicons and a plurality of second strand adaptor-target nucleic acid complex amplicons; c) sequencing the adaptor-target nucleic acid complex amplicons to produce a plurality of first strand sequence reads and a plurality of second strand sequence reads; and d) comparing at least one sequence read from the plurality of first strand sequence reads with at least one sequence read from the plurality of second strand sequence reads and generating an error corrected sequence read of the double stranded target nucleic acid molecule by discounting nucleotide positions that do not agree (see U.S. Pat. No. 9,752,188 B2, and WO 2017/100441).

### Methods of Determining Safe Threshold Levels of Genotoxin Amount

The present technology further comprises experimental in vitro and in vivo methods for determining safe levels (concentration amounts by weight or volume or mass or unit*time integrals etc.) of exposure by a subject to a specific genotoxin; and/or whether or not a compound or other agent (e.g. radio waves from wireless device etc.) is genotoxic at any level of exposure. This determination may depend on first determining the safe threshold mutant frequency level. In an embodiment, a control subject's sample is tested for genotoxins (or lack thereof) and compared to the genotoxin profile of exposed subjects' samples (e.g. a plurality of mice; or a plurality of cells from the same subject, one set of which are the control cells; etc.). The exposed subjects receive designated, predetermined exposure amounts of suspected genotoxin to determine the threshold level of safe exposure before a detected genotoxin induced mutation occurs that directly contributes to disease onset.

In another embodiment, test subject's (e.g. lab animals, in vitro cells, etc.) are exposed to different doses for different time periods, and from which it is determined the safe cutout level of genotoxin exposure: 1) at what dose of exposure no polynucleotide mutations are seen: and/or 2) at what dose of exposure are polynucleotide mutations detected, but where dose equivalent level does not cause cancer in subjects, and using the level of mutations found to infer the same of other compounds; and/or 3) determining a genotoxin dose response curve and regression analysis of induced mutations to extrapolate a linear low dose response curve; and/or 4) what the hazard ratio for a given health outcome in a subject population is that is associated with a detected genotoxin frequency/signature detected.

The threshold levels of safe exposure may further be determined by species—e.g. human, dog/cat, horse, etc. The safe threshold levels may further be determined by routes of exposure to the genotoxin. For example, experiments using various amounts of genotoxins can be tested with the Duplex Sequencing methods disclosed herein to determine the amount (weight, volume, etc.) and/or frequency by oral, topical, or aerosol consumption that would result in a mutation and triplet spectrum associated with a specific disease development.

And/or the Duplex Sequencing experimental methods disclosed herein can be used to determine the threshold amount of genotoxic exposure based on time and/or temperature. For example, absorption through the skin from a shower or a bath in water containing a genotoxin based on the duration of exposure, and temperature of the water, and concentration of the genotoxin in the water, can be used to compute the amount (dose) of genotoxin absorbed through the skin.

The error-corrected Duplex Sequencing results identifying genotoxin safe threshold levels may further be combined with other safety threshold data (e.g. existing FDA and EPA levels, Agency for Toxic Substance Disease Registry levels, the US National Toxicology Program guidelines, OECD guidelines, Canadian Health guidelines, European regulatory guidelines, ILSI/HESI guidelines etc.) to affirm or adjust the established standards

### Methods of Detection and Treatment

Disease or disorder onset may not be able to be diagnosed via traditional testing and imaging techniques until many years after genotoxin exposure (e.g. 20 years); but the present technology provides methods of detecting the disease-causing mutations, or indication of genotoxic processes with the potential to cause disease-causing mutations or precursors to mutations, within a few days or a few weeks or a few months following genotoxin exposure in order to prophylactically treat the subject, or actively screen the subject for disease (by virtue of being at a higher risk level), as well as identify the presence of a genotoxin and eliminate it to prevent future exposures.

When a subject is exposed to more than a genotoxin's threshold safe level and/or when it has been determined that a subject has potentially been exposed to unsafe levels of a genotoxin (e.g. health department identifying dangerous levels of exposure), then the subject is at a significantly increased risk for the onset of the genotoxic associated disease or disorder. The subject is then treated prophylactically with agents that block and/or counteract the genotoxin; and/or the genotoxin exposure is reduced or eliminated (e.g. removing the genotoxin from the environment, or moving the subject). Additionally, or alternatively, the subject undergoes sequentially timed diagnostic testing (e.g. blood test for cancer detection) and/or imaging (e.g. CAT, MRI, PET, ultrasound, serum biomarker testing, etc.) to detect whether the subject has developed an early stage of the disease or disorder, during which time it is most effectively treated. By way of non-limiting example: for aflatoxin or aristolochoic acid exposure, the subject would likely be ordered to undergo a liver ultrasound every 6 months, the typical schedule on which patients with chronic hepatitis C, another hepatocarcinogen, are screened for hepatocellular carcinomas. At the time that traditional diagnostic tests well known in the art detect the disease (e.g. cancer), then treatment is initiated (e.g. surgery, chemotherapy, immunotherapy etc.).

Methods of providing prophylactic treatments (i.e. prevent or reduce the risk of onset), and/or to inhibit the growth of cancer, and/or to eradicate the cancer comprise treatment protocols well known to the skilled clinician, and would be tailored to the genotoxin type. Although treatments do not currently exist to reverse mutations that have already been induced, therapeutic methods for helping a subject clear certain residual genotoxins (for example, particular heavy metals via chelation), may decrease further genotoxicity.

For tumors that are mutagen induced (e.g. lung cancer in smoker, melanoma in the heavily UV-exposed, oral cancers in tobacco users etc.), the burden of mutations in these tumors tends to be higher, which is believed to lead to a greater abundance of neoantigens, and explain their far greater tendency to respond favorably to immunotherapies. It is probable that prophylactic administration of immunotherapies, such as those comprising checkpoint inhibitors (i.e. PD1 and PDL1 inhibitors such as nivolumab, pembrolizumab and atezolizumab, CTLA4 inhibitors such as ipilizumab) to enable the subject's immune system to eradicate early forming tumors. Hence, another treatment-directed use of identification of an exposure signature is the prediction of future tumor responsiveness to immunotherapy and potentially even disease prevention with prophylactic treatment, albeit requiring careful testing in the setting of formal clinical trials.

Methods of detection and treatment may further comprise methods of directly or inferentially determining the mechanism of action of the genotoxin, which may be used in determining the appropriate course of treatment; and/or monitoring for drug resistant variants (see Schmitt et al [6]).

Once the subject is diagnosed or detected to have been exposed to at least one genotoxin, the subject may be administered a therapeutically effective amount of a pharmaceutical composition to prevent onset, delay onset, reduce the effects of, and/or eradicate the genotoxin associated disease or disorder. A pharmaceutical composition comprises a therapeutically effective amount of a composition comprising an inhibitor or eradicator of a genotoxin associated disease or disorder, and a pharmaceutically acceptable carrier or salt. And a therapeutically effective amount comprises the therapeutic, non-toxic, dose range of the composition comprising an inhibitor or eradicator of a genotoxin associated disease or disorder, effective to produce the intended pharmacological, therapeutic or prophylactic result.

The pharmaceutical composition is formulated for, and administered by, a route of administration comprising: oral, intravenous, intramuscular, subcutaneous, intraurethral, rectal, intraspinal, topical, buccal, or parenteral administration. The pharmaceutical composition can be mixed with conventional pharmaceutical carriers and excipients and used in the form of tablets, capsules, pills, liquids, intravenous solutions, drink and food products, and the like; and will contain from about 0.1% to about 99.9%, or about 1% to about 98%, or about 5% to about 95%, or about 10% to about 80%, or about 15% to about 60%, or about 20% to about 55% by weight or volume of the active ingredient.

For oral administration, the tablets, pills, and capsules may additionally conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica or talc: disintegrants, for example, potato starch, flavoring or coloring agents, or acceptable wetting agents. Oral liquid preparations may be formulated into aqueous or oily solutions, suspensions, emulsions, syrups or elixirs and may contain conventional additives such as suspending agents, emulsifying agents, non-aqueous agents, preservatives, coloring agents and flavoring agents.

For intravenous routes of administration, the pharmaceutical composition can be dissolved or suspended in any of the commonly used intravenous fluids and administered by infusion. Intravenous fluids include, without limitation, physiological saline or Ringer's solution.

Pharmaceutical compositions for parental administration may be in the form of aqueous or non-aqueous isotonic sterile injection solutions or suspensions. These solutions or suspensions can be prepared from sterile powders or granules having one or more of the carriers mentioned for use in the formulations for oral administration. The compounds can be dissolved in polyethylene glycol, propylene glycol, ethanol, corn oil, benzyl alcohol, sodium chloride, and/or various buffers.

The therapeutic effect dose may further be computed based on a variety of factors, such as: amount or duration of genotoxic exposure; age, weight, sex or race of the subject; stage of development of the disease or disorder; and other methods well known to the skilled clinician. In an embodiment, the subject is tested upon discovery of their potential or suspected exposure to a genotoxin, even if the exposure occurred many years prior. If diagnosed as being exposed above a safe threshold level, then the subject is administered the pharmaceutical compound immediately or upon the display of symptoms. In all embodiments, the genotoxin is removed from the subject's environment when possible.

### Experimental Exampes

The following section provides examples of methods for detecting and assessing genomic in vivo mutagenesis using Duplex Sequencing and associated reagents. The following examples are presented to illustrate the present technology and to assist one of ordinary skill in making and using the same. The examples are not intended in any way to otherwise limit the scope of the technology.

Generally, to benchmark the efficacy of DS for measuring in vivo mutagenesis, a series of mouse experiments that generated 8.2 billion error-corrected bases across 62 samples was performed to examine the effect of three mutagens on nine genes from five healthy tissues in two independent animal strains. Duplex Sequencing quantitatively demonstrated an increased mutant frequency among treated animals, to an extent that varied by specific mutagen, tissue type and genomic locus, and closely mirrored that of a gold-standard transgenic rodent assay. In various examples, it was possible to identify samples by their treatment group based on objective mutational patterns alone. In some examples, mutagen sensitivity varied up to four-fold among different genic loci, and, without being bound by theory, spectral patterns suggested this to be partially the result of regionally distinct processes, which may include transcription and methylation. In various examples, the trinucleotide mutational signature among SNVs identified by DS at ultralow frequency in animals treated with the tobacco-related carcinogen benzo[a]pyrene, was shown to be almost identical to that seen among clonal SNVs in the genomes of smoking-associated lung cancers in publicly available databases. In some examples, DS was used to identify low-frequency oncogenic driver mutations clonally expanding under selective pressure, merely 4 weeks following a mutagen treatment. Accordingly, and as demonstrated in various examples described herein, DS can be used for directly quantifying both genotoxic processes and real-time neoplastic evolution, with diverse applications in mutational biology, toxicology and cancer risk assessment.

### Example 1

Application of Duplex Sequencing for in vivo mutation analysis in the cII transgene and endogenous genes in BigBlue® Mice. This section describes an example wherein error-corrected Next Generation Sequencing (NGS) was used to directly measure chemically-induced mutations in both the cII transgene used in the BigBlue® transgenic rodent (TGR) mutation assay, and in native mouse genes. Currently, TGR mutation assays detect rare cII mutants through plaque formation. Standard NGS is unusable for low-frequency mutation detection due to its high error rate (˜1 error per 10³ bases sequenced). Error-corrected NGS, or Duplex Sequencing, has a drastically lower error rate (˜ 1/10⁸ bases), permitting detection of ultra-rare mutations.

In this example, an application of Duplex Sequencing was used to evaluate mutant frequency (MF) and spectrum in control, N-ethyl-N-nitrosourea (ENU) and Benzo[a]pyrene (B[a]P)-exposed BigBlue® C57BL6 male mice.

BigBlue® transgenic C57BL/6 male mice were treated by daily oral gavage with vehicle (olive oil) or B[a]P (50 mg/kg/day) on Days 1-28, or with ENU (40 mg/kg/day in pH 6 buffer) on Days 1-3 (n=6). Tissues were collected and frozen on study day 31. Liver and bone marrow were analyzed for mutants. DNA was isolated and mutants analyzed for cII mutant plaques using RecoverEase and Transpack methods described by Agilent Technologies. Duplex Sequencing was used to sequence cII and other endogenous genes for mutations in liver and bone marrow.

Genes evaluated and criteria used to select genes are as follows: (1) Polr1c (RNA polymerase), which is ubiquitously transcribed in all tissue types; (2) Rho (Rhodopsin), which is not expressed in any tissue besides retina; (3) Hp (Haptoglobin), which is highly expressed in liver, but almost nowhere else; (4) Ctnnb1 (Beta-catenin), which is most commonly mutated gene in human hepatocellular carcinoma; and (5) CII: 360 bp transgenic reporter gene present in ˜80 copies in BigBlue® mice.

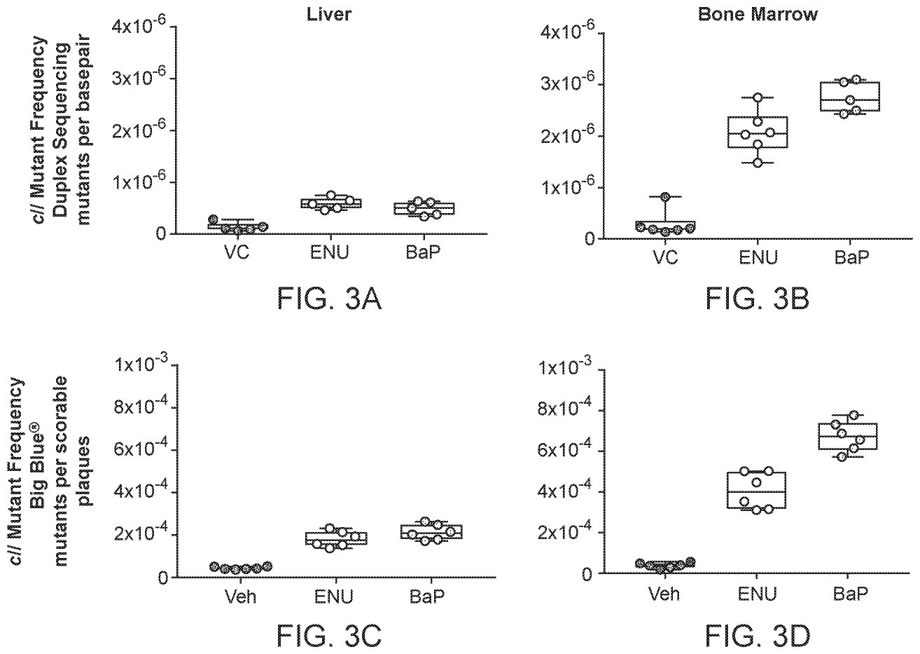

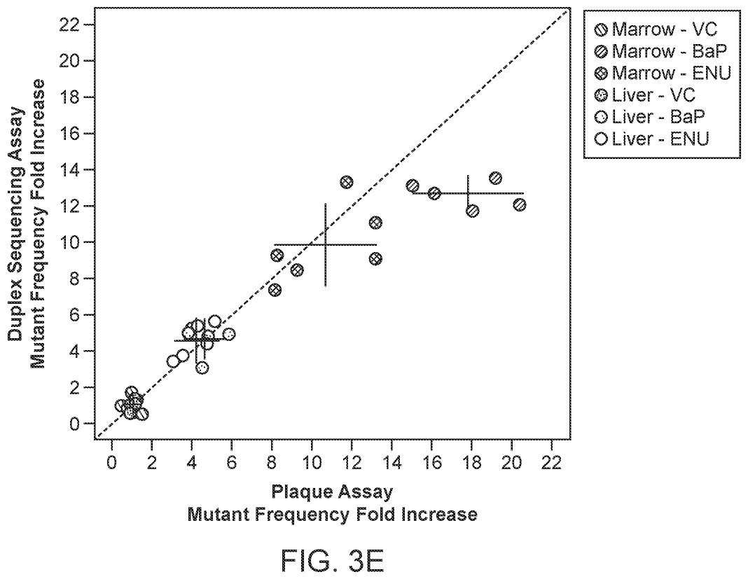

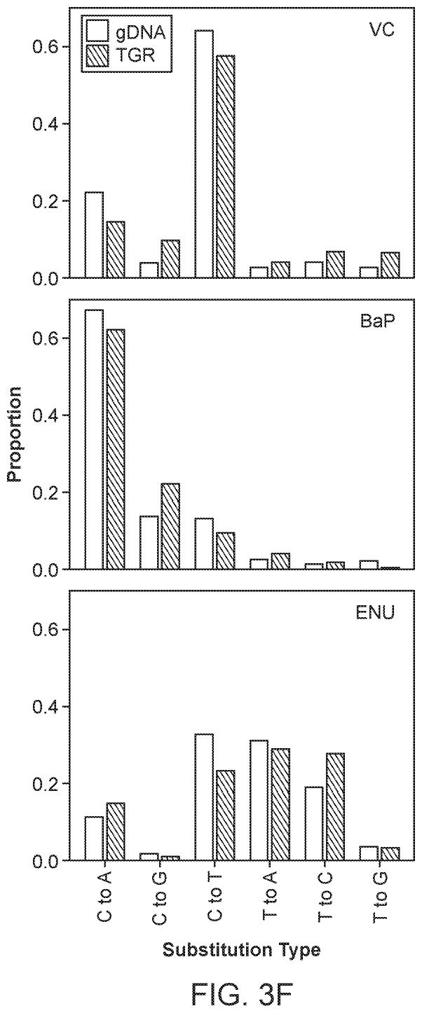

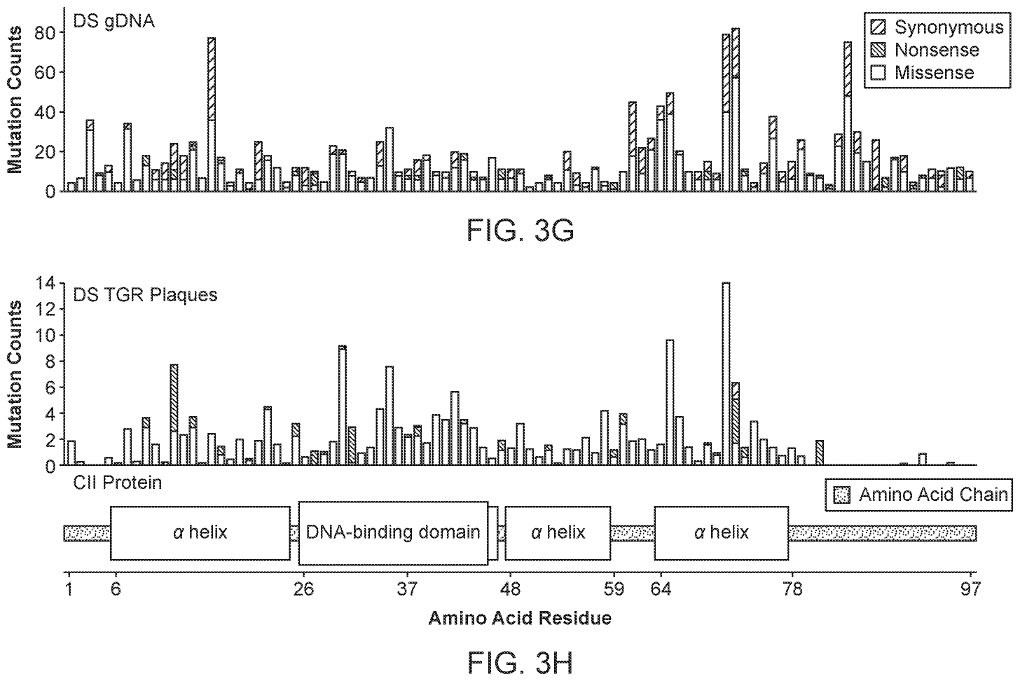

FIGS. 3A-3D are box plot graphs showing mutant frequencies calculated for Duplex Sequencing (FIGS. 3A and 3B) and the BigBlue® cII plaque assay (FIGS. 3C and 3D) in liver and bone marrow following mutagen treatment as described above. MF for Duplex Sequencing was based on total mutants per duplex base-pair sequenced (n=5 mice/group). MF for BigBlue® was calculated as number of mutant plaques relative to number of mutant plaque forming units (n=6 mice/group). As shown, MF measured by Duplex Sequencing and the traditional BigBlue® cII plaque assay gave similar responses to both mutagens. Bone marrow, which has faster dividing cells, demonstrated higher MF than liver using both methods.

FIG. 3E illustrates the relative cII mutant fold increase in the transgenic rodent assay vs Duplex Sequencing. As above, MF in the plaque assay is calculated as the number of phenotypically active mutant plaques observed on a selection plate divided by the total number of plaques formed on a permissive plate. MF in the Duplex Sequencing assay is calculated as the number of mutant base pair observations divided by the total number of base pairs sequenced within the 297 BP cII transgene interval. Despite differences in derivative measurements, correlation between the Duplex Sequencing assay and the BigBlue® cII plaque assay is strong across tissues and mutagen treatments.

FIG. 3F shows the proportion of SNVs within the cII gene for individually picked mutant plaques produced from BigBlue® mouse tissue and Duplex Sequencing of the gDNA of cII from the BigBlue® mouse tissues. SNVs are designated with pyrimidine as the reference. Duplex Sequencing yields the same spectrum of mutation from each treatment group as achieved by manual collection of 3,510 plaques (all three p-values>0.999 with chi-squared test). Proportions were calculated by dividing the total observations of SNVs by observed counts of reference bases within the cII interval and normalizing to one.

FIG. 3G shows the distribution of all mutations identified by direct Duplex Sequencing of cII across all BigBlue® tissue types and treatment groups by codon position and functional consequence. FIG. 3H shows distribution data for mutations identified among individually collected mutant plaques. With reference to FIGS. 3G and 3H together, direct Duplex Sequencing (FIG. 3G) identifies mutations along the entire gene causing all effect classes, whereas mutations from picked mutant plaques (FIG. 3H) are devoid of synonymous variants and mutations at the non-critical C- and N-termini of the protein. Without being bound by theory, it is believed that synonymous variants and mutations at the non-critical C- and N-termini of the protein does not cause disruption of gene function, which is necessary for selective growth and scoring within the plaque assay.

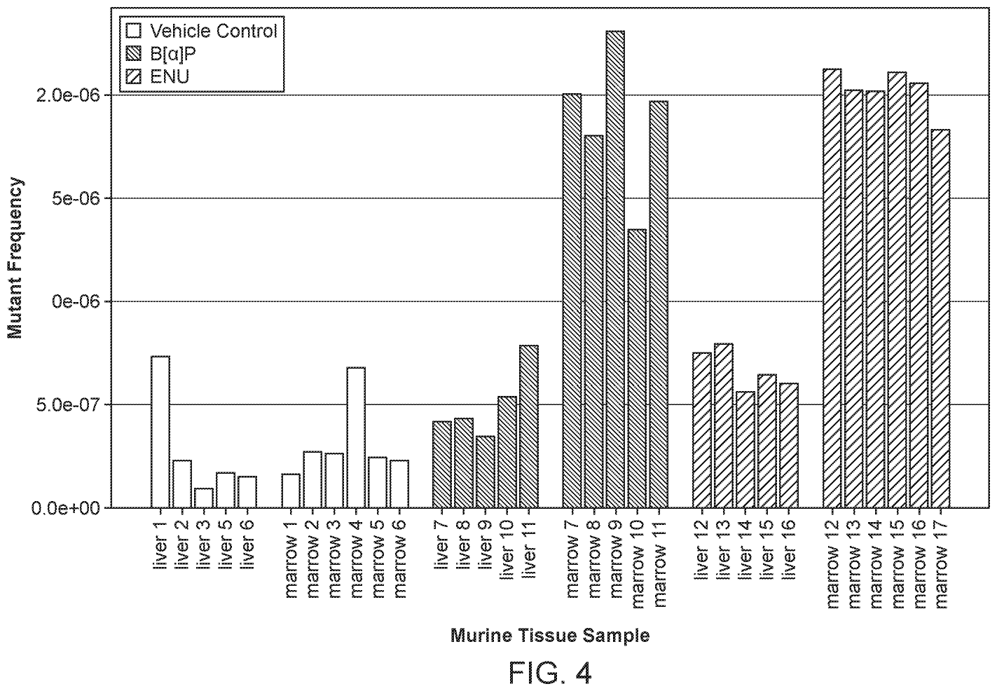

FIG. 4 is a bar graph showing MF measured by Duplex Sequencing is consistent within each treatment group. The MF, aggregated across all genes, was measured in liver and bone marrow by Duplex Sequencing. The number of unique mutants was low in vehicle control animals (1-13 mutations/1.4 billion base pairs) relative to mutagen-exposed mice (up to 118 mutation/2.6 billion base pairs). MF between animals within a group were reproducible in all treatment conditions and the low number of mutations in control animals (1 to 13) emphasizes the need for deep sequencing to generate robust estimates of MF.

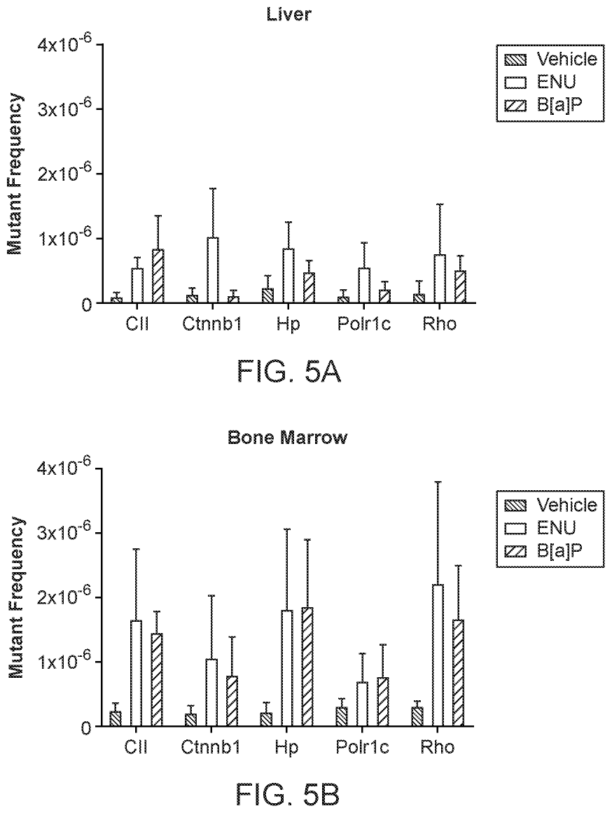

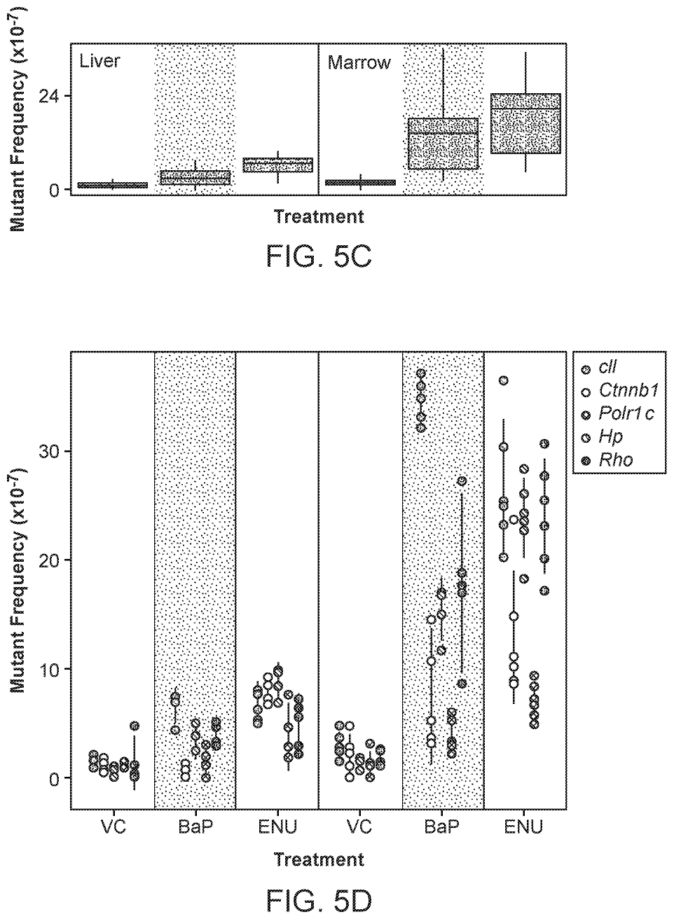

FIGS. 5A and 5B are bar graphs showing MF of endogenous genes as compared to cII transgene in liver (FIG. 5A) and bone marrow (FIG. 5B) and as measured by Duplex Sequencing. Each gene (˜3 to 6 kb) was sequenced at a depth of approximately 5000×, with the cII gene (˜350 bp×80 copies per genome) sequenced at a depth of ˜100K to 300K. The mutant frequency was calculated as describe above and with respect to FIGS. 3A-3D. As shown, endogenous genes exhibit a similar increase in MF as the cII transgene. Duplex Sequencing demonstrates that MF is higher in bone marrow than liver. Without being bound by theory, the higher rate of cell division in bone marrow may explain the higher MF levels detected for both tested mutagens. Furthermore the differences in response of endogenous genes shown in FIGS. 5A and 5B may relate to differences in transcriptional state or chromatic structure of the endogenous genes.

FIG. 5C is a box plot graph showing SNV MF calculated for Duplex Sequencing by genic regions for Liver and Bone Marrow, and FIG. 5D is a scatter plot showing individual measurements of aggregate data shown in FIG. 5C. Scatter points show individual measurements with 95% CI surrounding them. The box plot in FIG. 5C shows all four quartiles of all data points for that tissue and treatment category. Y-axis scales are presented linearly and in the 10⁻⁷ magnitude. Referring to FIG. 5C, the box plot summarizes the aggregate of the SNV mutation frequencies in the liver and bone marrow tissues across the four endogenous genes and the cII transgene of the Big Blue® mouse model shown in FIG. 5D. The extent of mutation induction is influenced by specific mutagen, tissue type and genetic locus.

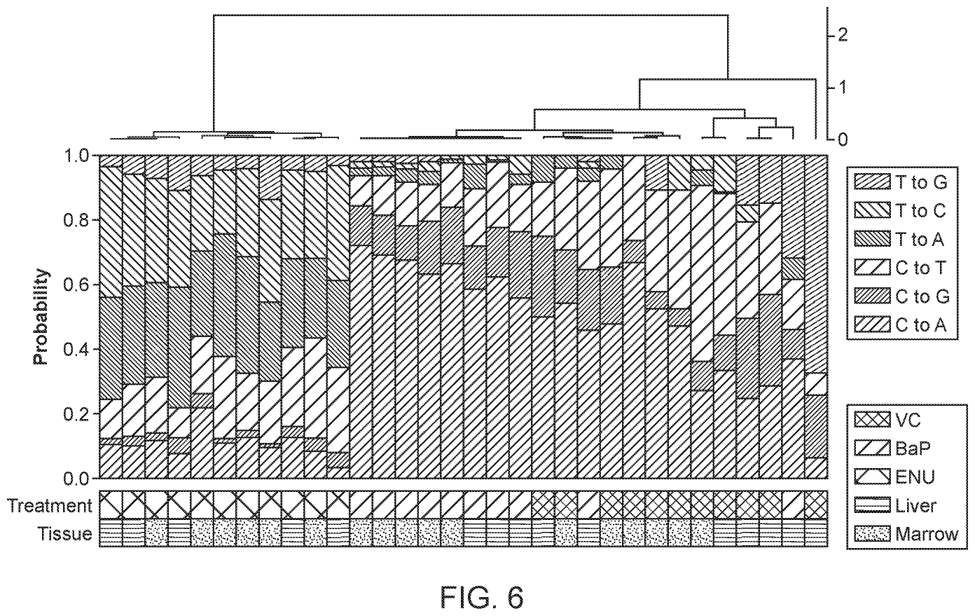

FIG. 6 is a bar graph showing the mutation spectrum of each test mutagen (e.g., treatment) within the tested tissues as measured by Duplex Sequencing. Referring to FIG. 6, the portion of each mutation, aggregated across all genes, and calculated for each sample and grouped by unsupervised hierarchical cluster analysis demonstrates that the mutation spectrum is unique to each treatment (e.g., test mutagen). Unsupervised cluster analysis of coded data permitted grouping of data based on mutation spectrum and demonstrates that ENU samples are easily identified in all tissues by a preponderance of T→C, T→A, and C→T mutations. Likewise, B[a]P samples are distinguished by C→A and G→T mutations.

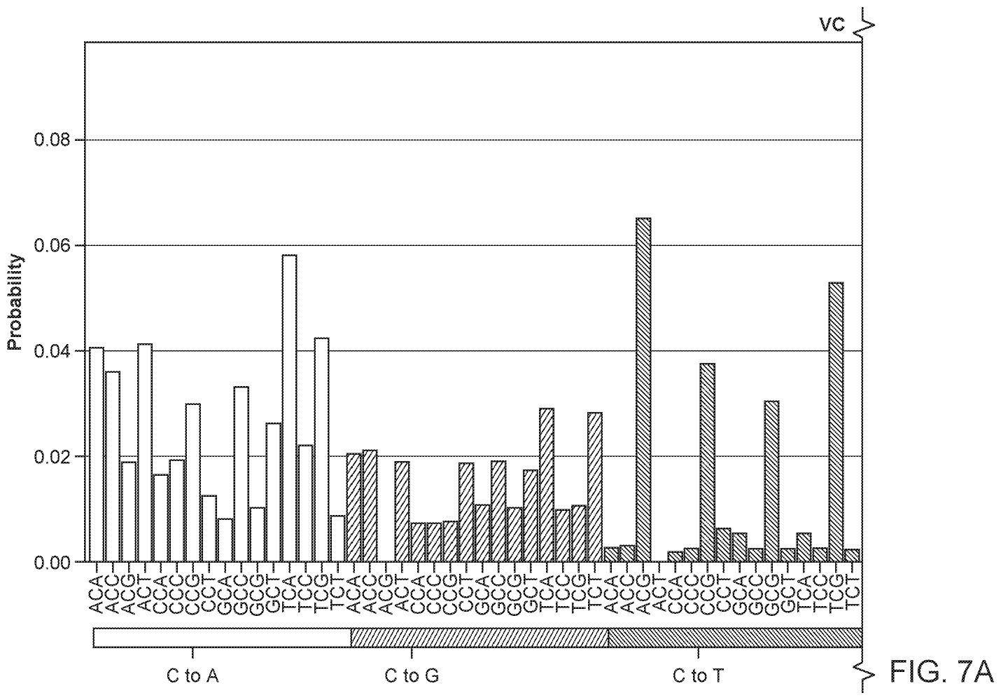

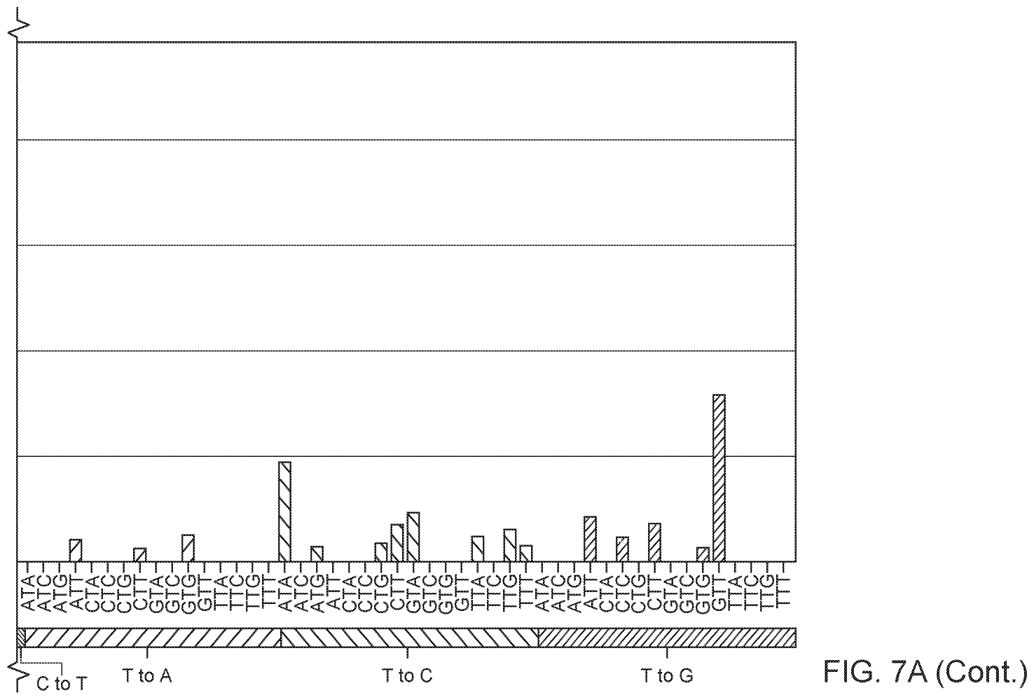

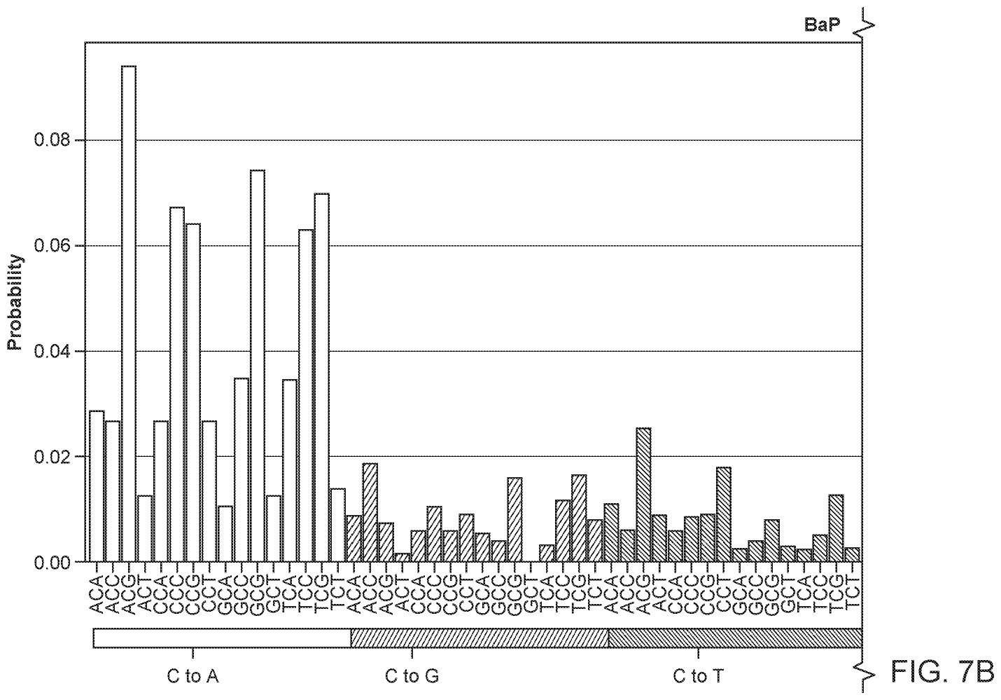

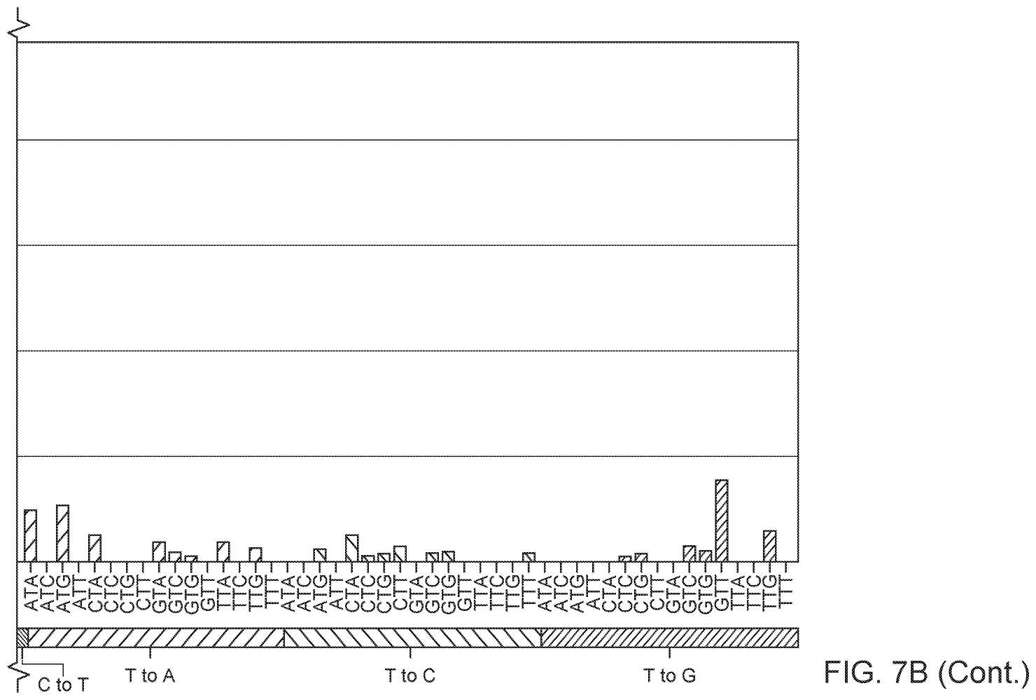

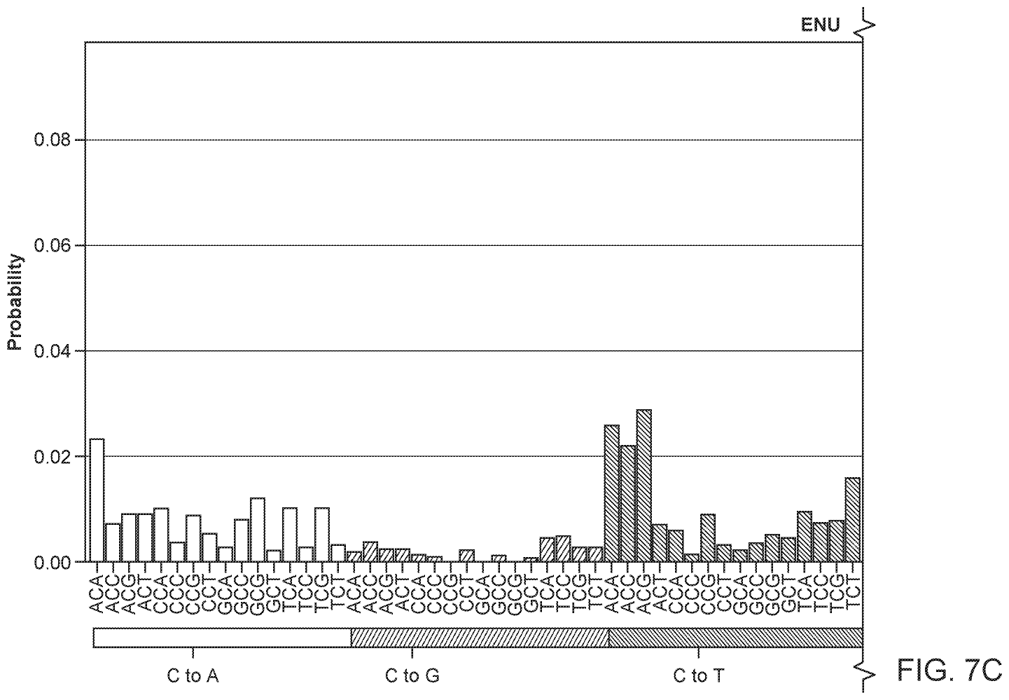

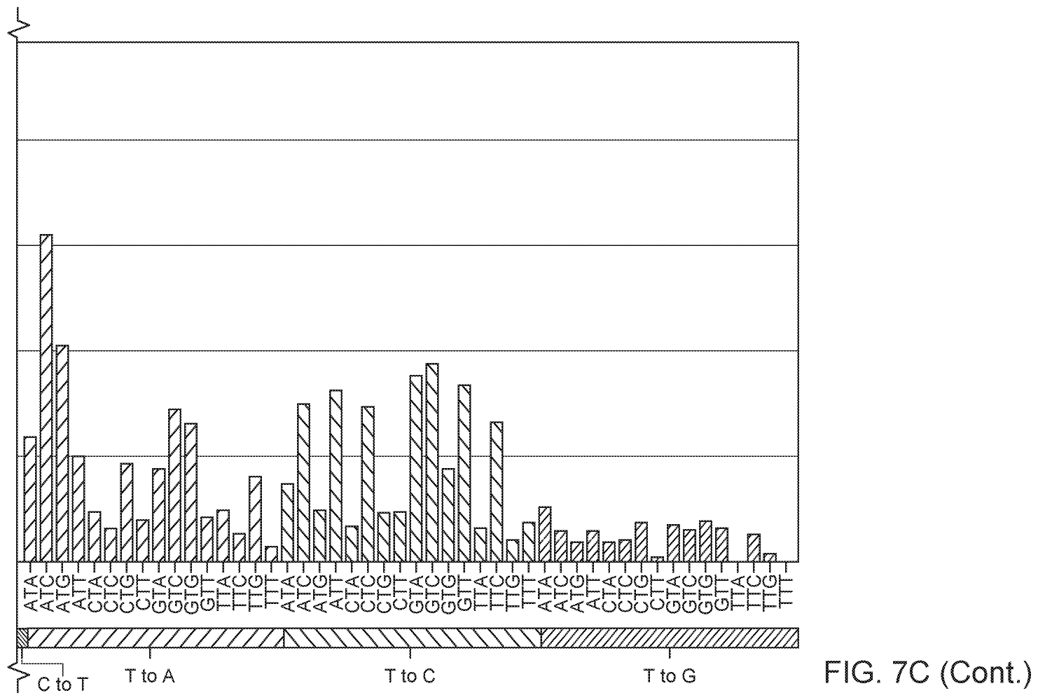

FIGS. 7A-7C are graphs showing mutation spectra in the context of adjacent nucleotide (i.e., trinucleotide spectra) for vehicle control (7A), B[a]P (7B), and ENU (7C). Mutational signature in trinucleotide spectra format provide information regarding different mechanism of mutagenesis and/or demonstrate mutational patterns unique for specific mutagens. For example, CCG and CGC contexts appear to be more vulnerable to the tobacco-associated carcinogen, B[a]P, than other contexts (FIG. 7B). This signature pattern may be similar to signature patterns demonstrated by aflatoxin exposure (e.g., may be a similar mechanism of mutagenesis). FIG. 7C illustrates that the alkylator, ENU, has two vulnerable contexts that match the IUPAC code GTS where S+[G][C], and is a heavy inducer of transition mutations.

In this example, it has been demonstrated that mutation load in ENU and B[a]P-treated bone marrow and liver samples was significantly increased relative to controls, comparable to traditional BigBlue® cII mutant plaque frequency (mutant frequency MF), and varied similarly by tissue type. Spectrum evaluation revealed distinctive patterns of INDELS and single base substitutions in each treatment group. trinucleotide base analysis demonstrated that adjacent nucleotide context strongly modulates mutagenic potential; the most extreme hotspots were CCG and CGC for B[a]P and GTG and GTC for ENU. Duplex Sequencing was extended to 4 endogenous genes: Polr1c, rhodopsin, haptoglobin, and beta-catenin. Again, MF increased in animals exposed to ENU and B[a]P, but varied significantly by genomic locus, likely reflecting transcriptional status. In this example, Duplex Sequencing demonstrates to be a successful method for detecting mutations in the cII transgene, an accepted pre-clinical safety biomarker in TGR assays, but further, this example demonstrates that Duplex Sequencing can be the basis of risk assessment tools based on endogenous cancer-related genes.

### Example 2

Direct quantification of in vivo chemical mutagenesis in mammalian genomes using duplex sequencing. This section describes an example wherein Duplex Sequencing is used to determine if early mutations in cancer driver genes reflect tumorigenic potential of test mutagens.

In this example, the impact of a urethane is examined in different mouse tissue types (lung, spleen, blood) in an FDA-approved cancer-predisposed mouse model: Tg.rasH2 (Saitoh et al. Oncogene 1990. PMID 2202951). This mouse contains ˜3 tandem copies of human Hras with an activating enhancer mutation to boost expression on one hemizygous allele. These mice are predisposed to splenic angiosarcomas and lung adenocarcinomas, and are routinely used for 6 month carcinogenicity studies to substitute for 2 year native animal studies. Tumors found in the mice have usually acquired activating mutations in one copy of the human Hras protooncogene. In this addition to the 4 native mouse genes (Rho, Hp, Ctnnb1, Polr1c), the native mouse Hras and human Hras transgene are also analyzed in this example.

In this example, Tg.rasH2 mice (n=5/group) were dosed with vehicle or a carcinogenic dose of urethane (day 1,3,5) and sacrificed on day 29 for mutation detection by Duplex Sequencing in target tissues (lung, spleen) and whole blood. The endogenous genes (Rho, Hp, Ctnnb1, Polr1c) and the native mouse and human Hras (trans)genes were also sequenced.

Tumors (splenic hemangiosarcomas; lung adenocarcinoma) were collected at week 11 from animals (n=5/group) dosed with urethane and subjected to whole exome sequencing (WES) to identify characteristic cancer driver mutations (CDM) in these tumors.

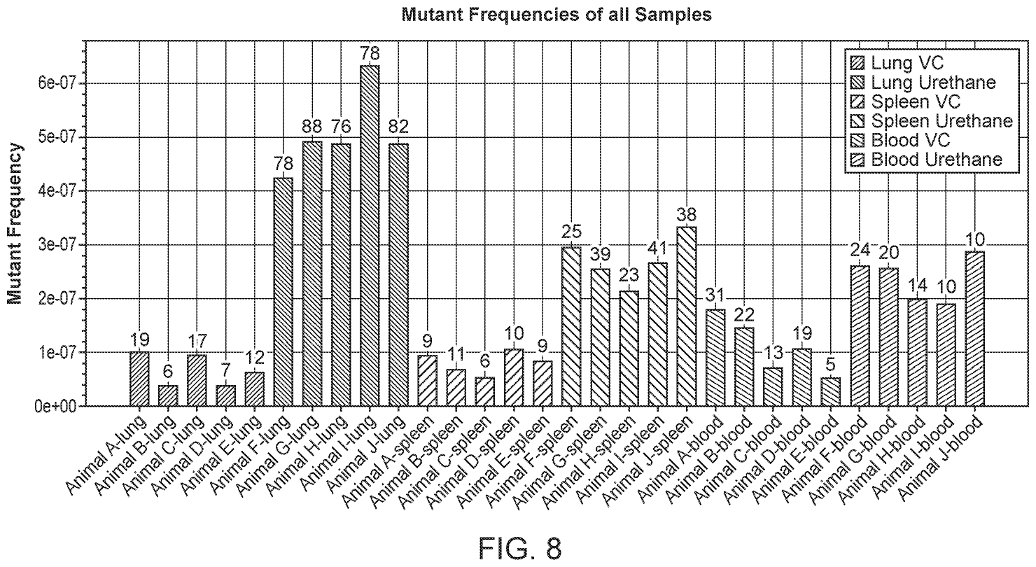

FIG. 8 is a bar graph showing mutant frequency (MF) of lung, spleen and blood samples for control and experimental animals subjected to urethane. In this analysis, every unique variant detected was counted as one mutation, which were summed per sample. This was divided by the total number of Duplex Bases sequenced and across the entire capture region. The number of events is noted above each sample. In total, across all 30 samples, 3,966,947,832 Duplex Sequenced base pairs were generated. As shown in FIG. 8, the mutation induction is consistent between animals in the same treatment group and confidence increases with sequencing depth.

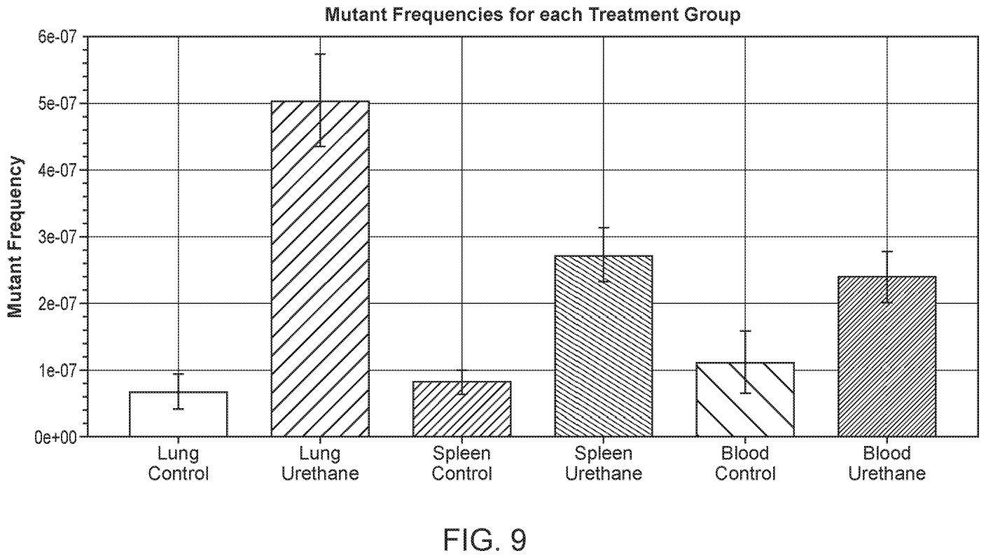

FIG. 9 is a bar graph showing the average minimum point mutant frequency across each group of tissue samples (error bars are +/− one standard deviation).

TABLE 1

| Tissue | Treatment | Mutation Frequency | Fold Increase | p-value |
|---|---|---|---|---|
| Lung | Vehicle Control | 0.67e−07 | | |
| Lung | Urethane | 5.04e−07 | 7.5x | 6.73e−05 |
| Spleen | Vehicle Control | 0.83e−07 | | |
| Spleen | Urethane | 2.73e−07 | 3.3x | 1.92e−04 |
| Blood | Vehicle Control | 1.11e−07 | | |
| Blood | Urethane | 2.39e−07 | 2.2x | 0.003025 |

Referring to FIG. 9 and Table 1 together, differences between vehicle control (VC) and treatment groups were highly significant. A Welch's t-test (for unequal variances) was used to determine the significance of the mutagen treated tissue's mutant frequency over that of the control for that tissue. The slightly wider confidence intervals with blood reflects a lower average depth of sequencing in the blood VC samples in this particular example. It is anticipated that this can be corrected using the methods described herein.

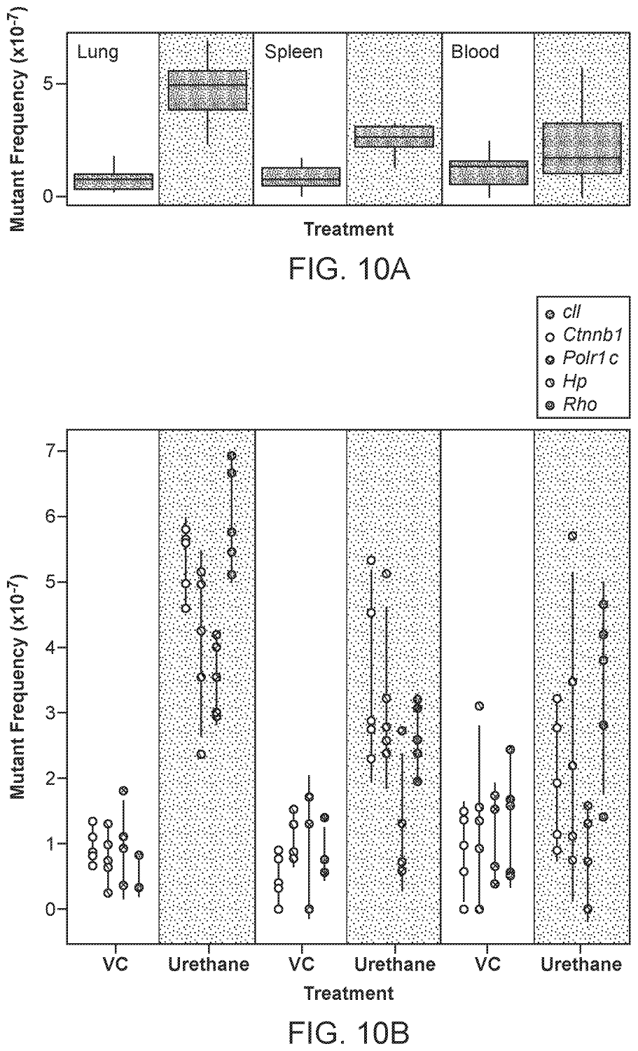

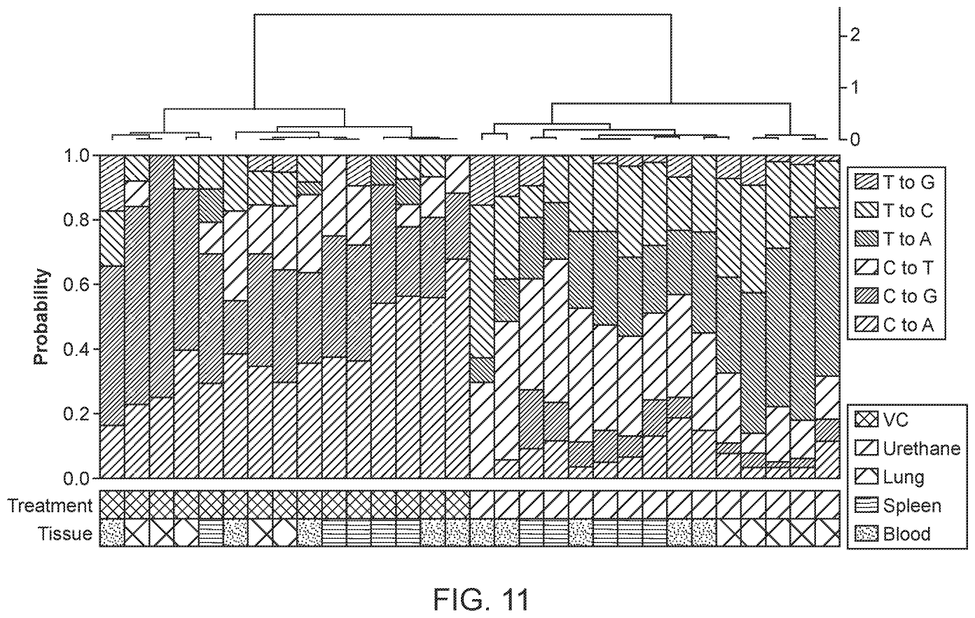

FIG. 10A is a box plot graph showing SNV MF calculated for Duplex Sequencing by genic regions for Lung, Spleen and Blood for the indicated treatments categories, and FIG. 10B is a scatter plot showing individual measurements of aggregate data shown in FIG. 10A. Scatter points show individual measurements with 95% CI surrounding them. The box plot in FIG. 10A shows all four quartiles of all data points for that tissue and treatment category. Y-axis scales are presented linearly and in the 10⁻⁷ magnitude. Referring to FIG. 10A, the box plot summarizes the aggregate of the SNV mutation frequencies in the lung, spleen, and blood of the Tg-rasH2 mouse model shown in FIG. 10B. There is no cII transgene in the Tg-rasH2 mouse model. The extent of mutation induction is influenced by specific mutagen, tissue type and genetic locus. FIG. 11 is a bar graph showing the mutation spectrum of urethane and VC within the tested tissues as measured by Duplex Sequencing. Referring to FIG. 11, unsupervised cluster analysis of coded data permitted grouping of data based on mutation spectrum. This data demonstrates that simple spectrum of nucleotide variation alone can identify exposure. In other words, if the mutagen was unknown, such mutagen could be identified de novo by via Duplex Sequencing of DNA of an exposed organism by nature of the mutation spectrum.

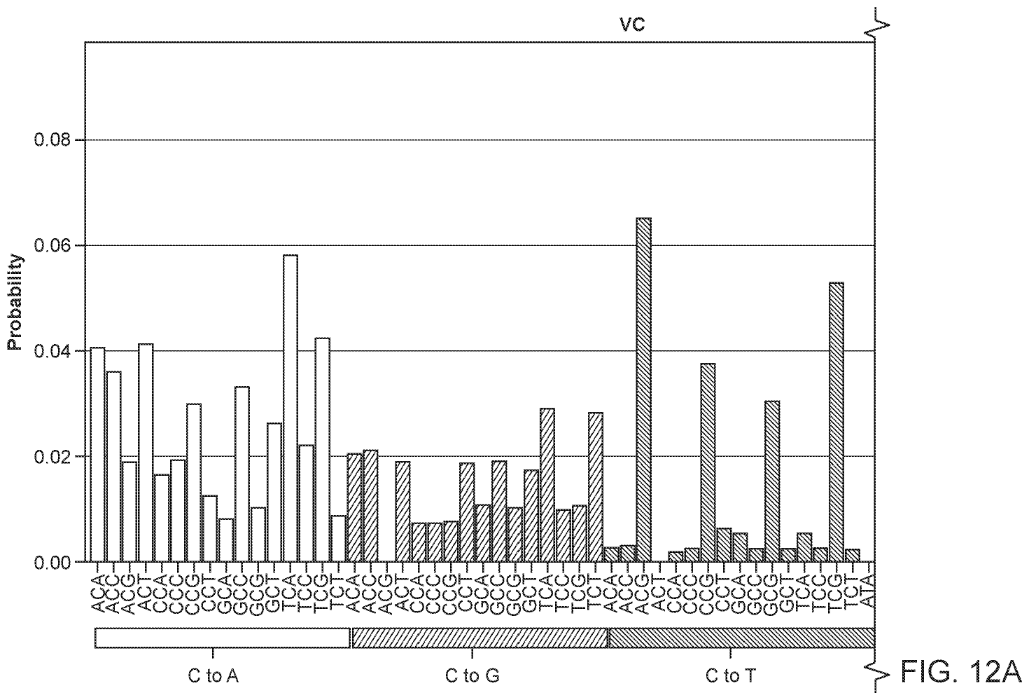

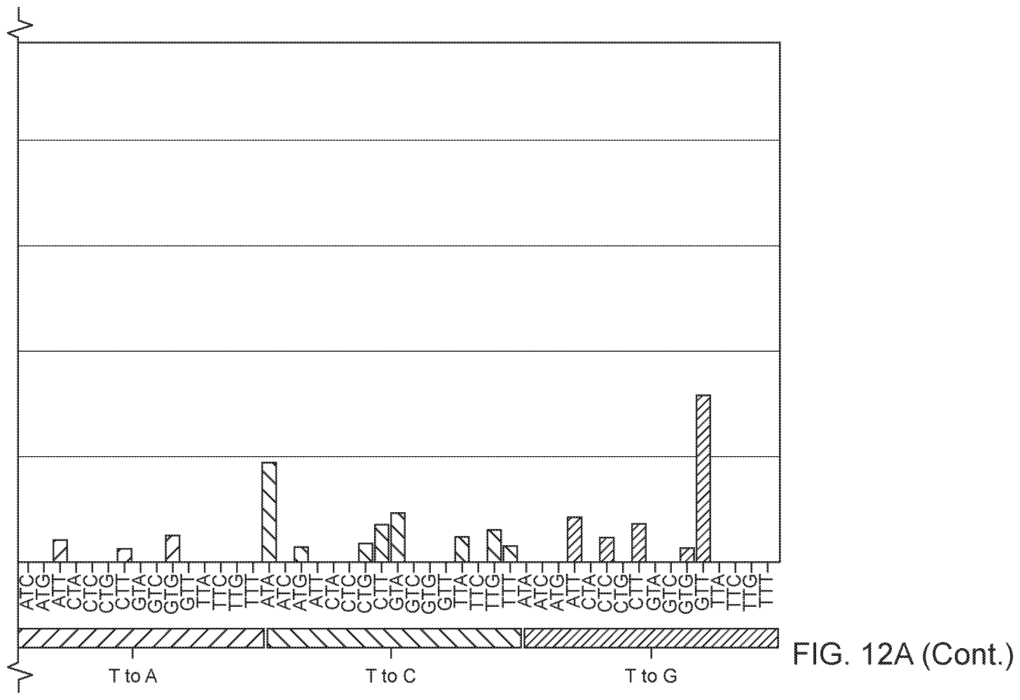

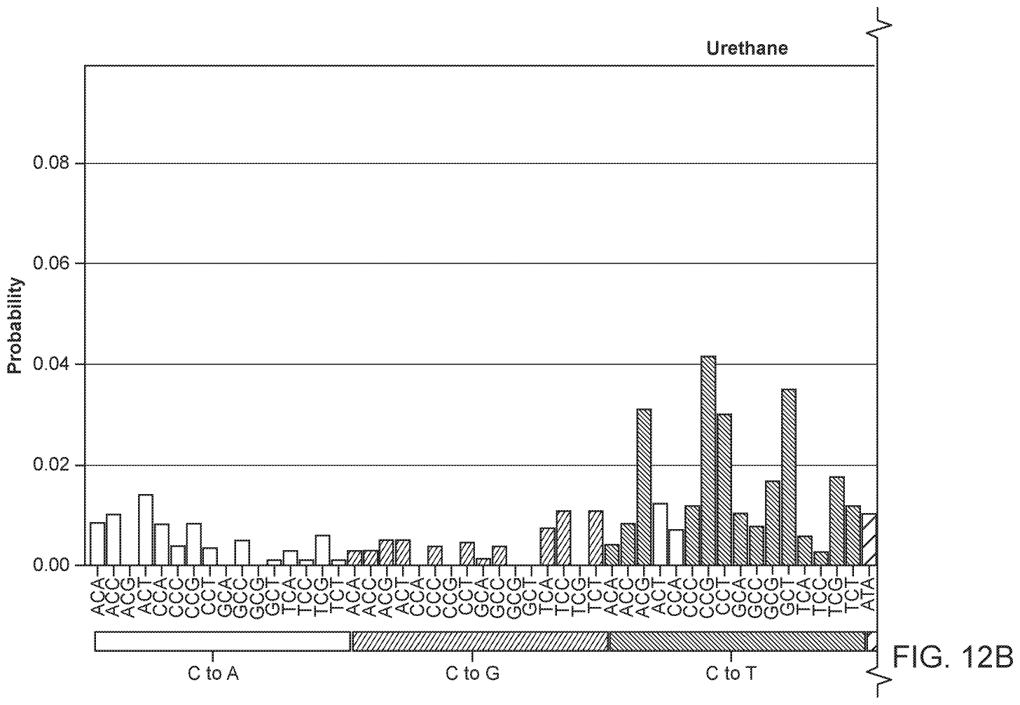

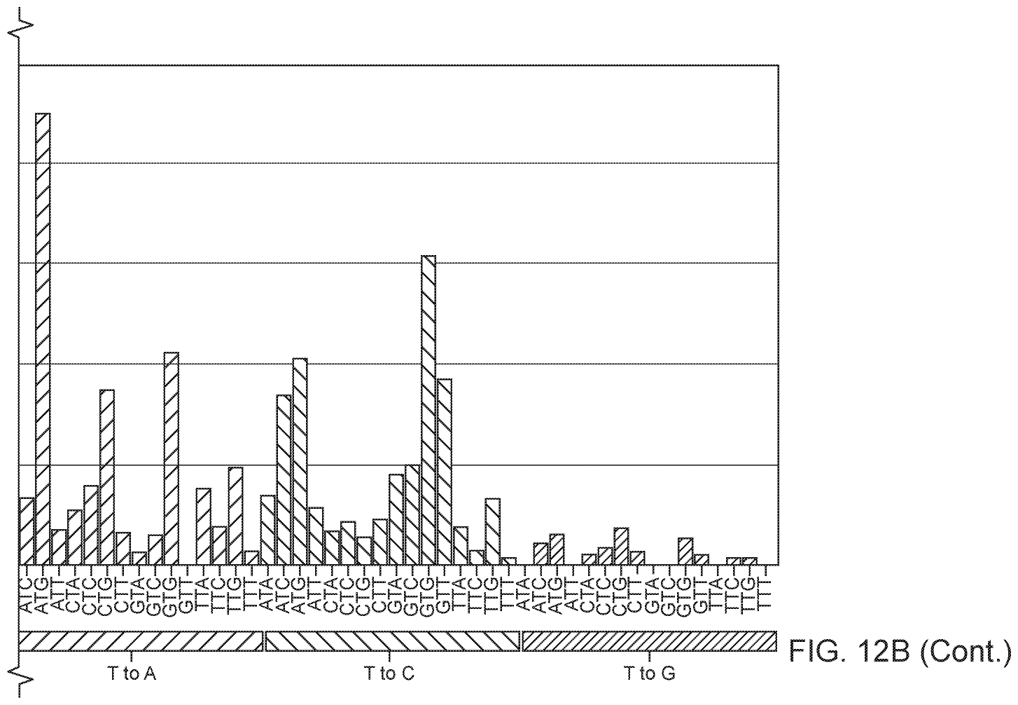

FIGS. 12A and 12B are graphs showing mutation spectra in the context of adjacent nucleotides (i.e., trinucleotide spectra) for vehicle control (12A), and urethane (12B). Mutational signature in trinucleotide spectra format provide information regarding different mechanisms of mutagenesis and/or demonstrate mutational patterns unique for specific mutagens. Accordingly, the detailed breakdown of each mutation class within its trinucleotide context (“triplet signature”) reveals a highly unique fingerprint for each treatment group, consistent with known signatures of clonal mutations from tumors caused by such exposures. In untreated animals C:G→A:T and C:G→G:C mutations caused, respectively by oxidation of guanine and deamination of cytosine and 5-me-cytosine, which is a known pattern from aging, were detected. Following urethane treatment, T:A→A:T within the motif “NTG” is shown as the most common mutation.

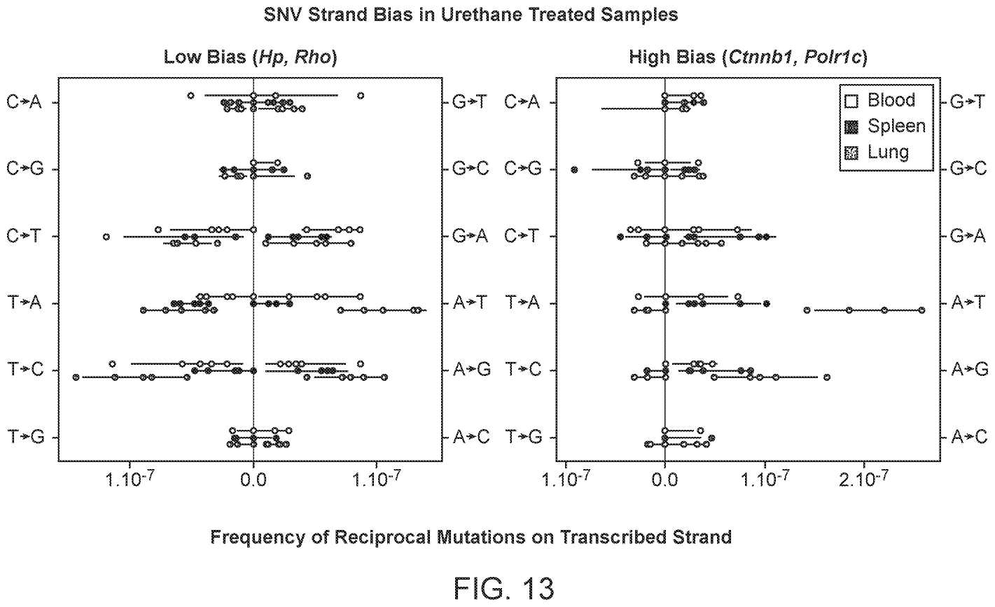

FIG. 13 shows that single nucleotide variant (SNV) strand bias was observed in Ctnnb1 and Polr1c but not in Hp or Rho genomic regions. SNV notation are normalized to the reference nucleotide in the forward direction of the transcribed strand. Individual replicates are shown with points and 95% confidence intervals, with line segments. All mutation frequencies were corrected for the nucleotide counts of each reference base within the variant calling region. The null hypothesis for no strand bias is equal frequencies for reciprocal mutations. The bias is evident in Ctnnb1 and Polr1c as C>N and T>N variants are at uniform frequencies and G>N and A>N variants are at elevated frequencies. Compared to Hp and Rho, and without being bound by theory, it is believed that this difference is due to transcription-coupled nucleotide excision repair and the relative expression levels of these genes.

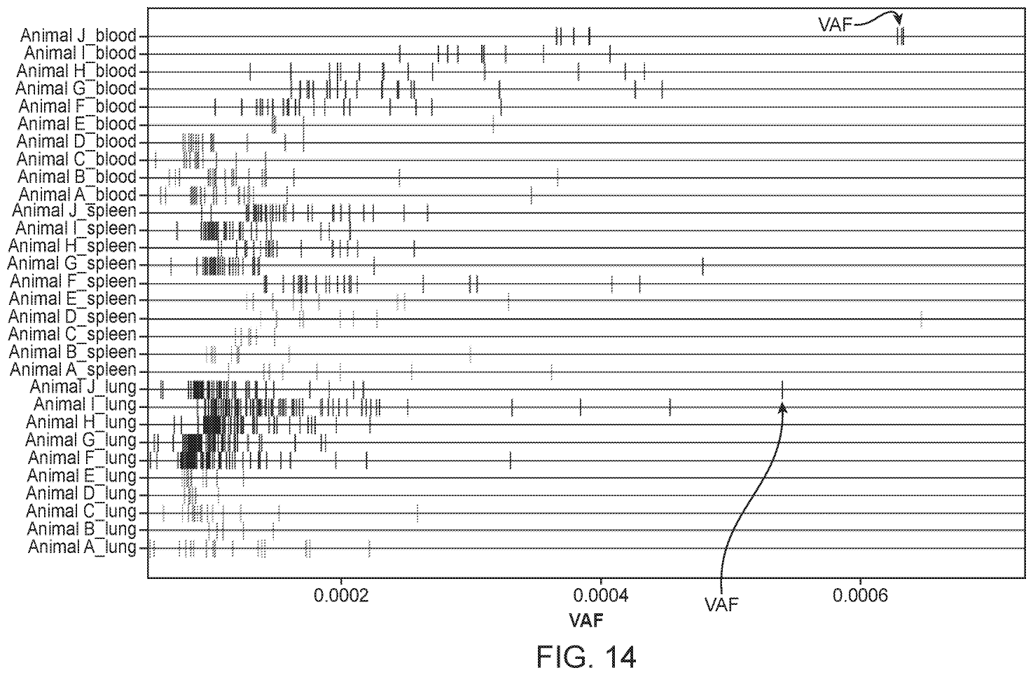

FIG. 14 is a graph illustrating early stage neoplastic clonal selection of variant allele fractions as detected by Duplex Sequencing. The vast majority of mutations identified occurred in single molecules and at very low variant allele fractions (VAFs), e.g., on the order of 1/10,000. A few variants were found in multiple molecules in a sample and were identified as having considerably higher VAFs.

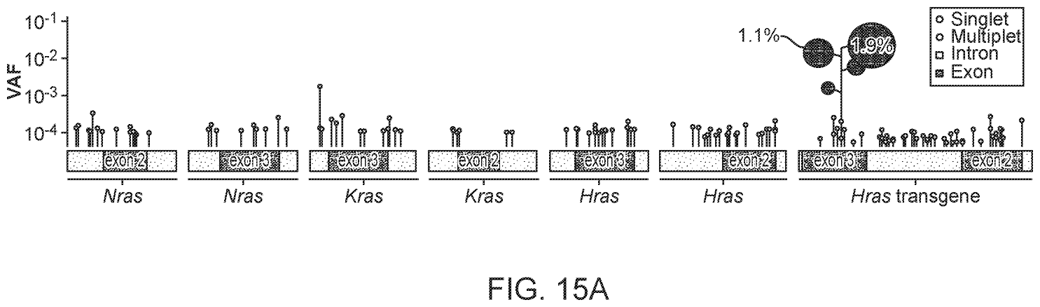

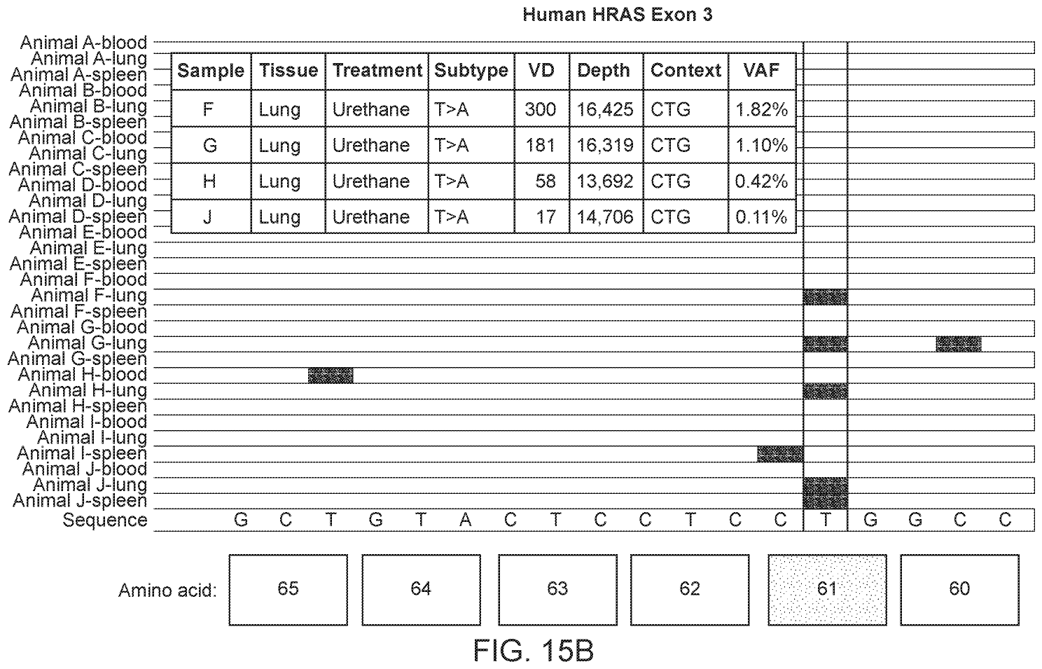

FIG. 15A is a graph illustrating SNVs plotted over the genomic intervals for the exons captured from the Ras family of genes, including the human transgenic loci, in the Tg-rasH2 mouse model. Singlets are mutations found in a single molecule. Multiplets are an identical mutation identified within multiple molecules within the same sampler and may represent a clonal expansion event. The height of each point corresponds to the variant allele frequency (VAF) of each SNV, with the with the size of the point corresponds to the for multiplet observations only. The location and relative frequency of Ras family human cancer mutational hotspots in COSMIC are indicated below each gene. FIG. 15B is a graph illustrating single nucleotide variants (SNVs) aligning to exon 3 of the human HRAS transgene. Highlighted is the center residue in codon number 61 in exon 3 of human HRAS, the most common HRAS cancer-driving hotspot.

Referring to FIGS. 15A and 15B together, a cluster of T>A transversions were observed in ⅘ urethane-treated lung samples and ⅕ urethane-treated splenic samples at the human oncogenic Hras codon 61 hotspot. In particular, four out of five treated lung samples harbored this mutation at variant allele frequencies of 0.1%-1.8%. Notably these clones are of the transversion T>A in the context NTG, which is characteristic of urethane mutagenesis (referring to strong favoring of NTG sites on FIG. 12B). In addition, two treated spleen samples had mutations in this codon: one at this same position and one on an adjoining base pair. The observation that ⅘ treated lung samples had clonally expanded pathogenic mutations by day 29, whereas very few mutations seen elsewhere on the panel were seen as >1 member clones or were seen repeated in multiple samples (as high VAF multiplets in a well-established cancer driver) is a strong indication of positive selection soon after exposure. Furthermore, Duplex Sequencing methods, in accordance with embodiments of the present technology, provides the necessary sensitivity to detect such early stage neoplastic clonal selection.

TABLE 2

| Mutation | Number of | | |
|---|---|---|---|
| count | families | | |
| 1 | 829 | | |
| 2 | 8 | | |
| 4 | 1 | | |
| 17 | 1 | | Oncogenic AA 61 T > |
| 58 | 1 | | A mutations in Human |
| 181 | 1 | {close oversize brace} | HRAS gene in urethane |
| 300 | 1 | | treated lung tissue |

Referring to Table 2, 97.5% of mutations were identified in a single molecule only, 1% were seen in two molecules and about 0.5% were seen in >2 molecules. The four highest level clones all occurred with oncogenic mutation in AA 61, the recurrent tumor hotspot in human HRAS. That the highest level clones also appear at cancer hotspots further emphasized the magnitude of the strong selective pressure.

A far larger amount of DNA was extracted per sample than was converted into sequenced Duplex Molecules. The portion of tissue samples extracted yielded roughly 5 μg of genomic DNA. Converting this into genome equivalents, and multiplying by three yields the number of tg.HRAS copies in the extraction. Only ˜⅓% of this was sequenced so roughly 300 times more mutants were present in the original portion of tissue sampled than detected.

TABLE 3

| | | | Copies | Depth at | % copies | | Mutant cells in |
|---|---|---|---|---|---|---|---|
| Sample | ng DNA | Genomes | tg.HRAS | AA 61 | sequenced | Mutants | original sample |
| 9957-Lung 1 | 5,640 | 1,692,000 | 5,076,000 | 16,425 | 0.324% | 300 | 92,712 |
| 9958-Lung 1 | 4,400 | 1,320,000 | 3,960,000 | 16,319 | 0.412% | 181 | 43,922 |
| 9959-Lung 1 | 4,480 | 1,344,000 | 4,032,000 | 13,692 | 0.340% | 58 | 17,080 |
| 9961-Lung 1 | 4,700 | 1,410,000 | 4,230,000 | 14,706 | 0.348% | 17 | 4,890 |

In this example, the selected clones encompassed more than 90,000 cells in the highest allele fraction clone. As a result, by calculation, within the 29 days of the study, e.g., from the time of mutation exposure, and assuming no cell death, the doubling time of these cells was roughly every 1.8 days 2\hat{ }(29/1.8)˜90,000. Without being bound by theory, this calculated rate of cell doubling suggests the likely ability to detect these selected mutations in a short time frame (e.g., as few as two weeks).

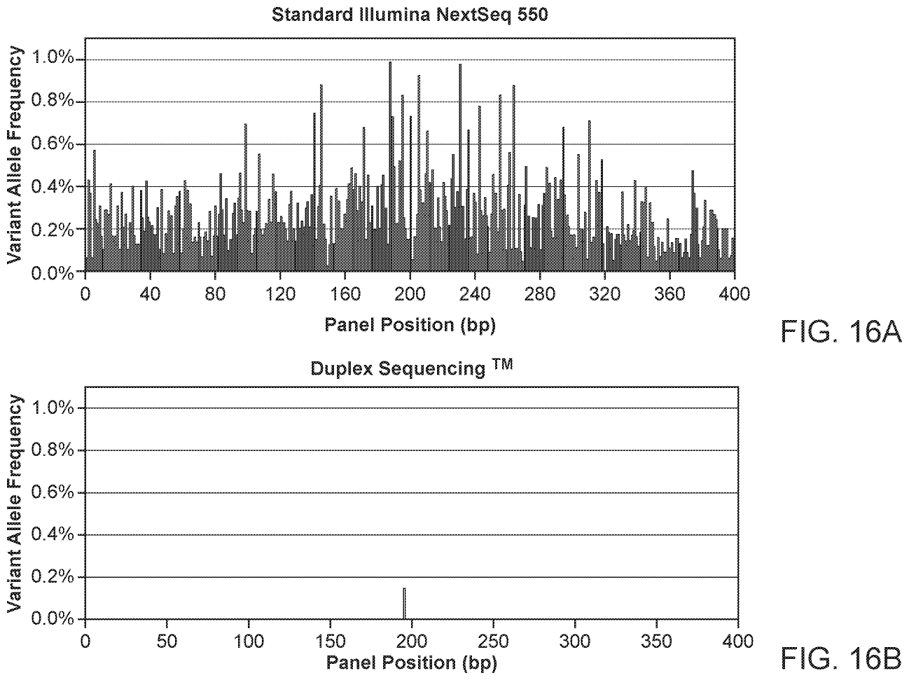

FIGS. 16A-16B are graphical representations of sequencing data from a representative 400 base pair section of human HRAS in mouse lung following urethane treatment using conventional DNA sequencing (FIG. 16A) and Duplex Sequencing (FIG. 16B). Conventional DNA sequencing has an error rate of between 0.1% and 1%, which obscures the presence of genuine low frequency mutations. FIG. 16A shows conventional sequencing data from a representative 400 BP section of one gene (human HRAS) of one sample (mouse lung) in the present study. Each bar corresponds to a nucleotide position. The height of each bar corresponds to the allele fraction of non-reference bases at that position when sequenced to >100,000× depth. Every position appears to be mutated at some frequency; nearly all of these are errors. Referring to FIG. 16B, when processed with Duplex Sequencing, it becomes apparent that only one mutation is authentic.

The results of the experimental analysis of this example demonstrates that Duplex Sequencing quantifies induction of mutations by urethane extremely robustly and with tight replicate confidence intervals. Further, the extent of mutation induction is tissue-specific, with lung being more prone than spleen and blood. The simple mutational spectrum of urethane exposure is clean and unbiased clustering can discriminate between groups. The triplet mutation spectrum of urethane shows a strong propensity for T→A and T→C mutations within the context of “NTG” and the mutation spectrum is distinguishable from the vehicle control (and other mutagens; see example 1).

Additionally, mutation induction in peripheral blood closely mirrored that seen in the spleen and suggests that in-life sampling of peripheral blood could, for some mutagens, substitute for necropsy (or biopsy). Furthermore, this example demonstrated that even at day 29 clear evidence of selection for oncogenic mutations in the human HRAS transgene is demonstrated using Duplex Sequencing. The spectrum of mutation at this hotspot accurately reflected the effects of this known mutagen. Hence, Duplex Sequencing can provide early and accurate data with respect to evaluating early cancer driver mutations as biomarker of future cancer risk. Cross-species contamination persisted at extremely low levels but removal of foreign species contamination was performed automatically and confidently.

### Example 3

Analysis of mutagen signatures in mammalian genomes using Duplex Sequencing. This section describes an example wherein data generated from Duplex Sequencing analysis can be used to generate and compare mutagenic signatures for the identification mutagens and/or to identify a mutagen exposure.

The Catalogue of Somatic Mutations In Cancer (COSMIC) database provides reference to “mutational signatures”, defined as the unique combination of mutation types found present in the genome. Somatic mutations that are present in all cells of the human body and occur throughout life. Such somatic mutations are the consequence of, for example, multiple mutational processes, including the intrinsic slight infidelity of the DNA replication machinery, exogenous or endogenous mutagen exposures, enzymatic modification of DNA and defective DNA repair.

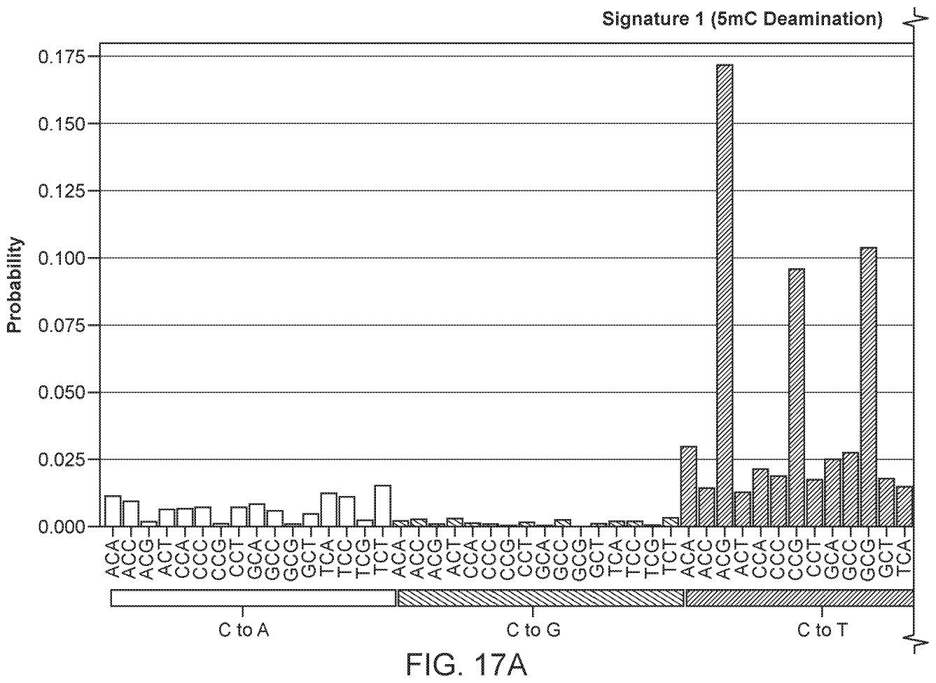

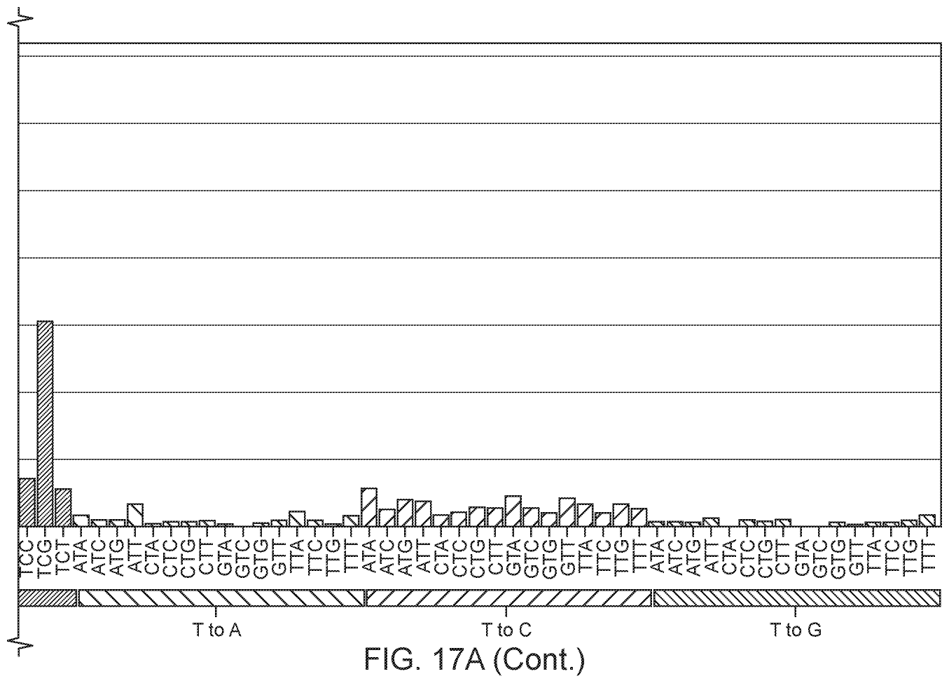

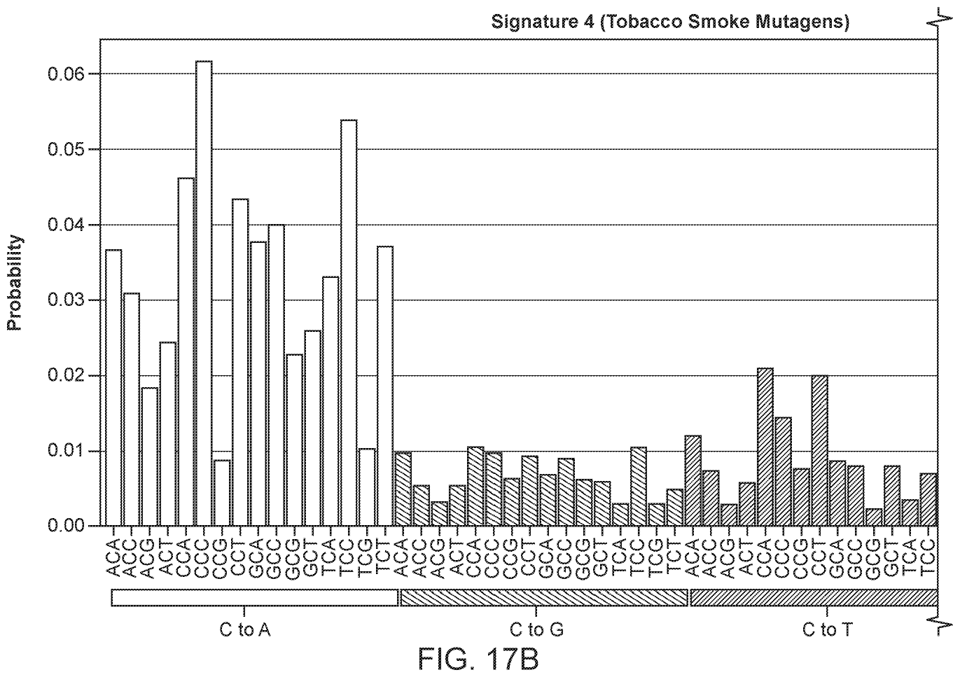

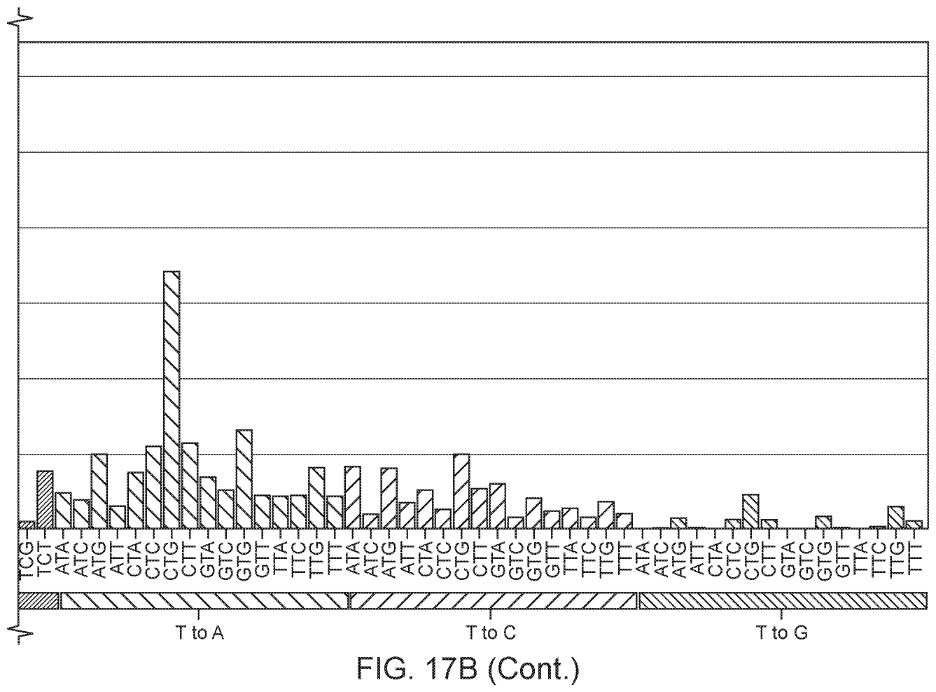

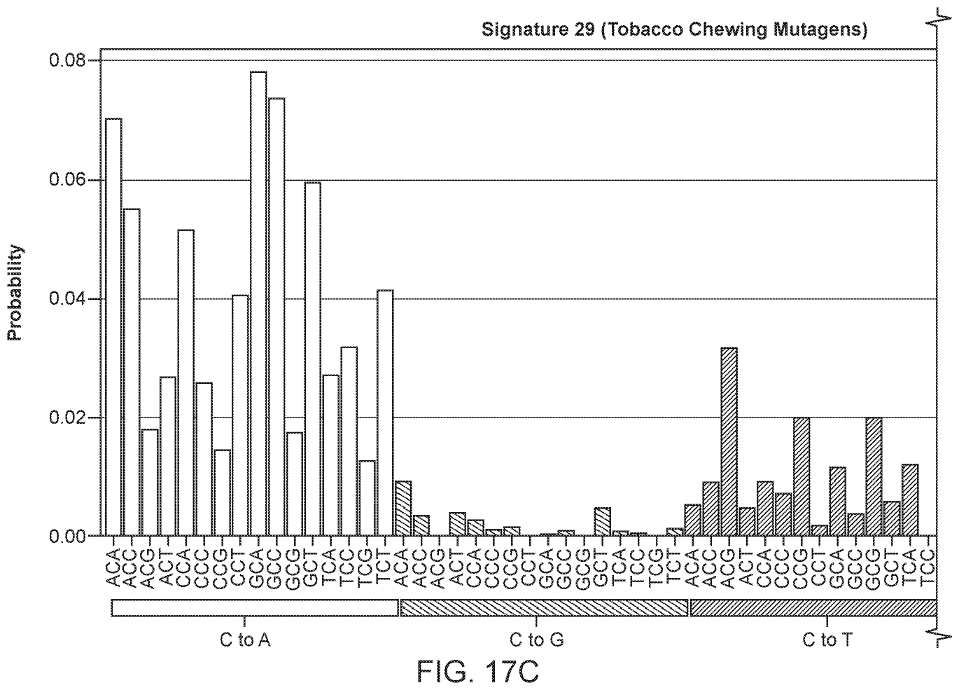

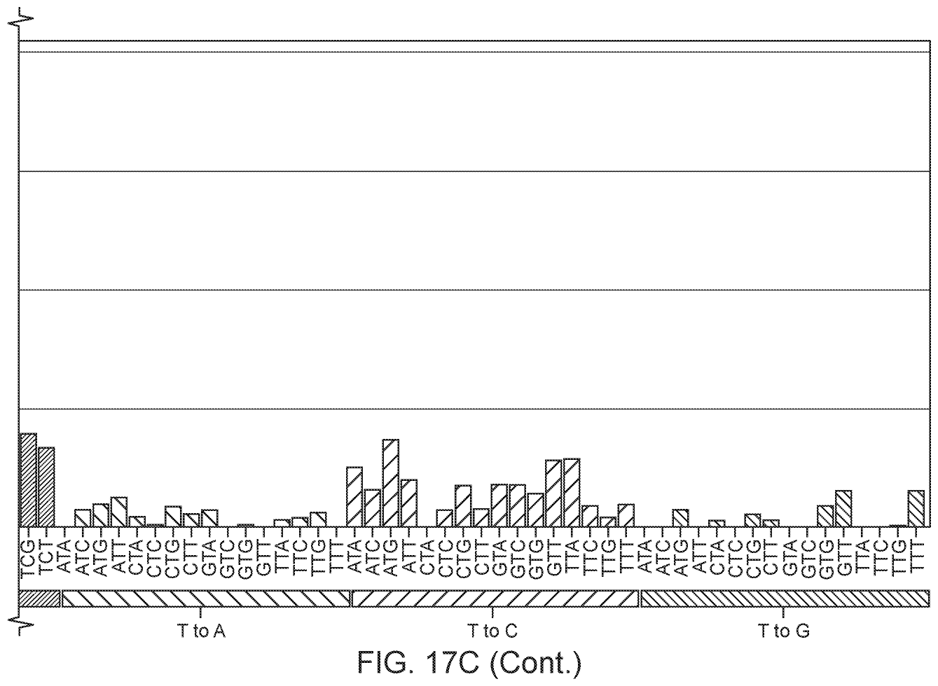

FIGS. 17A-17C are graphs showing mutation spectra in the context of adjacent nucleotides (i.e., trinucleotide spectra) for Signature 1 (FIG. 17A), Signature 4 (FIG. 17B), and Signature 29 (FIG. 17C) from COSMIC. Referring to FIG. 17A, signature 1 is seen in all cancer types with a proposed etiology of being caused by spontaneous deamination of 5-methyl-cytosine, resulting in C>T transitions at CpG sites. Referring to FIGS. 17B-17C, signatures 4 and 29 are correlated with smoking and are driven by a major mutagen in tobacco: benzo[a]pyrene. Although similar in pattern, signature 4 is most frequently observed in lung cancers in smokers whereas signature 29 is seen predominantly in squamous esophageal cancer, which is most frequent in smokers and users of chewing tobacco.

TABLE 4

| Feature | Example 1 | Example 2 | Total |
|---|---|---|---|
| Mouse Model | BigBlue | Tg-rasH2 | 2 strains |
| Tissues (samples) | Liver (15) | Lung (10) | 5 types |
| | Marrow (17) | Spleen (10) | |
| | | Blood (10) | |
| Mutagen (animals | B[a]P (10) | Urethane (15) | 3 mutagens |
| per group) | ENU (11) | VC (15) | |
| | VC (11) | | |
| Samples | 32 | 30 | 62 |
| Endogenous Loci | Polr1c | Polr1c | 7 native genes |
| | Rho | Rho | |
| | Ctnnb1 | Ctnnb1 | |
| | Hp | Hp | |
| | | Hras | |
| | | Kras | |
| | | Nras | |
| Transgenic Loci | cII | Human Hras | 2 transgenes |
| Duplex BP | 4,074,604,194 | 4,348,868,321 | 8,423,472,515 |

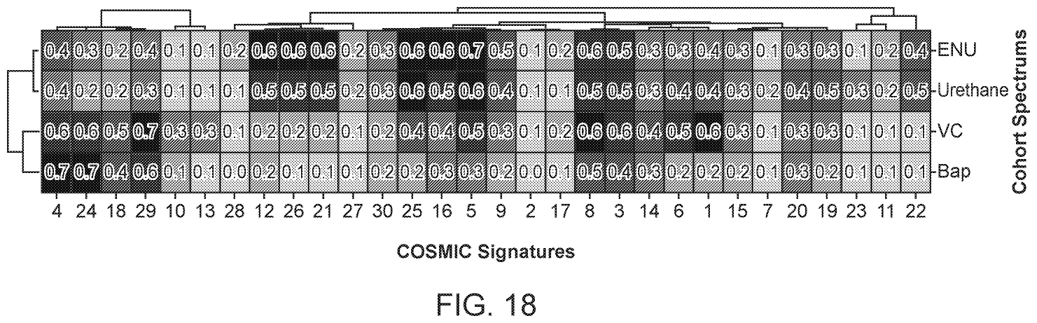

Table 4 provides experimental parameters and data derived from Examples 1 and 2 discussed herein. FIG. 18 shows unsupervised hierarchical clustering of all 30 published COSMIC signatures and the 4 cohort spectra from Examples 1 and 2. Clustering was performed with the weighted (WGMA) method and cosine similarity metric. Notably, benzo[a]pyrene (BaP) is very similar to both Signature 4 and 29 which have been correlated with BaP exposure through tobacco consumption or inhalation. Vehicle control (VC) is like Signature 1, a pattern linked to spontaneous deamination of 5-methyl-cytosine and is believed to represent a mixture of both the mutagenic effect of reactive oxidative species and spontaneous deamination of 5-methyl-cytosine.

This example demonstrates that Duplex Sequencing can be used to generate mutation spectra analysis that can be compared or referenced to known mutational signatures for purposes of identification and other analysis.

### Suitable Computing Environments

The following discussion provide a general description of a suitable computing environment in which aspects of the disclosure can be implemented. Although not required, aspects and embodiments of the disclosure will be described in the general context of computer-executable instructions, such as routines executed by a general-purpose computer, e.g., a server or personal computer. Those skilled in the relevant art will appreciate that the disclosure can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The disclosure can be embodied in a special purpose computer or data processor that is specifically programmed, configured or constructed to perform one or more of the computer-executable instructions explained in detail below. Indeed, the term “computer”, as used generally herein, refers to any of the above devices, as well as any data processor.

The disclosure can also be practiced in distributed computing environments, where tasks or modules are performed by remote processing devices, which are linked through a communications network, such as a Local Area Network (“LAN”), Wide Area Network (“WAN”) or the Internet. In a distributed computing environment, program modules or sub-routines may be located in both local and remote memory storage devices. Aspects of the disclosure described below may be stored or distributed on computer-readable media, including magnetic and optically readable and removable computer discs, stored as firmware in chips (e.g., EEPROM chips), as well as distributed electronically over the Internet or over other networks (including wireless networks). Those skilled in the relevant art will recognize that portions of the disclosure may reside on a server computer, while corresponding portions reside on a client computer. Data structures and transmission of data particular to aspects of the disclosure are also encompassed within the scope of the disclosure.

Embodiments of computers, such as a personal computer or workstation, can comprise one or more processors coupled to one or more user input devices and data storage devices. A computer can also coupled to at least one output device such as a display device and one or more optional additional output devices (e.g., printer, plotter, speakers, tactile or olfactory output devices, etc.). The computer may be coupled to external computers, such as via an optional network connection, a wireless transceiver, or both.

Various input devices may include a keyboard and/or a pointing device such as a mouse. Other input devices are possible such as a microphone, joystick, pen, touch screen, scanner, digital camera, video camera, and the like. Further input devices can include sequencing machine(s) (e.g., massively parallel sequencer), fluoroscopes, and other laboratory equipment, etc. Suitable data storage devices may include any type of computer-readable media that can store data accessible by the computer, such as magnetic hard and floppy disk drives, optical disk drives, magnetic cassettes, tape drives, flash memory cards, digital video disks (DVDs), Bernoulli cartridges, RAMs, ROMs, smart cards, etc. Indeed, any medium for storing or transmitting computer-readable instructions and data may be employed, including a connection port to or node on a network such as a local area network (LAN), wide area network (WAN) or the Internet.

Aspects of the disclosure may be practiced in a variety of other computing environments. For example, a distributed computing environment with a network interface includes can include one or more user computers in a system where they may include a browser program module that permits the computer to access and exchange data with the Internet, including web sites within the World Wide Web portion of the Internet. User computers may include other program modules such as an operating system, one or more application programs (e.g., word processing or spread sheet applications), and the like. The computers may be general-purpose devices that can be programmed to run various types of applications, or they may be single-purpose devices optimized or limited to a particular function or class of functions. More importantly, while shown with network browsers, any application program for providing a graphical user interface to users may be employed, as described in detail below; the use of a web browser and web interface are only used as a familiar example here.

At least one server computer, coupled to the Internet or World Wide Web (“Web”), can perform much or all of the functions for receiving, routing and storing of electronic messages, such as web pages, data streams, audio signals, and electronic images that are described herein. While the Internet is shown, a private network, such as an intranet may indeed be preferred in some applications. The network may have a client-server architecture, in which a computer is dedicated to serving other client computers, or it may have other architectures such as a peer-to-peer, in which one or more computers serve simultaneously as servers and clients. A database or databases, coupled to the server computer(s), can store much of the web pages and content exchanged between the user computers. The server computer(s), including the database(s), may employ security measures to inhibit malicious attacks on the system, and to preserve integrity of the messages and data stored therein (e.g., firewall systems, secure socket layers (SSL), password protection schemes, encryption, and the like).

A suitable server computer may include a server engine, a web page management component, a content management component and a database management component, among other features. The server engine performs basic processing and operating system level tasks. The web page management component handles creation and display or routing of web pages. Users may access the server computer by means of a URL associated therewith. The content management component handles most of the functions in the embodiments described herein. The database management component includes storage and retrieval tasks with respect to the database, queries to the database, read and write functions to the database and storage of data such as video, graphics and audio signals.

Many of the functional units described herein have been labeled as modules, in order to more particularly emphasize their implementation independence. For example, modules may be implemented in software for execution by various types of processors. An identified module of executable code may, for instance, comprise one or more physical or logical blocks of computer instructions which may, for instance, be organized as an object, procedure, or function. The identified blocks of computer instructions need not be physically located together, but may comprise disparate instructions stored in different locations which, when joined logically together, comprise the module and achieve the stated purpose for the module.

A module may also be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

A module of executable code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set, or may be distributed over different locations including over different storage devices, and may exist, at least partially, merely as electronic signals on a system or network.

### System for Genotoxic Testing

The present invention further comprises a system (e.g. a networked computer system, a high throughput automated system, etc.) for processing a subject's sample, and transmitting the sequencing data via a wired or wireless network to a remote server to determine the sample's error-corrected sequence reads (e.g., duplex sequence reads, duplex consensus sequence, etc.), mutation spectrum, mutant frequency, triplet mutation signature, and if there is a similarity between the sample data and corresponding data associated with one or more known genotoxins.

As described in additional detail below, and with respect to the embodiment illustrated in FIG. 19, a genotoxin computerized system comprises: (1) a remote server; (2) a plurality of user electronic computing devices able to generate and/or transmit sequencing data; (3) a database with known genotoxin profiles and associated information (optional); and (4) a wired or wireless network for transmitting electronic communications between the electronic computing devices, database, and the remote server. The remote server further comprises: (a) a database storing user genotoxin record results, and records of genotoxin profiles (e.g. spectrum, frequencies, mechanism of actions, etc.); (b) one or more processors communicatively coupled to a memory; and one or more non-transitory computer-readable storage devices or medium comprising instructions for processor(s), wherein said processors are configured to execute said instructions to perform operations comprising one or more of the steps described in FIGS. 20-23.

In one embodiment, the present technology further comprises, a non-transitory computer-readable storage media comprising instructions that, when executed by one or more processors, performs a method for determining if a subject is exposed to and/or the identity or properties/characteristics of at least one genotoxin. In particular embodiments, the methods can include one or more of the steps described in FIGS. 20-23.

Additional aspects of the present technology are directed to computerized methods for determining if a subject is exposed to and/or the identity or properties/characteristics of at least one genotoxin. In particular embodiments, the methods can include one or more of the steps described in FIGS. 20-23.

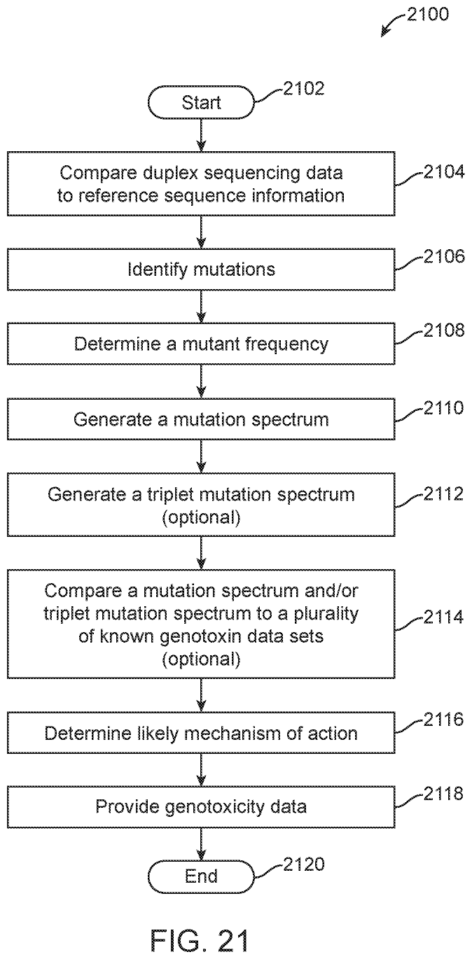

FIG. 19 is a block diagram of a computer system 1900 with a computer program product 1950 installed thereon and for use with the methods and/or kits disclosed herein to identify mutagenic events and/or nucleic acid damage events resulting from genotoxic exposure. Although FIG. 19 illustrates various computing system components, it is contemplated that other or different components known to those of ordinary skill in the art, such as those discussed above, can provide a suitable computing environment in which aspects of the disclosure can be implemented. FIG. 20 is a flow diagram illustrating a routine for providing Duplex Sequencing consensus sequence data in accordance with an embodiment of the present technology. FIGS. 21-23 are flow diagrams illustrating various routines for identifying mutagenic events and/or nucleic acid damage events resulting from genotoxic exposure of a sample. In accordance with aspects of the present technology, methods described with respect to FIGS. 21-23 can provide sample data including, for example, a sample's mutation spectrum, mutant frequency, triplet mutation spectrum, and information derived from comparison of sample data to data sets of known genotoxins.

As illustrated in FIG. 19, the computer system 1900 can comprise a plurality of user computing devices 1902, 1904; a wired or wireless network 1910 and a remote server (“DupSeg™” server) 1940 comprising processors to analyze mutagenic events and/or nucleic acid damage events resulting from genotoxic exposure of a sample. In embodiments, user computing devices 1902, 1904 can be used to generate and/or transmit sequencing data. In one embodiment, users of computing devices 1902, 1904 may be those performing other aspects of the present technology such as Duplex Sequencing method steps of subject samples for assessing genotoxicity. In one example, users of computing devices 1902, 1904 perform certain Duplex Sequencing method steps with a kit (1, 2) comprising reagents and/or adapters, in accordance with an embodiment of the present technology, to interrogate subject samples.

As illustrated, each user computing device 1902, 1904 includes at least one central processing unit 1906, a memory 1907 and a user and network interface 1908. In an embodiment, the user devices 1902, 1904 comprise a desktop, laptop, or a tablet computer.

Although two user computing devices 1902, 1904 are depicted, it is contemplated that any number of user computing devices may be included or connected to other components of the system 1900. Additionally, computing devices 1902, 1904 may also be representative of a plurality of devices and software used by User (1) and User (2) to amplify and sequence the samples. For example, a computing device may a sequencing machine (e.g., Illumina HiSeg™, Ion Torrent PGM, ABI SOLiD™ sequencer, PacBio RS, Helicos Heliscope™, etc.), a real-time PCR machine (e.g., ABI 7900, Fluidigm BioMark™, etc.), a microarray instrument, etc.

In addition to the above described components, the system 1900 may further comprise a database 1930 for storing genotoxin profiles and associated information. For example, the database 1930, which can be accessible by the server 1940, can comprise records or collections of mutation spectrum, triplet mutation spectrum/signatures, mechanism of action, etc. for a plurality of known genotoxins, and may also include additional information regarding mutation profiles/patterns of each stored genotoxin. In a particular example, the database 1930 can be a third-party database comprising genotoxin profiles 1932. For example, the Catalogue of Somatic Mutations in Cancer (COSMIC) website comprises a collection of “mutational spectrums” that have been found as clonal mutations in tumors that have arisen from exposure to carcinogens, e.g. lung cancers in smokers [8,9]. In another embodiment, the database can be a standalone database 1930 (private or not private) hosted separately from server 1940, or a database can be hosted on the server 1940, such as database 1970, that comprises empirically-derived genotoxin profiles 1972. In some embodiments, as the system 1900 is used to generate new test agent/factor profiles, the data generated from use of the system 1900 and associated methods (e.g., methods described herein and, for example, in FIGS. 20-23), can be uploaded to the database 1930 and/or 1970 so additional genotoxin profiles 1932, 1972 can be created for future comparison activities.

The server 1940 can be configured to receive, compute and analyze sequencing data (e.g., raw sequencing files) and related information from user computing devices 1902, 1904 via the network 1910. Sample-specific raw sequencing data can be computed locally using a computer program product/module (Sequence Module 1905) installed on devices 1902,1904, or accessible from the remote server 1940 via the network 1910, or using other sequencing software well known in the art. The raw sequence data can then be transmitted via the network 1910 to the remote server 1940 and user results 1974 can be stored in database 1970. The server 1940 also comprises program product/module “DS Module” 1912 configured to receive the raw sequencing data from the database 1970 and configured to computationally generate error corrected double-stranded sequence reads using, for example, Duplex Sequencing techniques disclosed herein. While DS Module 1912 is shown on server 1940, one of ordinary skill in the art would recognize that DS Module 1912 can alternatively, be hosted at operated at devices 1902, 1904 or on another remote server (not shown).

The remote server 1940 can comprise at least one central processing unit (CPU) 1960, a user and a network interface 1962 (or server-dedicated computing device with interface connected to the server), a database 1970, such as described above, with a plurality of computer files/records to store mutation profiles of known and novel genotoxins 1972, and files/records to store results (e.g., raw sequencing data, Duplex Sequencing data, genotoxicity analysis, etc.) for tested samples 1974. Server 1940 further comprises a computer memory 1911 having stored thereon the Genotoxin Computer Program Product (Genotoxin Module) 1950, in accordance with aspects of the present technology.

Computer program product/module 1950 is embodied in a non-transitory computer readable medium that, when executed on a computer (e.g. server 1940), performs steps of the methods disclosed herein for detecting and identifying genotoxins. Another aspect of the present disclosure comprises the computer program product/module 1950 comprising a non-transitory computer-usable medium having computer-readable program codes or instructions embodied thereon for enabling a processor to carry out genotoxicity analysis (e.g. compute mutant frequency, mutation spectrum, triplet mutation spectrum, genotoxin comparison reports, threshold level reports, etc.). These computer program instructions may be loaded onto a computer or other programmable apparatus to produce a machine, such that the instructions which execute on the computer or other programmable apparatus create means for implementing the functions or steps described herein. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Furthermore, computer program product/module 1950 may be implemented in any suitable language and/or browsers. For example, it may be implemented with Python, C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

The system invokes a number of routines. While some of the routines are described herein, one skilled in the art is capable of identifying other routines the system could perform. Moreover, the routines described herein can be altered in various ways. As examples, the order of illustrated logic may be rearranged, substeps may be performed in parallel, illustrated logic may be omitted, other logic may be included, etc.

FIGS. 20-23 are flow diagrams illustrating routines 2000, 2100, 2200, 2300 for detecting and identifying mutagenic events and/or nucleic acid damage events resulting from genotoxic exposure of a sample. FIG. 20 is a flow diagram illustrating routine 2000 for providing Duplex Sequencing Data for double-stranded nucleic acid molecules in a sample (e.g., a sample from a genotoxicity assay). The routine 2000 can be invoked by a computing device, such as a client computer or a server computer coupled to a computer network. In one embodiment the computing device includes sequence data generator and/or a sequence module. As an example, the computing device may invoke the routine 2000 after an operator engages a user interface in communication with the computing device.

The routine 2000 begins at block 2002 and the sequence module receives raw sequence data from a user computing device (block 2004) and creates a sample-specific data set comprising a plurality of raw sequence reads derived from a plurality of nucleic acid molecules in the sample (block 2006). In some embodiments, the server can store the sample-specific data set in a database for later processing. Next, the DS module receives a request for generating Duplex Consensus Sequencing data from the raw sequence data in the sample-specific data set (block 2008). The DS module groups sequence reads from families representing an original double-stranded nucleic acid molecule (e.g., based on SMI sequences) and compares representative sequences from individual strands to each other (block 2010). In one embodiment, the representative sequences can be one or more than one sequence read from each original nucleic acid molecule. In another embodiment, the representative sequences can be single-strand consensus sequences (SSCSs) generated from alignment and error-correction within representative strands. In such embodiments, a SSCS from a first strand can be compared to a SSCS from a second strand.

At block 2012, the DS module identifies nucleotide positions of complementarity between the compared representative strands. For example, the DS module identifies nucleotide positions along the compared (e.g., aligned) sequence reads where the nucleotide base calls are in agreement. Additionally, the DS module identifies positions of non-complementarity between the compared representative strands (block 2014). Likewise, the DS module can identify nucleotide positions along the compared (e.g., aligned) sequence reads where the nucleotide base calls are in disagreement.

Next, the DS module can provide Duplex Sequencing Data for double-stranded nucleic acid molecules in a sample (block 2016). Such data can be in the form of duplex consensus sequences for each of the processed sequence reads. Duplex consensus sequences can include, in one embodiment, only nucleotide positions where the representative sequences form each strand of an original nucleic acid molecule are in agreement. Accordingly, in one embodiment, positions of disagreement can be eliminated or otherwise discounted such that the duplex consensus sequence is a high accuracy sequence read that has been error-corrected. In another embodiment, Duplex Sequencing Data can include reporting information on nucleotide positions of disagreement in order that such positions can be further analyzed (e.g., in instances where DNA damage can be assessed.). The routine 2000 may then continue at block 2018 where it ends.

FIG. 21 is a flow diagram illustrating a routine 2100 for detecting and identifying mutagenic events resulting from genotoxic exposure of a sample. The routine can be invoked by the computing device of FIG. 20. The routine 2100 begins at block 2102 and the genotoxin module compares the Duplex Sequencing Data from FIG. 20 (e.g., following block 2016) to reference sequence information (block 2104) and identifies mutations (e.g., where the subject sequence varies from the reference sequence) (block 2106). Next, the genotoxin module determines a mutant frequency (block 2108) and generates a mutation spectrum (block 2110) for the sample. As such, a mutation pattern analysis can be provided with information regarding the type, location and frequency of mutation events in the nucleic acid molecules analyzed from the sample. Optionally, the genotoxin module can generate a triplet mutation spectrum (block 2112) providing trinucleotide context and pattern information for analyzing the genotoxic result of exposure.

The genotoxin module can also optionally compare a mutation spectrum and/or triplet mutation spectrum (if determined) to a plurality of known genotoxin data sets, such as those stored in genotoxin profile records in a database (block 2114) to determine, for example, if the sample was exposed to a known genotoxin, or in another example, to determine if a test agent/factor has a similar genotoxic profile as a previously known genotoxin. Optionally, the genotoxin module can determine a likely mechanism of action of a genotoxin based, in part, on the comparison information (block 2116). Next, the genotoxin module can provide genotoxicity data (block 2118) that can be stored in the sample-specific data set in the database. In some embodiments, not shown, the genotoxicity data can be used to generate a genotoxin profile to be stored in the database for future comparison activities. The routine 2100 may then continue at Mock 2120, where it ends.

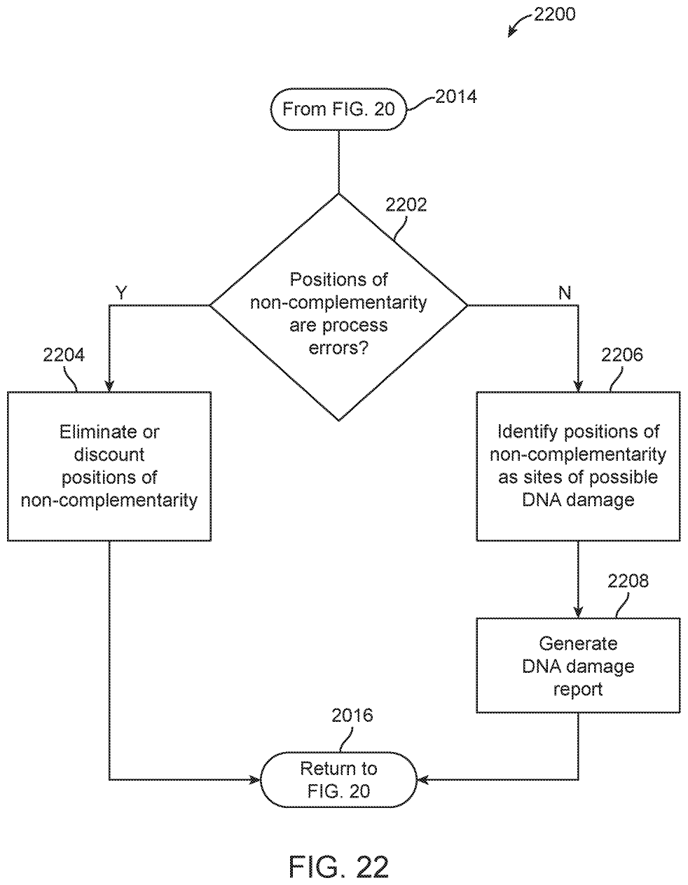

FIG. 22 is a flow diagram illustrating a routine 2200 for detecting and identifying DNA damage events resulting from genotoxic exposure of a sample. The routine can be invoked by the computing device of FIG. 20. The routine 2200 begins at block 2014 of FIG. 20 and at decision block 2202, the routine 2200 determines whether nucleotide positions of non-complementarity are process errors. In various embodiments, the parameters for determining whether a position of disagreement between the sequence reads of both strands of an original DNA molecule can be specified by an operator, by known characteristics of DNA damage, by known characteristics of process errors, by a minimum number of sequence reads the mismatch is represented by, and so forth.

If the nucleotide position is determined to be a process error (as opposed to a site of in vivo DNA damage prior to DNA extraction), the DS module can eliminate or discount such nucleotide positions of non-complementarity (block 2204). The routine 2200 can continue to block 2016 of FIG. 20.

Referring back to decision block 2202, and if the nucleotide position is determined to not be a process error, the genotoxin module can identify such positions of non-complementarity as sites of possible in vivo DNA damage (block 2206), such as resulting from exposure to a genotoxin. Following identification, the genotoxin module can generate a DNA damage report to be associated with the sample-specific data set in the database (block 2208). In some embodiments, the DNA damage report can be used to infer mechanism of action of a potential genotoxin (not shown). The routine 2200 can continue to block 2016 of FIG. 20.

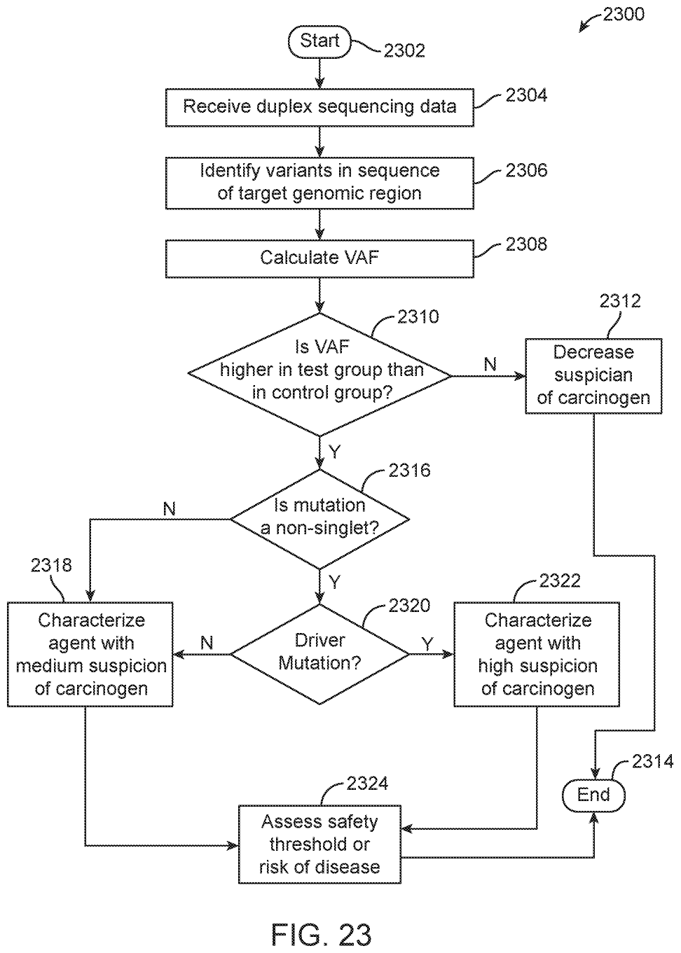

FIG. 23 is a flow diagram illustrating a routine 2300 for detecting and identifying a carcinogen or carcinogen exposure in a subject. The routine 2300 can be invoked by the computing device of FIG. 20. The routine 2300 begins at block 2302 and the genotoxin module receives Duplex Sequencing Data from FIG. 20 (e.g., following block 2016) and, optionally, genotoxicity data from FIG. 21 (e.g., following block 2116) and confirms that the sample was exposed to a genotoxin (block 2304). Next, the genotoxin module identifies variants in the sequence of a target genomic region (e.g., gene) (block 2306). For example, the genotoxin module can analyze Duplex Sequencing Data and genotoxicity data at specific genetic loci (e.g., cancer driver genes, oncogenes, etc.). Then, the genotoxin module calculates a variant allele frequency (VAF) (block 2308).

At decision block 2310, the routine 2300 determines whether the VAF is higher in a test group than in a control group. If the VAF of the test group is not higher than a control group, the genotoxin module labels the agent for decreased suspicion of being a carcinogen (block 2312). The routine 2300 may then continue at block 2314, where it ends. If the VAF is higher in the test group than in the control group, the routine 2300 continues at decision block 2316, where the routine 2300 determines if a mutation is a non-singlet.

If the mutation is a singlet, then the genotoxin module characterizes the agent with a medium level of suspicion of being a carcinogen (block 2318). If the mutation is determined to be a non-singlet (i.e., a multiplet), the routine continues at decision block 2320, wherein the routine 2300 determines if a variant is detected at target gene and if the variant is consistent with a driver mutation (e.g., a mutation known to drive cancer growth/transformation).

If the mutation is not a driver mutation, the genotoxin module characterizes the agent with a medium level of suspicion of being a carcinogen (block 2318). If the variant(s) are consistent with a driver mutation, the genotoxin module characterizes the agent with a high level of suspicion of being a carcinogen (block 2322).

For agents that have been characterized with either a medium level of suspicion (at block 2318) or a high level of suspicion (at block 2318), the genotoxin module can assess a safety threshold for the carcinogen and/or determine a risk associated with developing a genotoxin-associated disease or disorder following the exposure in the subject (block 2324). The routine 2300 may then continue at block 2314, where it ends.

Other steps and routines are also contemplated by the present technology. For example, the system (e.g., the genotoxin module or other module) can be configured to analyze the genotoxin data to determine if a subject was exposed to a genotoxin, if a test agent/factor is genotoxic, determine under what characteristics a genotoxin is mutagenic or carcinogenic and the like. Other steps may include determining if a subject should be prophylactically or therapeutically treated based on the genotoxin data derived from a particular subject's biological sample. For example, once the genotoxin(s) is identified using the system, the server can then determine if the subject has been exposed to more than a safe threshold level of genotoxin. If so, then a prophylactic or inhibitor disease treatments may be initiated.

### Additional Examples

1. A method for detecting and quantifying genomic mutations developed in vivo in a subject following the subject's exposure to a mutagen, comprising:
providing a sample from the subject, wherein the sample comprises double-stranded DNA molecules;
generating an error-corrected sequence read for each of a plurality of the double-stranded DNA molecules in the sample, comprising:
   generating a set of copies of an original first strand of the adapter-DNA molecule and a set of copies of an original second strand of the adapter-DNA molecule;
   sequencing the set of copies of the original first and second strands to provide a first strand sequence and a second strand sequence; and
   comparing the first strand sequence and the second strand sequence to identify one or more correspondences between the first and second strand sequences; and
analyzing the one or more correspondences to determine a mutation spectrum for the double-stranded DNA molecules in the sample.

2. The method of example 1, further comprising calculating a mutant frequency for the target double-stranded DNA molecules by calculating the number of unique mutations per duplex base-pair sequenced.

3. The method of example 1, wherein the target double-stranded DNA molecules were extracted from liver, spleen, blood, lung or bone marrow of the subject.

4. The method of example 1, wherein the subject was exposed to the mutagen 30 days or less prior to the target double-stranded DNA molecules being removed from the subject.

5. The method of example 1, wherein the mutation spectrum is generated by unsupervised hierarchical mutation spectrum clustering.

6. The method of example 1, wherein the mutation spectrum is a triplet mutation spectrum.

7. The method of example 1, wherein generating an error-corrected sequence read for each of a plurality of the double-stranded DNA molecules includes generating error-corrected sequence reads of one or more targeted genomic regions.

8. The method of example 7, wherein the one or more targeted genomic regions is a mutation-prone site in the genome.

9. The method of example 7, wherein the one or more targeted genomic regions is a known cancer driver gene.

10. The method of example 1, wherein the subject is a transgenic animal, and wherein at least some of the target double-stranded DNA molecules include one or more portions of a transgene.

11. The method of example 1, wherein the subject is a non-transgenic animal, and wherein the target double-stranded DNA molecules comprise endogenous genomic regions.

12. The method of example 1, wherein the subject is a human, and wherein the target double-stranded DNA molecules are extracted from a blood draw taken from the human.

13. A method for generating a mutagenic signature of a test agent, comprising:
duplex sequencing DNA fragments extracted from a test subject exposed to the test agent; and
generating a mutagenic signature of the test agent, comprising:
   calculating a mutant frequency for a plurality of the DNA fragments by calculating the number of unique mutations per duplex base-pair sequenced; and
   determining a mutation pattern for the plurality of the DNA fragments, wherein the mutation pattern includes mutation type, mutation trinucleotide context, and genomic distribution of mutations.

14. The method of example 13, further comprising comparing the mutation signature of the test agent with mutation signatures of one or more known genotoxins.

15. The method of example 13, wherein the mutation signature of the test agent varies based on one or more of a tissue type, a level of exposure to the test agent, a genomic region, and a subject type.

16. The method of example 15, wherein the subject type is human cells grown in culture.

17. The method of example 13, wherein the test animal was exposed to the test compound 30 days or less prior to the animal being sacrificed.

18. The method of example 13, wherein the mutagenic signature is generated by computational pattern matching.

19. The method of example 13, wherein the mutation signature is a triplet mutation signature.

20. The method of example 13, wherein duplex sequencing DNA fragments includes duplex sequencing one or more targeted genomic regions.

21. The method of example 20, wherein the one or more targeted genomic regions is a mutation-prone site in the genome.

22. The method of example 20, wherein the one or more targeted genomic regions is a known cancer driver gene.

23. The method of example 13, wherein the test animal is a transgenic animal, and wherein at least some of the DNA fragments include one or more portions of a transgene.

24. The method of example 13, wherein the test animal is a non-transgenic animal, and wherein the DNA fragments comprise endogenous genomic regions.

25. A method for assessing a genotoxic potential of a test agent, comprising:
(a) preparing a sequencing library from a sample comprising a plurality of double-stranded DNA fragments from a biological source exposed to the test agent, wherein preparing the sequence library comprises ligating asymmetric adapter molecules to the plurality of double-stranded DNA fragments to generate a plurality of adapter-DNA molecules;
(b) sequencing first and second strands of the adapter-DNA molecules to provide a first strand sequence read and a second strand sequence read for each adapter-DNA molecule;
(c) for each adapter-DNA molecule, comparing the first strand sequence read and the second strand sequence read to identify one or more correspondences between the first and second strand sequences reads; and
(d) determining a mutation signature of the test agent by analyzing the one or more correspondences between the first and second strand sequences reads for each of the adapter-DNA molecules to determine at least one of a mutation pattern, a mutation type, a mutant frequency, a mutation type distribution, and a genomic distribution of mutations in the sample; and
(e) comparing the mutation signature of the test agent to a plurality of mutation spectra derived from known genotoxins to determine if the mutation signature is sufficiently similar to a mutation spectrum from a known genotoxin; or
(f) assessing if at least one of the mutant frequency, the mutations type, or the mutation type distribution is above a safe threshold level; or
(g) determining if the mutant frequency exceeds a safe threshold mutant frequency.

26. The method of example 25 wherein a mutation signature of the test agent comprises a mutant frequency above a safe threshold frequency.

27. The method of example 25, wherein the mutation signature of the test agent comprises a mutation pattern sufficiently similar to known cancer-associated mutation pattern.

28. The method of example 25, wherein the biological source is at least one of cells grown in culture, an animal, a human, a human cell line, a transgenic animal, a non-transgenic animal, a human tissue sample, or a human blood sample.

29. The method of example 25, wherein the biological source was exposed to the test agent 30 days or less prior to extracting the sample comprising a plurality of double-stranded DNA fragments.

30. The method of example 25, wherein the mutation signature is a triplet mutation signature.

31. The method of example 25, wherein prior to comparing the first strand sequence read and the second strand sequence read, the method comprises associating the first strand sequence read with the second strand sequence read using one or more of an adapter sequence, sequence read length, and original strand information.

32. The method of example 25, wherein prior to preparing the sequencing library, the method further comprises exposing the biological source to the test agent.

33. The method of example 32, wherein prior to exposing the biological source to the test agent, the biological source is or comprises a cancer tissue.

34. The method of example 32, wherein prior to exposing the biological source to the test agent, the biological source is or comprises a healthy tissue.

35. The method of example 25, wherein the sample is or comprises a blood sample.

36. The method of example 25, wherein the sample is or comprises a cancer cell line.

37. The method of example 25, wherein the biological source comprises cancerous cells, and wherein the substance is tested for selective genotoxicity to at least a portion of the cancerous cells.

38. The method of example 37, wherein the substance is a therapeutic compound.

39. The method of example 38, wherein for the portion of the cancerous cells shown to be sensitive to the selective genotoxicity of the therapeutic compound, the method further comprises determining one or more of a mutant frequency and a mutation spectrum for the portion of the cancerous cells prior to exposure to the therapeutic compound.

40. The method of example 25, wherein the test agent comprises a food, a drug, a vaccine, a cosmetic substance, an industrial additive, an industrial by-product, petroleum distillate, heavy metal, household cleaner, airborne particulate, byproduct of manufacturing, contaminant, plasticizer, detergent, a radiation-emitting product, a tobacco product, a chemical material, or a biological material.

41. A method for determining a subject's exposure to a genotoxic agent, comprising:
comparing a subjects' DNA mutation spectrum with mutation spectra of known mutagenic compounds; and
identifying the mutation spectra of known mutagenic compounds most similar to the subject's DNA mutation spectrum.

42. The method of example 41, wherein the subject's DNA mutation spectrum is assessed by Duplex Sequencing.

43. The method of example 41, wherein the subject's DNA mutation spectrum is generated from DNA extracted from the patient's blood.

44. The method of example 41, wherein the subject's DNA mutation spectrum is a triplet mutation spectrum.

45. The method of example 41, further comprising sequencing the subject's DNA to generate the subject's DNA mutation spectrum.

46. The method of example 45, wherein sequencing the subject's DNA includes sequencing one or more known cancer driver genes.

47. A kit able to be used in error corrected duplex sequencing of double stranded polynucleotides to identify genotoxins, the kit comprising:
at least one set of polymerase chain reaction (PCR) primers and at least one set of adaptor molecules, wherein the primers and adaptor molecules are able to be used in error corrected duplex sequencing experiments; and
instructions on methods of use of the kit in conducting error corrected duplex sequencing of DNA extracted from a subject's sample to identify if the subject has been exposed to at least one genotoxin.

48. The kit of example 47, wherein the reagent comprises a DNA repair enzyme.

49. The kit of example 47, wherein each of the adapter molecules in the set of adaptor molecules comprises at least one single molecule identifier (SMI) sequence and at least one strand defining element.

50. The kit of example 47, further comprises a computer program product embodied in a non-transitory computer readable medium that, when executed on a computer, performs steps of determining an error-corrected duplex sequencing read for one or more double-stranded DNA molecules in a sample, and determining the mutant frequency, mutation spectrum, and/or triplet spectrum of at least one genotoxin using the error-corrected duplex sequencing read.

51. The kit of example 50, wherein the computer program product further determines the mechanism of action of the genotoxin in mutating a subject's DNA; and therapeutic or prophylactic treatments suitable for administering to the subject based upon the genotoxin mechanism of action.

52. A method for diagnosing and treating a subject exposed to a genotoxin, comprising:
a) determining whether a subject was exposed to a genotoxin by:
   i) obtaining a biological sample from the subject;
   ii) providing duplex error corrected sequencing reads for a plurality of double stranded DNA sequences extracted from the sample;
   iii) determining the mutant frequency, mutation spectrum, and/or triplet mutation spectrum of the DNA sequences;
   iv) determining if the mutant frequency, mutation spectrum and/or triplet mutation spectrum is indicative of the subject having been exposed to a genotoxin;
b) if the subject has been exposed to the genotoxin, then providing a prophylactic and/or a therapeutic treatment to prevent or inhibit the onset of a disease or disorder associated with the genotoxin.

53. A method for identifying a threshold level of safe exposure to a genotoxin, and providing treatment, comprising:
a) determining a genotoxin's threshold level of safe exposure;
b) determining whether a subject was exposed to the genotoxin at a level greater than the threshold level of safe exposure by:
   i) obtaining a biological sample from the subject;
   ii) providing duplex error corrected sequencing reads for a plurality of double stranded DNA sequences extracted from the biological sample;
   iii) determining the mutant frequency, mutation spectrum, and/or triplet mutation spectrum of the DNA sequences;
   iv) determining if the mutant frequency, mutation spectrum and/or triplet mutation spectrum are indicative of the subject having been exposed to a specific genotoxin;
v) computing the level of exposure of the subject to the genotoxin based on the mutant frequency, mutation spectrum and/or triplet mutation spectrum; and
c) if the subject has been exposed to more than the genotoxin's threshold level of safe exposure, then providing a prophylactic and/or a therapeutic treatment to prevent or inhibit the onset of a disease or disorder associated with the genotoxin.

54. A system for detecting and identifying mutagenic events and/or nucleic acid damage events resulting from genotoxic exposure of a sample, comprising:
a computer network for transmitting information relating to sequencing data and genotoxicity data, wherein the information includes one or more of raw sequencing data, duplex sequencing data, sample information, and genotoxin information;
a client computer associated with one or more user computing devices and in communication with the computer network;
a database connected to the computer network for storing a plurality of genotoxin profiles and user results records;
a duplex sequencing module in communication with the computer network and configured to receive raw sequencing data and requests from the client computer for generating duplex sequencing data, group sequence reads from families representing an original double-stranded nucleic acid molecule and compare representative sequences from individual strands to each other to generate duplex sequencing data; and
a genotoxin module in communication with the computer network and configured to compare duplex sequencing data to reference sequence information to identify mutations and generate genotoxin data comprising at least one of a mutant frequency, a mutation spectrum, and a triplet mutation spectrum.

55. The system of example 54, wherein the genotoxin profiles comprise genotoxin mutation spectrum from a plurality of known genotoxins.

56. A non-transitory computer-readable storage medium comprising instructions that, when executed by one or more processors, performs a method of any one of examples 1-53 for determining if a subject is exposed to at least one genotoxin and/or determining an identity of at least one genotoxin.

57. The non-transitory computer-readable storage medium of example 56, further comprising computing the mutation spectrum, mutant frequency, and/or triplet mutation spectrum of a detected agent, from which the identity of the at least one genotoxin is determined.

58. A computer system for performing a method of any one of examples 1-53 for determining if a subject is exposed to and/or an identity of at least one genotoxin, the system comprising: at least one computer with a processor, memory, database, and a non-transitory computer readable storage medium comprising instructions for the processor(s), wherein said processor(s) are configured to execute said instructions to perform operations comprising the methods of any one of examples 1-53.

59. The system of example 58, further comprising a networked computer system comprising:
a. a wired or wireless network;
b. a plurality of user electronic computing devices able to receive data derived from use of a kit comprising reagents to extract, amplify, and produce a polynucleotide sequence of a subject's sample, and to transmit the polynucleotide sequence via a network to a remote server; and
c. a remote server comprising the processor, memory, database, and the non-transitory computer readable storage medium comprising instructions for the processor(s), wherein said processor(s) are configured to execute said instructions to perform operations comprising the methods of any one of examples 1-53; and
d. wherein said remote server is able to detect and identify mutagenic events and/or nucleic acid damage events resulting from genotoxic exposure of a sample.

60. The system of example 59, wherein the database and/or a third-party database accessible via the network, further comprises a plurality of records comprising one or more of a genotoxin profile of known genotoxins, a genotoxin profile of at least one subject's sample, and wherein the genotoxin profile comprises a mutation or a site of DNA damage.

61. A non-transitory computer-readable medium whose contents cause at least one computer to perform a method for providing duplex sequencing data for double-stranded nucleic acid molecules in a sample from a genotoxicity screening assay, the method comprising:
receiving raw sequence data from a user computing device; and
creating a sample-specific data set comprising a plurality of raw sequence reads derived from a plurality of nucleic acid molecules in the sample;
grouping sequence reads from families representing an original double-stranded nucleic acid molecule, wherein the grouping is based on a shared single molecule identifier sequence;
comparing a first strand sequence read and a second strand sequence read from an original double-stranded nucleic acid molecule to identify one or more correspondences between the first and second strand sequences reads; and
providing duplex sequencing data for the double-stranded nucleic acid molecules in the sample.

62. The computer-readable medium of example 58, further comprising identifying nucleotide positions of non-complementarity between the compared first and second sequence reads, wherein the method further comprises:
in positions of non-complementarity, identifying and eliminating or discounting process errors; and
in positions of non-complementarity that are not identified as process errors, identifying remaining positions of non-complementarity as sites of possible in vivo DNA damage resulting from exposure to a genotoxin.

63. A non-transitory computer-readable medium whose contents cause at least one computer to perform a method for detecting and identifying mutagenic events resulting from genotoxic exposure of a sample, the method comprising:
comparing duplex sequence data to reference sequence information;
identify mutations in the duplex sequence data, wherein a mutation is identified as a region of non-agreement with the reference information;
determining a mutant frequency in the duplex sequence data;
generating a mutation spectrum from the duplex sequence data;
generating a triplet mutation spectrum from the duplex sequence data; and
compare the mutation spectrum and/or the triplet mutation spectrum to a plurality of known genotoxin data sets.

64. A non-transitory computer-readable medium whose contents cause at least one computer to perform a method for detecting and identifying a carcinogen or carcinogen exposure in a subject, the method comprising:
identifying sequence variants in a target genomic region using duplex sequencing data generated from a sample from the subject;
calculating a variant allele frequency (VAF) of a test sample and a control sample;
determining if a VAF is higher in a test group than in a control group;
in samples having a higher VAF, determining if a sequence variant is a non-singlet;
in samples having a higher VAF, determining if the sequence variant is a driver mutation; and
characterizing samples having a non-singlet and/or a driver mutation as being suspicious for being a carcinogen.

65. The non-transitory computer-readable medium of example 68, further comprising assessing a safety threshold for the carcinogen and/or determining a risk associated with developing a genotoxin-associated disease or disorder following the exposure in the subject.

### REFERENCES

The references listed below, as well as patents, and published patent applications cited in the specification above, are hereby incorporated by reference in their entirety, as if fully set forth herein.
[1] Schmitt M W, Kennedy S R, Salk J J, Fox E J, Hiatt J B, and Loeb L A. Detection of ultra-rare mutations by next-generation sequencing. Proc Natl Acad Sci USA. 2012; 109(36): 14508-14513.
[2] Kennedy S R, Salk J J, Schmitt M W, Loeb L A. Ultra-Sensitive Sequencing Reveals an Age-Related Increase in Somatic Mitochondrial Mutations that are inconsistent with oxidative damage. PLOS Genetics. 2013; 9(9): 1-10.
[3] Kennedy S R, Schmitt M W, Fox E J, Kohrn B F, Salk J J, Ahn E H, et al. Detecting ultralow-frequency mutations by Duplex Sequencing. Nat Protoc. 2014; 9(11): 2586-2606.
[4] Schmitt M W, Fox E J, Prindle M J, Reid-Bayliss K S, True L D, et al. Sequencing small genomic targets with high efficiency and extreme accuracy. Nature Methods. 2015; 12(5): 423-5.
[5] Chan C Y, Huang P H, Guo F, Ding X, Kapur V, Mai J D, et al. Accelerating drug discovery via organs-on-chips. Lab Chip. 2013; 12(24): 4697-4710.
[6] Schmitt M W, Loeb L A, and Salk J J. The influence of subclonal resistance mutations on targeted cancer therapy. Nat Rev Clin Oncol. 2016; 13(6): 335-347.
[7] Salk J J, Schmitt M W, Loeb L A. Enhancing the accuracy of next-generation sequencing for detecting rare and subclonal mutations. Nature Reviews Genetics. 2018. 19:269-283.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments. All references cited herein are incorporated by reference as if fully set forth herein.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

The product names used in this disclosure are for identification purposes only. All trademarks are the property of their respective owners.

1. A method for detecting and quantifying genomic mutations developed in vivo in a subject following the subject's exposure to a mutagen, comprising:
providing a sample from the subject, wherein the sample comprises double-stranded DNA molecules;
generating an error-corrected sequence read for each of a plurality of the double-stranded DNA molecules in the sample, comprising:
generating a set of copies of an original first strand of the adapter-DNA molecule and a set of copies of an original second strand of the adapter-DNA molecule;
sequencing the set of copies of the original first and second strands to provide a first strand sequence and a second strand sequence; and
comparing the first strand sequence and the second strand sequence to identify one or more correspondences between the first and second strand sequences; and
analyzing the one or more correspondences to determine a mutation spectrum for the double-stranded DNA molecules in the sample.

2. The method of claim 1, further comprising calculating a mutant frequency for the target double-stranded DNA molecules by calculating the number of unique mutations per duplex base-pair sequenced.

3. The method of claim 1, wherein the target double-stranded DNA molecules were extracted from liver, spleen, blood, lung or bone marrow of the subject.

4. The method of claim 1, wherein the subject was exposed to the mutagen 30 days or less prior to the target double-stranded DNA molecules being removed from the subject.

5. The method of claim 1, wherein the mutation spectrum is generated by unsupervised hierarchical mutation spectrum clustering.

6. The method of claim 1, wherein the mutation spectrum is a triplet mutation spectrum.

7. The method of claim 1, wherein generating an error-corrected sequence read for each of a plurality of the double-stranded DNA molecules includes generating error-corrected sequence reads of one or more targeted genomic regions.

8-11. (canceled)

12. The method of claim 1, wherein the subject is a human, and wherein the target double-stranded DNA molecules are extracted from a blood draw taken from the human.

13. A method for generating a mutagenic signature of a test agent, comprising:
duplex sequencing DNA fragments extracted from a test subject exposed to the test agent; and
generating a mutagenic signature of the test agent, comprising:
calculating a mutant frequency for a plurality of the DNA fragments by calculating the number of unique mutations per duplex base-pair sequenced; and
determining a mutation pattern for the plurality of the DNA fragments, wherein the mutation pattern includes mutation type, mutation trinucleotide context, and genomic distribution of mutations.

14-24. (canceled)

25. A method for assessing a genotoxic potential of a test agent, comprising:
(a) preparing a sequencing library from a sample comprising a plurality of double-stranded DNA fragments from a biological source exposed to the test agent, wherein preparing the sequence library comprises ligating asymmetric adapter molecules to the plurality of double-stranded DNA fragments to generate a plurality of adapter-DNA molecules;
(b) sequencing first and second strands of the adapter-DNA molecules to provide a first strand sequence read and a second strand sequence read for each adapter-DNA molecule;
(c) for each adapter-DNA molecule, comparing the first strand sequence read and the second strand sequence read to identify one or more correspondences between the first and second strand sequence reads; and
(d) determining a mutation signature of the test agent by analyzing the one or more correspondences between the first and second strand sequence reads for each of the adapter-DNA molecules to determine at least one of a mutation pattern, a mutation type, a mutant frequency, a mutation type distribution, and a genomic distribution of mutations in the sample; and
(e) comparing the mutation signature of the test agent to a plurality of mutation spectra derived from known genotoxins to determine if the mutation signature is sufficiently similar to a mutation spectrum from a known genotoxin; or
(f) assessing if at least one of the mutant frequency, the mutations type, or the mutation type distribution is above a safe threshold level; or
(g) determining if the mutant frequency exceeds a safe threshold mutant frequency.

26-40. (canceled)

41. A method for determining a subject's exposure to a genotoxic agent, comprising:
comparing a subjects' DNA mutation spectrum with mutation spectra of known mutagenic compounds; and
identifying the mutation spectra of known mutagenic compounds most similar to the subject's DNA mutation spectrum.

42-46. (canceled)

47. A kit for identifying exposure to a genotoxin in a subject, the kit comprising:
at least one set of polymerase chain reaction (PCR) primers and at least one set of adaptor molecules, wherein the primers and the adaptor molecules are able to be used in an error-corrected duplex sequencing assay; and
instructions on methods for using the kit in conducting error-corrected duplex sequencing of DNA extracted from a sample from the subject to identify if the subject has been exposed to at least one genotoxin.

48. The kit of claim 47, wherein the kit further comprises a DNA repair enzyme.

49. The kit of claim 47, wherein each of the adapter molecules in the set of adaptor molecules comprises at least one single molecule identifier (SMI) sequence and at least one strand defining element.

50. The kit of claim 47, further comprising a computer program product embodied in a non-transitory computer readable medium that, when executed on a computer, performs steps of determining an error-corrected duplex sequencing read for one or more double-stranded DNA molecules in a sample, and determining the mutant frequency, mutation spectrum, and/or triplet spectrum of at least one genotoxin using the error-corrected duplex sequencing read.

51. The kit of claim 50, wherein the computer program product further determines the mechanism of action of the genotoxin in mutating a subject's DNA; and therapeutic or prophylactic treatments suitable for administering to the subject based upon the genotoxin mechanism of action.

52. A method for diagnosing and treating a subject exposed to a genotoxin, comprising:
a) determining whether a subject was exposed to a genotoxin by:
i) obtaining a biological sample from the subject;
ii) providing duplex error-corrected sequencing reads for a plurality of double stranded DNA sequences extracted from the sample;
iii) determining the mutant frequency, mutation spectrum, and/or triplet mutation spectrum of the DNA sequences;
iv) determining if the mutant frequency, mutation spectrum and/or triplet mutation spectrum is indicative of the subject having been exposed to a genotoxin;
b) if the subject has been exposed to the genotoxin, then providing a prophylactic and/or a therapeutic treatment to prevent or inhibit the onset of a disease or disorder associated with the genotoxin.

53. A method for identifying a threshold level of safe exposure to a genotoxin, and providing treatment, comprising:
a) determining a genotoxin's threshold level of safe exposure;
b) determining whether a subject was exposed to the genotoxin at a level greater than the threshold level of safe exposure by:
i) obtaining a biological sample from the subject;
ii) providing duplex error-corrected sequencing reads for a plurality of double stranded DNA sequences extracted from the biological sample;
iii) determining the mutant frequency, mutation spectrum, and/or triplet mutation spectrum of the DNA sequences;
iv) determining if the mutant frequency, mutation spectrum and/or triplet mutation spectrum are indicative of the subject having been exposed to a specific genotoxin;
v) computing the level of exposure of the subject to the genotoxin based on the mutant frequency, mutation spectrum and/or triplet mutation spectrum; and
c) if the subject has been exposed to more than the genotoxin's threshold level of safe exposure, then providing a prophylactic and/or a therapeutic treatment to prevent or inhibit the onset of a disease or disorder associated with the genotoxin.

54. A system for detecting and identifying mutagenic events and/or nucleic acid damage events resulting from genotoxic exposure of a sample, comprising:
a computer network for transmitting information relating to sequencing data and genotoxicity data, wherein the information includes one or more of raw sequencing data, duplex sequencing data, sample information, and genotoxin information;
a client computer associated with one or more user computing devices and in communication with the computer network;
a database connected to the computer network for storing a plurality of genotoxin profiles and user results records;
a duplex sequencing module in communication with the computer network and configured to receive raw sequencing data and requests from the client computer for generating duplex sequencing data, group sequence reads from families representing an original double-stranded nucleic acid molecule and compare representative sequences from individual strands to each other to generate duplex sequencing data; and
a genotoxin module in communication with the computer network and configured to compare duplex sequencing data to reference sequence information to identify mutations and generate genotoxin data comprising at least one of a mutant frequency, a mutation spectrum, and a triplet mutation spectrum.

55. (canceled)

56. A non-transitory computer-readable storage medium comprising instructions that, when executed by one or more processors, performs a method of claim 1 for determining if a subject is exposed to at least one genotoxin and/or determining an identity of at least one genotoxin.

57-65. (canceled)
